SEQUENCE LISTING (20 sequences)

SEQ ID NO: 1 - RNA, 69 nt, Homo sapiens
gggauacucaaaaugggggcgcuuuccuuuuugucuguacugggaagugcuucgauuuugggguguccc

SEQ ID NO: 2 - RNA, 22 nt, Homo sapiens
acucaaaaugggggcgcuuucc

SEQ ID NO: 3 - RNA, 23 nt, Homo sapiens
gaagugcuucgauuuuggggugu

SEQ ID NO: 4 - DNA, 54 bp, Homo sapiens
ctcgagatctggggatactcaaaatgggggcgctttcctttttgtctgtactgg

SEQ ID NO: 5 - DNA, 53 bp, Homo sapiens
ctcgaggatccgggacaccccaaaatcgaagcacttcccagtacagacaaaaa

SEQ ID NO: 6 - DNA, 24 bp, Homo sapiens
gctgcagttgcaactatgcacttg

SEQ ID NO: 7 - DNA, 24 bp, Mus musculus
atttcagttgcaactatgcacttg

SEQ ID NO: 8 - DNA, 24 bp, Rattus norvegicus
atttcagttgcaactatgcacttg

SEQ ID NO: 9 - DNA, 24 bp, Equus caballus
actgcagttgcaactatgcacttg

SEQ ID NO: 10 - DNA, 24 bp, Ovis aries
actgcagttgcaactatgcacttg

SEQ ID NO: 11 - DNA, 24 bp, Gallus gallus
attgcagttgcaactatgcacttg

SEQ ID NO: 12 - DNA, 23 bp, Homo sapiens
tgtggggttttagcttcgtgaag

SEQ ID NO: 13 - RNA, 23 nt, Homo sapiens
acaccccaaaaucgaagcacuuc

SEQ ID NO: 14 - DNA, 69 bp, Artificial Sequence
  Plasmid vector from the company
gaattcactagtaccggtaggcctgtcgacgatatcgggcccgcggccgctggatcctctagactcgag

SEQ ID NO: 15 - DNA, 24 bp, Homo sapiens
gcugcaguugcaacuaugcacuug

SEQ ID NO: 16 - DNA, 23 bp, Homo sapiens
ugugggguuuuagcuucgugaag

SEQ ID NO: 17 - DNA, 23 bp, Homo sapiens
aatgggaaaacaactataaggcc

SEQ ID NO: 18 - DNA, 23 bp, Homo sapiens
acaccuacucacuuaagcacuug

SEQ ID NO: 19 - RNA, 23 nt, Homo sapiens
ugugggguuuuagcuucgugaag

SEQ ID NO: 20 - DNA, 22 bp, Homo sapiens
tttgggactcacttcccggaaa

(19) United States
(12) Patent Application Publication
(10) Pub. No.: US 2021/0054344 A1
(43) Pub. Date: Feb. 25, 2021

(54) CPC EXOSOMES MIRNA373 COMBINATION THERAPIES

(71) Applicant: IPS Heart, Sugar Land, TX (US)

(72) Inventor: Muhammad Ashraf, Cincinnati, OH (US)

(21) Appl. No.: 16/795,217

(22) Filed: Feb. 19, 2020

Related U.S. Application Data
(63) Continuation of application No. 15/881,693, filed on Jan. 26, 2018.
(60) Provisional application No. 62/807,647, filed on Feb. 19, 2019.

(51) Int. Cl. C12N 5/074; C12N 5/077; A61K 35/12; C12N 5/071; A61K 31/7105; A61K 45/06; A61K 31/713; A61K 35/545

(52) U.S. Cl. CPC C12N 5/0696; C12N 5/0657; C12N 5/0661; A61K 35/12; C12N 5/069; C12N 5/0658; C12N 2501/727; A61K 45/06; A61K 31/713; A61K 35/545; C12N 2506/45; C12N 2501/999; A61K 31/7105

(57) ABSTRACT

Steam cell and exosome compositions via combination therapy, related gene therapy and pluripotent stem cell derived muscle regeneration as having therapeutic utility to treat a variety of diseases and disorders, e.g., cardiovascular disease, Duchenne muscular dystrophy, and fibrotic disease.

### PRIOR RELATED APPLICATIONS

This application is a CIP of 62/807,647, INDUCED CARDIAC PROGENITOR CELLS AND EXOSOMES COMBINATION THERAPIES, Filed Feb. 19, 2019, and is also a CIP of Ser. No. 15/881,693, MICROVESICLE AND STEM CELL COMPOSITIONS FOR THERAPEUTIC APPLICATIONS, Filed Jan. 26, 2018, each incorporated by reference in its entirety for all purposes.

This application is being filed electronically via EFS-Web and includes an electronically submitted sequence listing in .txt format. The .txt file contains a sequence listing entitled “2018-06-12 ISPH_004_ST25.txt” created on Nov. 4, 2020 and is 3,310 bytes in size. The sequence listing contained in this .txt file is part of the specification and is hereby incorporated by reference herein in its entirety. The content of the sequence listing information recorded in computer readable form is identical to the written sequence listing and includes no new matter.

### Related U.S. Patent Document and Granted Patent #

| application No. | Filing Date | U.S. Pat. No. |
|---|---|---|
| 14/255,789 | Apr. 17, 2014 | |
| 15/881,693 | | |
| 14/951,354 | Nov. 24, 2015 | |
| 14/255,789 | | |
| 15/201,292 | Jul. 1, 2016 | 10/443,044 |
| 14/951,354 | | |

### FEDERALLY SPONSORED RESEARCH STATEMENT

This invention was made with government support under Grant Nos: RO1 HL126516, HL134354 and RO1 AR070029 awarded by the National Institutes of Health. The government has certain rights in the invention.

### FIELD OF THE DISCLOSURE

The present disclosure combines induced cardiac progenitor cells (“CPC”) plus additional CPC exosomes (beyond what might be naturally present therein) as treatment compositions, methods of making, and their use in various combination therapies.

### Definitions

The practice of the present invention will employ, unless otherwise indicated, conventional techniques of tissue culture, immunology, molecular biology, microbiology, cell biology and recombinant DNA, which are within the skill of the art. See, e.g., Sambrook, Fritsch and Maniatis (1989) Molecular Cloning: A Laboratory Manual, 2^{nd} edition; F. M. Ausubel, et al. eds. (1987) Current Protocols In Molecular Biology; the series Methods in Enzymology (Academic Press, Inc.): PCR 2: A Practical Approach (1995) (M. J. MacPherson, B. D. Hames and G. R. Taylor eds.); Harlow and Lane, eds. (1988) Antibodies, A Laboratory Manual; Harlow and Lane, eds. (1999) Using Antibodies, A Laboratory Manual; and R. I. Freshney, ed. (1987) Animal Cell Culture.

Numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a,” “an” and “the” include plural references unless the context clearly dictates otherwise.

The terms autologous transfer, autologous transplantation, autograft and the like refer to treatments wherein the cell donor is also the recipient of the cell replacement therapy. The terms allogeneic transfer, allogeneic transplantation, allograft and the like refer to treatments wherein the cell donor is of the same species as the recipient of the cell replacement therapy, but is not the same individual. A cell transfer in which the donor's cells and have been histocompatibly matched with a recipient is sometimes referred to as a syngeneic transfer. The terms xenogeneic transfer, xenogeneic transplantation, xenograft and the like refer to treatments wherein the cell donor is of a different species than the recipient of the cell replacement.

A “biocompatible scaffold” refers to a scaffold or matrix for tissue-engineering purposes with the ability to perform as a substrate that will support the appropriate cellular activity to generate the desired tissue, including the facilitation of molecular and mechanical signaling systems, without eliciting any undesirable effect in those cells or inducing any undesirable local or systemic responses in the eventual host. In other embodiments, a biocompatible scaffold is a precursor to an implantable device which has the ability to perform its intended function, with the desired degree of incorporation in the host, without eliciting an undesirable local or systemic effect in the host. Biocompatible scaffolds are described in U.S. Pat. No. 6,638,369.

As used herein, a “cardiac patch” or “cardiac progenitor patch embedded in fibrin” or “Epicardial patch” is a bioengineered 2D or 3-dimensional (3D) tissue patch comprising or containing iPS cells or iPS cells derived cardiac lineage or cardiac progenitor cells.

A “cardiomyocyte” or “cardiac myocyte” is a specialized muscle cell which primarily forms the myocardium of the heart. Cardiomyocytes have five major components: 1. cell membrane (sarcolemma) and T-tubules, for impulse conduction, 2. sarcoplasmic reticulum, a calcium reservoir needed for contraction, 3. contractile elements, 4. mitochondria, and 5. a nucleus. Cardiomyocytes can be subdivided into subtypes including, but not limited to, atrial cardiomyocyte, ventricular cardiomyocyte, SA nodal cardiomyocyte, peripheral SA nodal cardiomyocyte, or central SA nodal cardiomyocyte. Stem cells can be propagated to mimic the physiological functions of cardiomyocytes or alternatively, differentiate into cardiomyocytes. This differentiation can be detected by the use of markers selected from, but not limited to, myosin heavy chain, myosin light chain, actinin, troponin, tropomyosin, GATA4, Mef2c, and Nkx-2.5.

The cardiomyocyte marker “myosin heavy chain” and “myosin light chain” are part of a large family of motor proteins found in muscle cells responsible for producing contractile force. These proteins have been sequenced and characterized, see for example GenBank Accession Nos. AAD29948, CAC70714, CAC70712, CAA29119, P12883, NP_000248, P13533, CAA37068, ABR18779, AAA59895, AAA59891, AAA59855, AAB91993, AAH31006, NP_000423, and ABC84220. The genes for these proteins has also been sequenced and characterized, see for example GenBank Accession Nos. NM_002472 and NM_000432.

The cardiomyocyte marker “actinin” is a mircrofilament protein which are the thinnest filaments of the cytoskeleton found in the cytoplasm of all eukaryotic cells. Actin polymers also play a role in actomyosin-driven contractile processes and serve as platforms for myosin's ATP hydrolysis-dependent pulling action in muscle contraction. This protein has been sequenced and characterized, see for example GenBank Accession Nos. NP_001093, NP_001095, NP_001094, NP_004915, P35609, NP_598917, NP_112267, AAI07534, and NP_001029807. The gene for this protein has also been sequenced and characterized, see for example GenBank Accession Nos. NM_001102, NM_004924, and NM_001103.

The cardiomyocyte marker “troponin” is a complex of three proteins that is integral to muscle contraction in skeletal and cardiac muscle. Troponin is attached to the protein “tropomyosin” and lies within the groove between actin filaments in muscle tissue. Tropomyosin can be used as a cardiomyocyte marker. These proteins have been sequenced and characterized, see for example GenBank Accession Nos. NP_000354, NP_003272, P19429, NP_001001430, AAB59509, AAA36771, and NP_001018007. The gene for this protein has also been sequenced and characterized, see for example GenBank Accession Nos. NM_000363, NM_152263, and NM_001018007.

“Clonal proliferation” refers to the growth of a population of cells by the continuous division of single cells into two identical daughter cells and/or population of identical cells.

CTGF, also known as CCN2 or connective tissue growth factor, is a matricellular protein of the CCN family of extracellular matrix-associated heparin-binding proteins (see also CCN intercellular signaling protein).

Telomerase reverse transcriptase (“TERT”) is a catalytic subunit of the enzyme telomerase, which, together with the telomerase RNA component (TERC), comprises the most important unit of the telomerase complex.

The miR-290-295 cluster is a pluripotent cluster codes for a family of microRNAs (miRNAs) that are expressed de novo during early embryogenesis and are specific for mouse embryonic stem cells (ESC) and embryonic carcinoma cells (ECC). Such are known in the art and described, for example, in Lichner et al. (2011) Differentiation, Jan. 81(1):11-24.

Chemokine (C-C motif) ligand 7 (CCL7) is a small cytockine previously known as monocyte-specific chemokine 3 (MCP3). The protein sequence is available under Accession number NP_006264 and the murine sequence is available under NP_038682 (see also ncbi.nlm.nih.gov/gene/6354, last accessed on Apr. 16, 2014). An antibody and kit to detect CCL7 is available from Sino Biological Inc.

CXCR2 chemokine receptor 2 (CXCR2) is a protein encoded by this gene is a member of the G-protein-coupled receptor family. This protein is a receptor for interleukin 8 (TL8). It binds to TL8 with high affinity and transduces the signal through a G-protein activated second messenger system. This receptor also binds to chemokine (C-X-C motif) ligand 1 (CXCL1/MGSA). Information regarding the protein and its gene is found on nchbi.nlm.nih.gov/gene/3579 (last accessed on Apr. 16, 2014).

Integral membrane protein 2A is a stem cell marker. The sequence of the human gene is reported at UniProtKB (043736) and the murine sequence is reported at Q61500 (uniprot.org/uiprot, last accessed on Apr. 16, 2014).

DNA (cytosine-5)-methyltransferase 1 is an enzyme that is encoded by the DNMT1 gene. The complete sequence of the protein and its gene is available at genecards.org/cgi-bin/carddisp.pl?gene=DNMT1, last accessed on Apr. 16, 2014. Antibodies to detect the protein are commercially available, e.g., from Cell Signaling Technologies (DNMT1 (D63A6) XP® Rabbit mAb #5032). DNA (cytosine-5)-methyltransferase 3 is an enzyme that is encoded by the DNMT3 gene.

EFNA3 or ephrin A3 is a protein receptor. The human protein sequence is reported at ncbi.nlm.nih.gov/gene/1944. Antibodies useful for the detection and analysis of the protein are available from R&D Systems and Santa Cruz Biotechnology.

“Let-7” refers to a family of microRNAs. The sequences are reported at the miRBase at mirbase.org/cgi-bin/mirna_summary.pl?fam=MIPF000002, last accessed on Apr. 16, 2014. Methods for detecting such are known in the art, e.g., U.S. Patent Application Publication No. 2014/0005251.

Max is a pluripotency marker that binds MYC. See Chappell et al. (2013) Genes & Dev. 27:725-733.

The protein encoded by the Tsg101 gene belongs to a group of apparently inactive homologs of ubiquitin-conjugating enzymes. The gene product contains a coiled-coil domain that interacts with stathmin, a cytosolic phosphoprotein implicated in tumorigenesis. The protein may play a role in cell growth and differentiation and act as a negative growth regulator. In vitro steady-state expression of this tumor susceptibility gene appears to be important for maintenance of genomic stability and cell cycle regulation. Mutations and alternative splicing in this gene occur in high frequency in breast cancer and suggest that defects occur during breast cancer tumorigenesis and/or progression.

CD9 encodes a member of the transmembrane 4 superfamily, also known as the tetraspanin family. Tetraspanins are cell surface glycoproteins with four transmembrane domains that form multimeric complexes with other cell surface proteins. The encoded protein functions in many cellular processes including differentiation, adhesion, and signal transduction, and expression of this gene plays a critical role in the suppression of cancer cell motility and metastasis.

GDF-11 is a gene which also has different alias including Growth Differentiation Factor, Growth/Differentiation Factor, Bone Morphogenetic Protein, BMP-11, Growth differentiation factor 11 also known as bone morphogenetic protein 11 is a protein that in humans is encoded by the growth differentiation factor 11 gene. GDF11 is a member of the of the Transforming growth factor beta family.

ROCK-2 is a gene which also has different alias including Rho Associated Coiled-Coil Containing Protein Kinase, Rho-Associated, Coiled-Coil-Containing Protein Kinase II, Rho-Associated Protein Kinase, P164 ROCK-2, and EC 2.7.11.1. Rho associated coiled-coil containing protein kinase 2 is a protein that in humans is encoded by the ROCK2 gene. The protein encoded by this gene is a serine/threonine kinase that regulates cytokinesis, smooth muscle contraction, the formation of actin stress fibers and focal adhesions, and the activation of the c-fos serum response element. This protein, which is an isozyme of ROCK1 is a target for the small GTPase Rho.

As used herein, the term “microRNAs” or “miRNAs” refers to post-transcriptional regulators that typically bind to complementary sequences in the three prime untranslated regions (3′ UTRs) of target messenger RNA transcripts (mRNAs), usually resulting in gene silencing. Typically, miRNAs are short, non-coding ribonucleic acid (RNA) molecules, for example, 21 or 22 nucleotides long. The terms “microRNA” and “miRNA” are used interchangeably.

mir-373 is annotated as ENSG00000199143 and miRBase: has-mir-373.

mir-210 is annotated as ENSG00000199038 and miRBase: has-mir-210 and is associated with Sudden Infant Death Syndrome susceptibility.

Tcf15 enocodes a basic helix-loop-helix transcription factor expressed early in development, which is involved in patterning of the mesoderm and its derivative cell types. This gene is annotated as Ensembl: ENSG00000125878 and Uniprot Q12870.

mir-377 is annotated as ENSG00000199015 and miRBase: has-mir-377.

mir-367 is annotated as ENSG00000199169 and miRBase: has-mir-367.

mir-520c is annotated as ENSG00000207738 and miRBase: has-mir-520c.

mir-548ah is annotated as ENSG00000283682 and miRBase: has-mir-548ah.

Dystrophin intends a protein encoded by the gene Dmd, annotated as Ensembl: ENSG00000198947 and Uniprot: P11523. This protein is a component of the dystrophin-glycoprotein complex, which anchors the cytoskeleton to the extra-cellular matrix. Mutations are associated with Duchenne muscular dystrophy, Becker muscular dystrophy and cardiomyopathy, as well as equivalents thereof.

mir-548q is annotated as ENSG00000221331 and miRBase: has-mir-548q.

mir-548q is annotated as ENSG00000221331 and miRBase: has-mir-548q.

mir-335 encodes microRNA-335, annotated as ENSG00000199043 and miRBase: has-mir-335.

mir-21 encodes microRNA-21, annotated as ENSG00000284190 and miRBase: has-mir-21. This miRNA is expressed in stem cells and plays a role in cancer.

mir-30c1 encodes a microRNA annotated as Ensembl: ENSG00000207962 and miRBase: has-mir-30c-1, which may be involved in ECM maintenance and cancer.

mir-30c2 encodes a microRNA annotated as Ensembl: ENSG00000199094 and miRBase: has-mir-30c-2. Similar to miR-30c1, it may be involved in ECM maintenance and cancer.

Meox1 encodes the mesenchyme homeobox 1 protein, and is annotated as Ensembl: ENSG00000005102 and Uniprot: P50221. This protein plays a role in somite development. Genetic mutations are associated with Klippel-Feil Syndrome.

Meox2 encodes the mesenchyme homeobox 2 protein, and is annotated as Ensemble: ENSG00000106511 and Uniprot: P50222. Based on homology to the mouse, this protein is thought to play a role in myogenesis and limb development. Mutations are associated with craniofacial and skeletal abnormalities as well as Alzheimer's.

Pax3 is annotated as Ensembl: ENSG00000135903 and Uniprot: P23760. This gene encodes a member of the paired box family of transcription factors, which regulates proliferation and migration during neural development and myogenesis. Mutations in this gene are associated with craniofacial-deafness-hand syndrome, Rhabdomyosarcoma, and Waardenburg syndrome.

Pax7 is annotated as Ensembl: ENSG00000009709 and Uniprot: P23759. This gene encodes a member of the paired box family of transcription factors, which regulates proliferation of muscle precursor cells. It is vital for embryonic development and implicated in cancer, including Rhabdomyosarcoma.

MyoD1 is annotated as Ensembl: ENSG00000129152 and Uniprot: P15172. This gene encodes a myogenic helix-loop-helix transcription factor that regulates myocyte differentiation via inhibition of the cell cycle. This protein is known to interact with other key muscle factors, Myf5, Myf6, and MyoG.

MyoG encodes the muscle-specific basic helix-loop-helix transcription activator, myogenin.

Myh2 encodes a class II or conventional myosin heavy chain. As a motor protein, it functions in skeletal muscle contraction, and mutations are associated with inclusion-body myopathy. Myh2 is annotated as Ensembl: ENSG00000125414 and Uniprot: Q9UKX2. Numerous splice variants have been reported.

Myh6 encodes a motor protein that forms the alpha heavy chain subunit of cardiac myosin. This gene is annotated as Ensemble: ENSG00000197616 and Uniprot: P13533, and mutations are associated with atrial septal defects and hypertrophic cardiomyopathy.

Tbx1 encodes a member of the developmentally important T-box transcription factor family. It is annotated as Ensembl: ENSG00000184058 and Uniprot: Q43435. Mutations in this gene are associated with neural-crest defects, DiGeorge syndrome, and velocardiofacial syndrome.

Mesp1 encodes a basic helix-loop-helix transcription factor that is involved in development of the somatic and cardiac mesoderm, and rostrocaudal patterning of the somites. Mesp1 is annotated in Ensembl: ENSG00000166823 and Uniprot: QOBRJ9.

Des encodes the protein Desmin and is annotated as Ensemble: ENSG00000175084 and Uniprot: P17661. Desmin is a muscle-specific class III intermediate filament that forms a fibrous network for myofibrils. Mutations in Des are associated with cardiac and skeletal muscle myopathies.

Cnntb1 is a well-known gene that encodes the protein, β-catenin, a key component of the canonical Wnt signaling pathway. In the presence of Wnt, β-catenin translocates to the nucleus as acts as a transcriptional regulator. This protein is also involved in regulation of contact inhibition. Mutations in this gene are associated with mental retardation and colorectal cancer. The Cnntb1 gene is annotated as Ensemble: ENSG00000168036 and Uniprot: P35222.

Pax7 is annotated as Ensembl: ENSG00000009709 and Uniprot: P23759. This gene encodes a member of the paired box family of transcription factors, which regulates proliferation of muscle precursor cells. It is vital for embryonic development and implicated in cancer, including Rhabdomyosarcoma.

Myf5 gene, Ensemble: ENSG00000111049, encodes a master transcriptional regulator of muscle differentiation, that binds and promotes transcription of numerous myogenic factors (Uniprot: P13349). Mutations in Myf5 are associated with skeletal muscle cancer and Rhabdomyosarcoma.

MyoD1 is annotated as Ensembl: ENSG00000129152 and Uniprot: P15172. This gene encodes a myogenic helix-loop-helix transcription factor that regulates myocyte differentiation via inhibition of the cell cycle. This protein is known to interact with other key muscle factors, Myf5, Myf6, and MyoG.

xESI myogenic genes intend Myogenic regulatory factors (MRF) which are basic helix-loop-helix (bHLH) transcription factors that regulate myogenesis: MyoD, Myf5, myogenin, and MRF4. These proteins contain a conserved basic DNA binding domain that binds the E box DNA motif.[2] They dimerize with other HLH containing proteins through an HLH-HLH interaction.

Pitx2 is annotated as ENSG00000164093 and Uniprot: Q99697. This gene encodes Paired-like homeodomain transcription factor 2, which belongs to the bicoid family of homeodomain proteins. This protein regulates the hormone, Prolactin, and is important for development of eyes, teeth, and abdominal organs. Mutations in this gene associate with Axenfeld-Rieger Syndrome.

ISL1 is a gene (Ensembl: ENSG00000016082) that encodes the transcription factor, ISL LIM homeobox 1 (Uniprot: P61371). This protein is implicated in motor neuron and retinal ganglion cell specification and regulating expression of the Insulin gene. Mutations are associated with maturity-onset diabetes and bladder exstrophy.

Nkx2.5 encodes a master transcription factor involved in cardiac development. Annotated as Ensembl: ENSG00000183072 and Uniprot P52952, mutations this gene can result in atrial septal defects, and a form of congenital hypothyroidism.

Hand1 is a basic helix-loop-helix transcription factor annotated as Ensembl: ENSG00000113196 and Uniprot: Q96004. During heart development, Hand1 is expressed asymmetrically with another Hand protein to direct cardiac morphogenesis and formation of the right ventricle and aortic arch arteries. Mutations in genes encoding Hand proteins are associated with congenital heart disease.

GATA4 is annotated as ENSG000001366574 in Ensembl, and encodes a member of the gata family of zinc-finger transcription factors. This protein, Uniprot: P43694, is key to embryogenesis, cardiac development, and myocardial function. Mutations are associated with septal defects and various forms of cancer.

Tbx5 is a member of the T-box gene family, which contains a conserved DNA-binding domain. Numerous transcripts of Tbx5 are curated in RefSeq and the Ensembl gene identifier is ENSG00000089225. The protein product, Uniprot: Q99593, is important for heart and limb development, and mutations in this gene are associated with Holt-Oram syndrome.

TnnT2 is a gene encoding cardiac troponin T2 (Uniprot:P45379) the tropomyosin-binding unit of the troponin complex. In response to changes in intracellular calcium levels, Tnnt2 regulates muscle contraction. Mutations in this gene, annotated as Ensembl: ENSG00000118194, are associated with familiar hypertrophic cardiomyopathy and dilated cardiomyopathy.

Myl7 is a gene encoding the calcium binding motor protein myosin light chain 7. This gene is annotated in Ensembl: ENSG00000106631 and UniProt: Q01449. Mutation at this locus are associated with Fechtner Syndrome and Familial Atrial Fibrillation.

MLC2v gene (more commonly denoted as Myl2, in humans), curated as Refseq: NM_00432 and Uniprot: P10916, encodes the motor protein myosin light chain 2. Calcium dependent phosphorylation of this protein results in generation of contractile forces. This protein functions in heart development and cardiac contractility, and mutations are associated with mid-left ventricular chamber hypertrophic cardiomyopathy. Antibodies are available through Invitrogen and Santa Cruz Biotechnology.

Mef2c is a gene (Ensembl ID ENSG00000081189) that produces more than 8 alternatively spliced transcripts curated in RefSeq. The protein product (Uniprot: Q06413) is a member of the MADS box transcription enhancer factor 2 family, and plays a role in vascular development, cardiac morphogenesis, myogenesis, and maintenance of the differentiated state. Genomic aberrations within this gene locus are associated with mental retardation, cerebral malformation, epilepsy, and arrhythmogenic right ventricular dysplasia 5. Cell Signaling Technologies, Novus Biologicals, and Invitrogen all provides products for detection and study of this protein.

Cdh4 gene produces three transcript variants encoding the protein, Cadherin 4. A member of the cadherin superfamily, Chd4 functions as a calcium-dependent cell adhesion molecule important for brain segmentation and neuronal outgrowth. This protein is also implicated in kidney and muscle development. Cdh4 is annotated in Refseq: NM_001252399 and Uniprot: P55283. Purified protein, antibodies, and other detection kits are widely available through sources including Invitrogen, Abcam, and R&D systems.

Lhx2 encodes the Lim homeobox 2 protein, a member of the LIM domain family, which carry a cysteine-rich zinc binding domain. Lhx2 is curated in Refseq: NM_004789 and UniProt: P50458, and believed to function as a transcriptional activator involved in cellular differentiation and development of the lymphoid and neural lineages. Antibodies and ELISA detection kits for Lhx2 are commercially available from Origene, Santa Cruz Biotechnology and Invitrogen

Gαi is a heterotrimeric G protein subunit that inhibits the product of cAMP from ATP. An exemplary sequence is provided under GenBank Ref.: NM_002069 and UnProt P63096. Antibodies that recognize this marker are commercially available from Santa Cruz Biotechnology.

Cytoskeletal remodeling intends remodeling intends the dynamic reorganization of microfilaments (actins), microtubules (tubulin), and intermediate filaments (i.e. vimentin, keratin, desmin), which comprise the eukaryotic cytoskeleton. Though complex, this process occurs within minutes and facilitates biological functions such as cell migration, cytokinesis, and muscle contraction.

Promoting TGF-β induced emt(epithelial-mensenchymal transition) signaling intends transdifferentiation of cells with epithelial-like properties into cells with mesenchymal-like properties, as mediated by the signaling molecule TGF-β. Non-limiting biological roles for this process, referred to as EMT, include cancer, fibrosis, heart development, and cardiac differentiation. Transforming growth factor-β (TGF-β) is a potent inducer of EMT both during development and in cancer. In TGF-β induced EMT, activation of Smad proteins results in their nuclear translocation, DNA binding, and upregulation of EMT transcriptionfactors. Non-limiting examples of EMT transcription factors include Snail, Twist, and Zeb. EMT requires cytoskeletal remodeling and cardiac differentiation intends efficient differentiation of human pluripotent stem cells (PSCs) such a IPS cells to contracting cardiomyocytes.

Promoting expression of genes for development of pip3 signaling in cardiomyocytes, muscle contraction and nf-at hypertrophy signaling pathways intends activate downstream signaling components, the most notable one being the protein kinase AKT, which activates downstream anabolic signaling pathways required for cell growth and survival. Phosphatidylinositol (3,4,5)-trisphosphate (PtdIns(3,4,5)P3), abbreviated PIP3, is the product of the class I phosphoinositide 3-kinases (PI 3-kinases) phosphorylation of phosphatidylinositol (4,5)-bisphosphate (PIP2). It is a phospholipid that resides on the plasma membrane. PIP3 signaling in cardiac myocytes.

Phosphoinositide 3-kinase (PI3K) can be activated in cardiac myocytes by the receptors with intrinsic tyrosine kinase activity, such as insulin receptor (INSR), growth factor receptors (IGF1 receptor and HGF receptor), and by the G protein-coupled receptors (GPCRs). INSR and IGF1 receptor engagement triggers receptor activation and autophosphorylation. The activated receptor can then phosphorylate several intracellular protein substrates, most notably the insulin receptor substrate (IRS1-4) proteins. Tyrosine-phosphorylated IRS1 can recruit and activate the downstream effector, PI3K, which generates phosphatidylinositol 3,4,5-trisphosphate (PIP3) using inositol-containing phospholipids resident in the plasma membrane as substrates. IRS proteins also recruit adaptors Shc and Grb-2. The protein tyrosine phosphatase PTP1B is responsible for negatively regulating INSR signaling by dephosphorylating the phosphotyrosine residues of this receptor. Hepatocyte growth factor receptor (HGF receptor) activation induces the tyrosine phosphorylation of GAB1 and its association with PI3K via the recruitment of its regulatory subunit (PI3KR class 1A) that stimulates its catalytic subunit (PI3KC class 1A). Activated adaptors Shc and Grb-2 recruit exchange factor SOS that activates H-RAS [4]. H-RAS directly stimulates PI3K catalytic subunit (PI3KC class 1A). PI3K converts phosphatidylinositol 4,5-biphosphate (PI(4,5)P2) to PIP3 [6]. PIP3 is the second messenger that activates diverse signal cascades, including PDK and AKT pathway. Phosphatase PTEN acts as a negative regulator for the PI3K/AKT signaling pathway, converting PI(3,4,5)P3 into PI(4,5)P2. AKT and PDK phosphorylate diverse proteins that mediate various insulin- and growth factor-induced cellular responses such as glycogen synthesis, protein synthesis, cell cycle initiation, and promotion of cell survival by regulation of apoptosis factors such as BAD and Bcl-x(L).

As used herein, the term “microRNAs” or “miRNAs” refers to post-transcriptional regulators that typically bind to complementary sequences in the three prime untranslated regions (3′ UTRs) of target messenger RNA transcripts (mRNAs), usually resulting in gene silencing. Typically, miRNAs are short, non-coding ribonucleic acid (RNA) molecules, for example, 21 or 22 nucleotides long. The terms “microRNA” and “miRNA” are used interchangeably.

miR-133 refers to a microRNA that has been linked to an immature or undifferentiated phenotype. Methods to detect such include, for example, microarray-RT-PCR and RNA-seq. Commercially available kits to miR-133 is available from EMD Millipore (SmartFlare™ Detection Probes) which allow for the detection of miRNA in live cells.

miR-762 is a non-coding RNA that has been linked to post-transcriptional regulation of gene expression in multicellular organisms. The miR-762 human sequence is reported under Accession No. MI0003892 (last accessed on Apr. 16, 2014). The murine sequence is reported under NR_030428.1 (see ncbi.nlm.nih.gov/gene/79103, last accessed on Apr. 16, 2014). Methods to detect such are known in the art and kits are commercially available from, for example, Origene (miR-762, see origene.com, last accessed on Apr. 16, 2014).

miR-133 refers to a microRNA that has been linked to an immature or undifferentiated phenotype. Methods to detect such include, for example, microarray-RT-PCR and RNA-seq. Commercially available kits to miR-133 is available from EMD Millipore (SmartFlare™ Detection Probes) which allow for the detection of miRNA in live cells.

miR-762 is a non-coding RNA that has been linked to post-transcriptional regulation of gene expression in multicellular organisms. The miR-762 human sequence is reported under Accession No. MI0003892 (last accessed on Apr. 16, 2014). The murine sequence is reported under NR_030428.1 (see ncbi.nlm.nih.gov/gene/79103, last accessed on Apr. 16, 2014). Methods to detect such are known in the art and kits are commercially available from, for example, Origene (miR-762, see origene.com, last accessed on Apr. 16, 2014).

miR-195 is an RNA gene and is reported to be affiliated with the miRNA class. Diseases associated with miR-195 include tongue squamous cell carcinoma and primary peritoneal carcinoma. Among its related pathways are microRNAs in cancer and microRNAs in cardiomyocyte hypertrophy. It is also known as MIRN195, Has-MIR-195 and MiRNA 195. The sequence and homologs are reported in the genecards web page. Nucleic acid sequences are reported under GenBank Accession No. AK098506, last accessed on Nov. 18, 2015.

ILS 1 refers to an insulin gene enhancer protein, which plays an important role in regulating insulin gene expression. ISL1 is also found central to the development of pancreatic cell lineages and may also be required for motor neuron generation. ISL1 is identified as a marker for cardiac progenitor cells.

Tbx-5 is a cardiac transcription factor, also known as T-box transcription factor (“TBX5”) is a protein that in humans is encoded by the TBX5 gene. As indicated on the GeneCards human gene database, this gene is a member of a phylogenetically conserved family of genes that share a common DNA-binding domain, the T-box. T-box genes encode transcription factors involved in the regulation of developmental processes. This gene is closely linked to related family member T-box 3 (ulnar mammary syndrome) on human chromosome 12. The encoded protein may play a role in heart development and specification of limb identity. Mutations in this gene have been associated with Holt-Oram syndrome, a developmental disorder affecting the heart and upper limbs. Several transcript variants encoding different isoforms have been described for this gene. The accession for the protein is Q99593 or alternatively A6ND77, or alternatively 015301, or alternatively Q96TBO. Antibodies to the protein are commercially available from R&D Systems, Browse EMD, OriGene Antibodies, and Novus Biologicals.

As used herein, the term “a protein that facilitates regeneration and/or improves function of a tissue” intends a protein that can either regenerate or regrow or improve the tissue function or bone function is a potential cytokine which improves tissue regeneration or bone function. The gel-forming property makes certain protein polymer highly suitable for biomedical applications, such as tissue regeneration in operations and wounds. Non-limiting examples of such include IGFBP5 protein which enhances periodontal tissue and PPARα which activates liver regeneration.

As used herein, “lyophilization” intends low temperature drying or freeze drying.

Cell-derived exosomes or microvesicles, also referred to as extracellular exosomes or microvesicles, are membrane surrounded structures that are released by cells in vitro and in vivo. Extracellular exosomes or microvesicles can contain proteins, lipids, and nucleic acids and can mediate intercellular communication between different cells, including different cell types, in the body. Two types of extracellular exosomes or microvesicles are exosomes or microvesicles and microvesicles. Exosomes or microvesicles are small lipid-bound, cellularly secreted exosomes or microvesicles that mediate intercellular communication via cell-to-cell transport of proteins and RNA (El Andaloussi, S. et al. (2013) Nature Reviews: Drug Discovery 12(5):347-357). Exosomes or microvesicles range in size from approximately 30 nm to about 200 nm. Exosomes or microvesicles are released from a cell by fusion of multivesicular endosomes (MVE) with the plasma membrane. Microvescicles, on the other hand, are released from a cell upon direct budding from the plasma membrane (PM) and are packaged with different factors. Microvesicles are typically larger than exosomes or microvesicles and range from approximately 200 nm to 1 μm and have different functionalities.

Cell-derived exosomes or microvesicles can be isolated from eukaryotic cells using commercially available kits as disclosed herein and available from biovision.com and novusbio.com, or using the methods described herein. Non-limiting examples of cells that cell-derived exosomes or microvesicles can be isolated from include stem cells. Non-limiting examples of such stem cells include adult stem cells, embryonic stem cells, embryonic-like stem cells, non-embryonic stem cells, or induced pluripotent stem cells.

As used herein, the terms “overexpress,” “overexpression,” and the like are intended to encompass increasing the expression of a nucleic acid or a protein to a level greater than the exosome or microvesicle naturally contains. It is intended that the term encompass overexpression of endogenous, as well as heterologous nucleic acids and proteins.

As used herein, the term “homogeneous” in reference to a population of e cell-derived exosomes or microvesicles refers to population of cell-derived exosomes or microvesicles that have a similar amount of an exogenous nucleic acid, a similar amount of an exogenous protein, are of a similar size, or combinations thereof. A homogenous population is one wherein about 65%, about 70%, about 75%, about 80%, about 85%, about 90%, about 95%, about 98%, or 100% of the cell-derived exosomes or microvesicles share at least one characteristic. Another example of a homogenous population is one wherein about 90% of the exosomes or microvesicles are less than 50 nm in diameter.

As used herein, the term “heterogeneous” in reference to a population of cell-derived exosomes or microvesicles refers to population of cell-derived exosomes or microvesicles that have differing amounts of an exogenous nucleic acid, differing amounts of an exogenous protein, are of a different size, or combinations thereof.

The term “substantially” refers to the complete or nearly complete extent or degree of a characteristic and in some aspects, defines the purity of the isolated or purified population of exosomes or microvesicles.

The term “purified population,” relative to cell populations, cell-derived exosomes or microvesicles or miRNA, as used herein refers to plurality of such that have undergone one or more processes of selection for the enrichment or isolation of the desired exosome or microvesicle or miRNA population relative to some or all of some other component with which cell-derived exosomes or microvesicles are normally found in culture media. Alternatively, “purified” can refer to the removal or reduction of residual undesired components found in the conditioned media (e.g., cell debris, soluble proteins, etc.). A “highly purified population” as used herein, refers to a population of cell-derived exosomes or microvesicles in which at least 65%, at least 70%, at least 75%, at least 80%, at least 85%, at least 90%, at least 95%, at least 98%, at least 99% or 100% of cell debris and soluble proteins (e.g., proteins derived from fetal bovine serum and the like) in the conditioned media along with the cell-derived exosomes or microvesicles or miRNA are removed. The cells, populations, exosomes or microvesicles and miRNA as described herein can be provided in isolated, purified, highly purified forms, homogeneous, substantially homogeneous and heterogenous forms.

As used herein the terms “culture media” and “culture medium” are used interchangeably and refer to a solid or a liquid substance used to support the growth of cells (e.g., stem cells). Preferably, the culture media as used herein refers to a liquid substance capable of maintaining stem cells in an undifferentiated state. The culture media can be a water-based media which includes a combination of ingredients such as salts, nutrients, minerals, vitamins, amino acids, nucleic acids, proteins such as cytokines, growth factors and hormones, all of which are needed for cell proliferation and are capable of maintaining stem cells in an undifferentiated state. For example, a culture media can be a synthetic culture media such as, for example, minimum essential media α (MEM-α) (HyClone Thermo Scientific, Waltham, Mass., USA), DMEM/F12, GlutaMAX (Life Technologies, Carlsbad, Calif., USA), Neurobasal Medium (Life Technologies, Carlsbad, Calif., USA), KO-DMEM (Life Technologies, Carlsbad, Calif., USA), DMEM/F12 (Life Technologies, Carlsbad, Calif., USA), supplemented with the necessary additives as is further described herein. In some embodiments, the cell culture media can be a mixture of culture media. Preferably, all ingredients included in the culture media of the present disclosure are substantially pure and tissue culture grade. “Conditioned medium” and “conditioned culture medium” are used interchangeably and refer to culture medium that cells have been cultured in for a period of time and wherein the cells release/secrete components (e.g., proteins, cytokines, chemicals, etc.) into the medium.

A “composition” is also intended to encompass a combination of a cell, a cell population, an exosome or microvesicle, an miRNA, or populations of such, or an active agent, and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include biocompatible scaffolds, pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

Preservative intends a composition that enhances the viability of an agent in a composition. Non-limiting examples include Benzoates (such as sodium benzoate, benzoic acid), Nitrites (such as sodium nitrite) and Sulphites (such as sulphur dioxide).

A cryoprotective is a compound that protects the agent during freezing and thawing procedures. Non-limiting examples of such include DMSO, Glycerol, PEG.

A “control” is an alternative subject or sample used in an experiment for comparison purpose. A control can be “positive” or “negative”. For example, where the purpose of the experiment is to determine a correlation of an altered expression level of a gene with a particular phenotype, it is generally preferable to use a positive control (a sample from a subject, carrying such alteration and exhibiting the desired phenotype), and a negative control (a subject or a sample from a subject lacking the altered expression or phenotype). Additionally, when the purpose of the experiment is to determine if an agent effects the differentiation of a stem cell or expression of an exosome or microvesicle or miRNA, it is preferable to use a positive control (a sample with an aspect that is known to affect differentiation or altered expression) and a negative control (an agent known to not have an affect or a sample with no agent added).

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

As used herein, the term “detectably labeled” means that the agent (biologic or small molecule) is attached to another molecule, compound or polymer that facilitates detection of the presence of the agent in vitro or in vivo.

A “detectable label” intends a directly or indirectly detectable compound or composition that is conjugated directly or indirectly to the composition to be detected, e.g., N-terminal histadine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small-scale detection or more suitable for high-throughput screening. As such, suitable labels include, but are not limited to magnetically active isotopes, non-radioactive isotopes, radioisotopes, fluorochromes, luminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue®, and Texas Red. Other suitable optical dyes are described in the Haugland, Richard P. (1996) Handbook of Fluorescent Probes and Research Chemicals (6^{th} ed.).

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

“Differentially expressed” intends an up- or downward expression of a gene, exosome or microvesicle, or marker, for example, as compared to a control. In one aspect, a control is a differentiated cell as compared to a pluripotent or stem cell. “Differentially expressed” as applied to a gene, protein, cell, population, exosome or microvesicle, miRNA, or marker, refers to the differential production of the product as compared to a control such as expression level found in the native environment. Differently expressed is mRNA transcribed from the gene or the protein product encoded by the gene. A differentially expressed gene may be overexpressed or underexpressed (a.k.a. inhibited) as compared to the expression level of a normal, non-treated, native or control cell. In one aspect, it refers to overexpression that is 1.5 times, or alternatively, 2 times, or alternatively, at least 2.5 times, or alternatively, at least 3.0 times, or alternatively, at least 3.5 times, or alternatively, at least 4.0 times, or alternatively, at least 5 times, or alternatively 10 times higher (i.e., and therefore overexpressed) or lower than the expression level detected in a control sample. The term “differentially expressed” also refers to nucleotide sequences in a cell or tissue which are expressed where silent in a control cell or not expressed where expressed in a control cell.

The term “stem cell” refers to a cell that is in an undifferentiated or partially differentiated state and has the capacity for self-renewal and/or to generate differentiated progeny. Self-renewal is defined as the capability of a stem cell to proliferate and give rise to more such stem cells, while maintaining its developmental potential (i.e., totipotent, pluripotent, multipotent, etc.). The term “somatic stem cell” is used herein to refer to any stem cell derived from non-embryonic tissue, including fetal, juvenile, and adult tissue. Natural somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, mesenchymal stem cells (MSCs) and neural stem cells (NSCs). In some embodiments, the stem or progenitor cells can be embryonic stem cells. As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are pluripotent cells derived from the early embryo or blastocyst. Embryonic stem cells can be obtained directly from suitable tissue, including, but not limited to human tissue, or from established embryonic cell lines. “Embryonic-like stem cells” refer to cells that share one or more, but not all characteristics, of an embryonic stem cell.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

As used herein, the term “differentiates or differentiated” defines a cell that takes on a more committed (“differentiated”) position within the lineage of a cell. “Dedifferentiated” defines a cell that reverts to a less committed position within the lineage of a cell. Induced pluripotent stem cells are examples of dedifferentiated cells.

As used herein, the “lineage” of a cell defines the heredity of the cell, i.e. its predecessors and progeny. The lineage of a cell places the cell within a hereditary scheme of development and differentiation.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin yet give rise to different tissues). Another example is a neurogenic cell (of ectodermal origin) and adipogenic cell (of mesodermal origin).

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. An example of progenitor cell includes, without limitation, a progenitor nerve cell.

A “parthenogenetic stem cell” refers to a stem cell arising from parthenogenetic activation of an egg. Methods of creating a parthenogenetic stem cell are known in the art. See, for example, Cibelli et al. (2002) Science 295(5556):819 and Vrana et al. (2003) Proc. Natl. Acad. Sci. USA 100(Suppl. 1)11911-6.

As used herein, a “pluripotent cell” defines a less differentiated cell that can give rise to at least two distinct (genotypically and/or phenotypically) further differentiated progeny cells. In another aspect, a “pluripotent cell” includes an Induced Pluripotent Stem Cell (iPSC) which is an artificially derived stem cell from a non-pluripotent cell, typically an adult somatic cell, that has historically been produced by inducing expression of one or more stem cell specific genes. Such stem cell specific genes include, but are not limited to, the family of octamer transcription factors, i.e. Oct-3/4; the family of Sox genes, i.e., Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e., OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007.

“Embryoid bodies or EBs” are three-dimensional (3D) aggregates of embryonic stem cells formed during culture that facilitate subsequent differentiation. When grown in suspension culture, EBs cells form small aggregates of cells surrounded by an outer layer of visceral endoderm. Upon growth and differentiation, EBs develop into cystic embryoid bodies with fluid-filled cavities and an inner layer of ectoderm-like cells.

An “induced pluripotent cell” intends embryonic-like cells reprogrammed to the immature phenotype from adult cells. Various methods are known in the art, e.g., “A simple new way to induce pluripotency: Acid.” Nature, 29 Jan. 2014 and available at sciencedaily.com/releases/2014/01/140129184445, last accessed on Feb. 5, 2014 and U.S. Patent Application Publication No. 2010/0041054. Human iPSCs also express stem cell markers and are capable of generating cells characteristic of all three germ layers.

As used herein, the term “a cardiac progenitor” intends a dynamic progenitor that is able to differentiate into terminally derived cardiac cell types. Cardiac progenitor cells (CPCs) represent the earliest stages of mesodermal commitment to the cardiac lineage and show a classical CPC marker pro le of KDR/PDGFR-αpos/CKITneg and are responsive to permissive conditions for proliferation as a progenitor population and/or differentiation into terminal cardiac cell.

As used herein, the term “a skeletal myogenic progenitor” intends cells which are characterized by the expression of Pax3 and Pax7 and also give rise to the satellite cells of postnatal muscle.

As used herein, a “fibroblast” intends a cell expressing the following markers Vimentin, CollA1, FSP-1.

As used herein, a “skeletal myoblast” intends a cell expressing the following markers MyoG, Desmin, m-calpain, human alpha-skeletal actin.

As used herein, a “pluripotent cell” defines a less differentiated cell that can give rise to at least two distinct (genotypically and/or phenotypically) further differentiated progeny cells.

A juvenile or young stem cell intends from 2 months or younger mice possessing antiaging genes. In vitro, the cells average from about 3-5 μm in size and express the embryonic stem cell marker, OCT4 and surface markers, CD29, CD44, and CD90.

As used herein, a “fibroblast” intends a cell expressing the following markers Vimentin, CollA1, FSP-1.

As used herein, a “skeletal myoblast” intends a cell expressing the following markers MyoG, Desmin, m-calpain, human alpha-skeletal actin.

As used herein, the term “pluripotent gene or marker” intends an expressed gene or protein that has been correlated with an immature or undifferentiated phenotype, e.g., Oct ¾, Sox2, Nanog, c-Myc and LIN-28. Methods to identify such are known in the art and systems to identify such are commercially available from, for example, EMD Millipore (MILLIPLEX® Map Kit).

A “skeletal myoblast (SM)” is an immature cell that can be isolated from between the basal lamina and sarcolemma. They account for 2-5% of sub-laminar nuclei of mature skeletal muscle. Skeletal myoblasts are activated in response to muscle damage or disease-induced muscle degeneration. Skeletal myoblasts express desmin, CD56, Pax3, Pax7, c-met, myocyte nuclear factor, M-cadherin, VCAM1, N-CAM, CD34, Leu-19, and syndecan 3 and 4. Activated skeletal myoblasts first express Myf-5 and/or MyoD, and finally myogenin and MRF4 as the cells differentiate into multinucleated myotubes.

As used herein, the term “small juvenile stem cells (SJSCs)” intends stem cells isolated from aged bone marrow-derived stem cells (BMSCs) with high proliferation and differentiation potential. See Igura et al. (2013) 305(8):H1354-62. SJSCs express mesenchymal stem cell markers, CD29(+)/CD44(+)/CD59(+)/CD90(+) but are negative for CD45(−)/CD117(−) as examined by flow cytometry analysis. SJSCs show higher proliferation, colony formation, and differentiation abilities compared with BMSCs. They also are reported to significantly express cardiac lineage markers (Gata-4 and myocyte-specific enhancer factor 2C) and pluripotency markers (octamer-binding transcription factor 4, sex-determining region Y box 2, stage-specific embryonic antigen 1, and Nanog) as well as antiaging factors such as telomerase reverse transcriptase and sirtuin 1.

A “marrow stromal cell” also referred to as “a bone marrow stromal cell” or a “mesenchymal stromal cell” is a multipotent stem cell that can differentiate into a variety of cell types. Cell types that MSCs have been shown to differentiate into in vitro or in vivo include osteoblasts, chondrocytes, myocytes, and adipocytes. Mesenchyme is embryonic connective tissue that is derived from the mesoderm and that differentiates into hematopoietic and connective tissue, whereas MSCs do not differentiate into hematopoietic cells. Stromal cells are connective tissue cells that form the supportive structure in which the functional cells of the tissue reside. While this is an accurate description for one function of MSCs, the term fails to convey the relatively recently discovered roles of MSCs in repair of tissue. Methods to isolate such cells, propagate and differentiate such cells are known in the technical and patent literature, e.g., U.S. Patent Application Publication Nos. 2007/0224171, 2007/0054399, 2009/0010895, which are incorporated by reference in their entireties.

Adipose stem cells are also known as adipose tissue-derived stem cells (ADSC) that are routinely isolated from the stromal vascular fraction (SVF) of homogenized adipose tissue. Similar to other types of mesenchymal stem cells (MSC), ADSC remain difficult to define due to the lack of definitive cellular markers. Adipose-derived stem cells (ASCs) are a mesenchymal stem cell source with self-renewal property and multipotential differentiation.

Hematopoietic stem cells are defined as a stem cell that gives rise to all red and white blood cells and platelets. They are commonly isolated by use of the markers CD34+. In another aspect, the hematopoietic stem cell is an adult stem cell comprising the marker profile of: CD34⁺ and/or CD34⁺/Thy-1⁻ HSC). See also Andrews, R. G. et al. (1990) J. Exp. Med. 172(1):355-358, incorporated herein by reference.

Mesenchymal stem cells, or MSCs, are defined as multipotent stromal cells that can differentiate into a variety of cell types, including: osteoblasts (bone cells), chondrocytes (cartilage cells), myocytes (muscle cells) and adipocytes (fat cells).

As used herein, the term chemically induced or chemically modified pluripotent stem cell (iPSC) is intended to include an iPSC treated with a small molecule such as isoxazole or a derivative thereof, or an isoxazole similar molecule.

“Isoxazole” is a class of compounds found in some natural products, such as ibotenic acid, as well as a number of drugs, including a COX-2 inhibitor, and furoxan, a nitric oxide donor. Isoxazoles are useful isosteres of pyridine, and have been found to inhibit voltage-gated sodium channels to control pain, enable the construction of tetracycline antibiotic derivatives, and as treatments for depression. Compounds of this class available from Sigma-Aldrich and methods to synthesize such are known in the art as described for example in U.S. Pat. Nos. 5,059,614 and 8,318,951 and PCT Publication No. WO 1999/002507. Structurally, isoxazole is a five membered heterocyclic compound containing oxygen and nitrogen atoms in the 1, 2 positions. Its partially saturated analogs are called isoxazolines and completely saturated analog is isoxazolidine. Examples of isoxazole-like compounds include derivatives, non-limiting examples of such include sulfamethoxazole, sulfisoxazole, oxacillin, cycloserine and acivicin. isoxazoles, isoxazolines and isoxazolidines may be considered as useful synthons in organic synthesis. Isoxazoles may be efficiently transformed in to various classes of medicinally important molecules. For example, Anthracen-9-ylmethylene-(3,4-dimethylisoxazol-5-yl) amine may be synthesized in high yield by reaction of anthracene-9-carbaldehyde and 5-amino-3,4-dimethylisoxazole in ethanol. In an embodiment, all the derivatives of isoxazole may be considered as “isoxazole-like compound” or “similar compound”. In an embodiment, the isoxazole derivatives such as 5-Amino-3-methyl-4-isoxazolecarboxylic acid semicarbazides and thiosemicarbazides may be synthesized. The reaction of 5-amino-3-methyl-4-isoxazolecarboxylic acid hydrazide with isocyanates and isothiocyanates may be designed and conducted. The isocyanates, in the reaction of nucleophilic addition with compounds containing the primary amino group, form urea derivatives and isothiocyanates the thiourea derivatives. Only the hydrazide terminal group (—NH2) participates in this reaction. The amino group in position 5 of isoxazole ring remains not reactive under the reaction conditions. The mechanism of the reaction consists in nucleophilic attack of the nitrogen atom in the hydrazide group (—NH2) on the carbon atom of isocyanate or isothiocyanate. The intermediate forms appear which undergo amidoiminole tautomerization leading to formation of substituted 5-amino-3-methyl-4-isoxazolecarboxylic acid semicarbazides and thiosemicarbazides. In an embodiment, examples of isoxazole derivatives may comprise 5-sulfanilamido-isoxazoles of the general formula wherein R and R are lower alkyl and/or lower alkoxy alkyl groups. Sulfanilamide derivatives with the isoxazole ring attached in N-position of the sulfanilamide molecule may be generated. For example, sulfanilamide radical in 4-position of the isoxazole ring. Further, a sulfanilyl derivative of 5-amino-isoxazole namely, 5-sulfanilamido-3-methyl-isoxazole may also be considered as an isoxazole derivative. In an embodiment, both the 3- and 4-positions of the isoxazole ring of the sulfanilamide derivatives may be replaced by an alkyl and/or corresponding alkoxy alkyl radical to generate. Non-limiting examples of “isoxazole-like compound” or “similar compound” comprise 1,2-oxazole, 4-deuterio-1,2-oxazole, 1,2-oxazole;potassium, hydron;1,2-oxazole, 1-oxido-1,2-oxazol-1-ium, 1,2-oxazole;hydrobromide, 1,2-oxazole;hydrochloride, ethane;1,2-oxazole, potassium;1,2-oxazole;hydroxide, 1,2-oxazole;hydrate;hydrochloride, ethane;1,2-oxazole, 1,2-oxazole;cyanate, 2-oxido-1,2-oxazol-2-ium, carbon monoxide;chromium;1,2-oxazole, ethane;1,2-oxazole, ethane;1,2-oxazole;propane, 1,2-oxazol-2-ium-2-sulfonate, carbonyl dichloride;1,2-oxazole, isocyanic acid;1,2-oxazole, ethoxyethane;1,2-oxazole, 2,2-dimethylpropane;ethane;1,2-oxazole, ethane;methoxyethane;1,2-oxazole, ethane;2-methylpropane;1,2-oxazole, 1,2-oxazole;urea, ethanol;1,2-oxazole, carbonic acid;1,2-oxazole, 1,2-oxazol-1-ium-1-sulfonic acid, 1,2-oxazol-2-ium;iodide.

“Isoxazole 9” (ISX-9) is a small molecule inducer of adult neural stem cell differentiation both in vitro and in vivo (Schneider et al.). It has been shown to act through a calcium-activated signaling pathway dependent on myocyte-enhancer factor 2 (MEF2)-dependent gene expression (Schneider et al.; Petrik et al.). Compounds are also available from Sigma-Aldrich and StemCell Technologies. The molecular formula is C₁₁H₁₀N₂O₂S, and the chemical name is N-cyclopropyl-5-thiophen-2-yl-1,2-oxazole-3-carboxamide. The two-dimensional structure is:

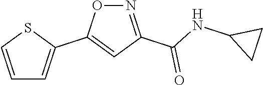 [SMILES: O=C(NC1CC1)c1cc(-c2cccs2)on1]

“Isoxazole 1” (ISX-1) is a small molecule having the structure:

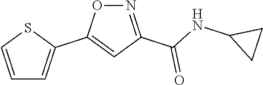 [SMILES: O=C(NC1CC1)c1cc(-c2cccs2)on1]

As used herein, the term “isoxazole-like compound” or “similar compound” intends an agent or small molecule that has the same functional property of the isoxazole as disclosed herein. Non-limiting examples include Cardionogen; CDNG1/vuc230, CDNG2/vuc198, and CDNG3/vuc247 (see Terri et al. (2011) Chem Biol., December 23 18(12):1658-1668). Non-limiting examples further include sulfisoxazole as described herein below. Yet a further example is leflunomide (Arava), also known as 5-methyl-N-[4-(trifluoromethyl)phenyl]-1,2-oxazole-4-carboxamide.

An isoxazole compound or derivative thereof can also be a compound of the formula:

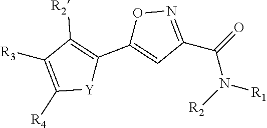

wherein R₁ and R₂ are both hydrogen or R₁ is hydrogen and R₂ is selected from the group consisting of substituted or unsubstituted C₁-C₆ alkyl, C₃-C₆ cycloalkyl, C₂-C₆ alkenyl, C₂-C₆ alkynyl, and benzyl, or where R₁ and R₂ may be joined together to form a ring selected from azetidinyl, pyrrolidinyl, piperidinyl, piperazinyl, or morpholinyl; R_{2′}, R₃ and R₄ are independently selected from the group consisting of hydrogen, halogen, C₁-C₆ alkyl, C₃-C₆ cycloalkyl, substituted or unsubstituted aromatic or heteroaromatic ring, cyano, nitro, and acyl; and Y is 0, NH or S.

In one aspect, an isoxazole compound has the formula:

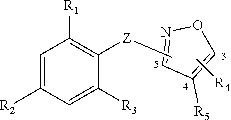

wherein R1 and R2 is each selected from C1-C4 alkyl, phenyl, benzyl, trifluoromethyl or halogen, R3 is selected from hydrogen, hydroxy, C1-C4 alkyl or alkoxy, R4, in position 3 or 5, is selected from hydrogen, trifluoromethyl, C1-C4 alkoxy, C1-C4 alkyl, or C1-C4 hydroxyalkyl, R5 is selected from hydrogen or C4-C4 alkyl or R4 and R5 together form a tetramethylene group, Z at position 3 or 5 on the heterocycle is selected from: —N(R6)-CO—, —CO—N(R6)-, —N(R6)-CO—N(R6)-, —CH(R6)-NH—CO—, or —NH—CO—CH(R6), in which R6 is selected from hydrogen or C1-C4 alkyl. Non-limiting examples include 5-(trifluoromethyl)-3-(4-methoxyphenyl)isoxazole-4-carboxylic acid, 5-(trifluoromethyl)-3-(4-fluorophenyl)isoxazole-4-carboxylic acid, 5-(thiophen-2-yl)isoxazole-3-carboxaldehyde, 5,6,7,8-tetrahydro-4h-cyclohepta[d]isoxazole-3-carboxylic acid, 4,5,6,7-tetrahydro-benzo[d]isoxazole-3-carboxylic acid, 3-amino-5-methylisoxazole, 4-amino-n-(5-methyl-3-isoxazolyl)benzenesulfonamide, 3-phenyl-isoxazole-5-boronic acid pinacol ester, 5-phenylisoxazole, 1-phenyl-1-cyclopentanecarboxylic acid, 3-phenyl-benzo[c]isoxazole-5-carboxylic acid, 5-methyl-3-phenylisoxazole-4-carboxylic acid, 3a,4,5,6,7,8,9,9a-octahydro-cycloocta[d]isoxazole-3-carboxylic acid, 5-(3-nitrophenyl)isoxazole, 3-(4-nitrophenyl)isoxazole, 3-hydroxy-5-aminomethyl-isoxazole, 5-(morpholinomethyl)isoxazole-3-carboxylic acid hydrochloride, 5-(morpholinomethyl)isoxazole-3-carbaldehyde, 3-methyl-5-(trifluoromethyl)isoxazole-4-carboxylic acid, methyl 5-(thiophen-2-yl)isoxazole-3-carboxylate, 3-(methylsulfonyl)-5-(2-thienyl)isoxazole-4-carbonitrile, 5-methyl-3-(2-pyrrolidinyl)isoxazole, 3-methyl-5-(2-pyrrolidinyl)isoxazole, 3-(1-methyl-1h-pyrazol-4-yl)-isoxazole-5-carboxylic acid, 3-(1-methyl-1h-pyrazol-4-yl)-4,5-dihydro-isoxazole-5-carboxylic acid, 5-(4-methylphenyl)isoxazole-3-carboxylic acid, 5-methyl-3-phenylisoxazole-4-carboxylic acid, 5-(4-methylphenyl)isoxazole-3-carboxaldehyde, 5-methyl-3-(4-phenoxyphenyl)isoxazole-4-carboxylic acid, 3-methyl-5-(4-methyl-1,2,3-thiadiazol-5-yl)isoxazole-4-carboxylic acid, 3-methyl-5-(5-methylisoxazol-3-yl)isoxazole-4-carboxylic acid, methyl 5-(4-methoxyphenyl)isoxazole-4-carboxylate, methyl 5-(4-methoxyphenyl)isoxazole-3-carboxylate, 5-methylisoxazole, methyl 5-(4-fluorophenyl)isoxazole-4-carboxylate, methyl 5-(4-fluorophenyl)isoxazole-3-carboxylate, methyl 5-(4-chlorophenyl)isoxazole-4-carboxylate, methyl 5-(4-bromophenyl)isoxazole-4-carboxylate, 5-(4-methoxyphenyl)isoxazole-3-carboxylic acid, 5-(3-methoxy-phenyl)-isoxazole-3-carboxylic acid, 3-(2-methoxyphenyl)isoxazole-5-carboxylic acid, 5-(4-methoxyphenyl)isoxazole-3-carboxaldehyde, 3-(4-methoxyphenyl)isoxazole-5-carbaldehyde, 3-(2-methoxyphenyl)isoxazole-5-carbaldehyde, 5-(4-methoxyphenyl)isoxazole, 3-(4-methoxyphenyl)isoxazole, 3-(2-methoxy-phenyl)-4,5-dihydro-isoxazole-5-carboxylic acid, 3-methoxy-isoxazole-5-carboxylic acid, isoxazole-5-carboxylic acid, isoxazole-4-carboxylic acid, isoxazole-5-carbothioamide, isoxazole-5-carbonyl chloride, isoxazole-3-carbonitrile, isoxazole-3-carbaldehyde, isoxazole-4-boronic acid, isoxazole, 5-cyclopropyl-4-[2-(methylsulfonyl)-4-(trifluoromethyl)benzoyl]isoxazole, 6-(5-(thiophen-2-yl)isoxazole-3-carboxamido)hexyl 5-((3as,4s,6ar)-2-oxohexahydro-1h-thieno[3,4-d]imidazol-4-yl)pentanoate, isocarboxazid 5-methyl-3-isoxazole-carboxylic acid 2-benzylhydrazide, 5-isobutyl-isoxazole-3-carboxylic acid, 4-iodo-5-methyl-isoxazole, 3,3′-iminobis(n,n-dimethylpropylamine), 3-(3-hydroxy-phenyl)-isoxazole-5-carboxylic acid methyl ester, 5-(4-hydroxy-phenyl)-isoxazole-3-carboxylic acid, 5-(3-hydroxy-phenyl)-isoxazole-3-carboxylic acid, 5-(hydroxymethyl)-3-methylisoxazole, 3-hydroxy-5-methylisoxazole, 5-(1-hydroxyethyl)-3-(4-trifluoromethylphenyl)isoxazole, 3a,4,5,6,7,7a-hexahydro-benzo[d]isoxazole-3-carboxylic acid, 5-(2-furyl)isoxazole-3-carbaldehyde, 5-furan-2-yl-isoxazole-3-carboxylic acid, 6-fluoro-3-(4-piperidinyl)benzisoxazole, 5-(4-fluorophenyl)isoxazole-3-methanol, 3-(2-fluoro-phenyl)-isoxazole-5-carboxylic acid, 5-(4-fluorophenyl)isoxazole-3-carboxaldehyde, 3-(4-fluorophenyl)isoxazole-5-carbaldehyde, 3-(3-fluorophenyl)isoxazole-5-carbaldehyde, 3-(2-fluorophenyl)isoxazole-5-carbaldehyde, 5-(4-fluorophenyl)isoxazole, 3-(4-fluorophenyl)isoxazole, 5-(3-fluoro-4-methoxy-phenyl)-isoxazole-3-carboxylic acid, ethyl 5-(trifluoromethyl)-3-(4-methoxyphenyl)isoxazole-4-carboxylate, ethyl-5-(tributylstannyl)isoxazole-3-carboxylate, ethyl 5-(thiophen-2-yl)isoxazole-3-carboxylate, 5-ethyl-isoxazole-4-carboxylic acid, 5-ethyl-isoxazole-3-carboxylic acid, ethyl 5-(4-fluorophenyl)isoxazole-4-carboxylate, ethyl 5-(4-fluorophenyl)isoxazole-3-carboxylate, ethyl 5-(2,3-dihydrobenzo[b][1,4]dioxin-7-yl)isoxazole-3-carboxylate, ethyl 3-(4-chlorophenyl)-5-(trifluoromethyl)isoxazole-4-carboxylate, ethyl 5-(4-chlorophenyl)isoxazole-3-carboxylate, ethyl 3-(4-bromophenyl)-5-(trifluoromethyl)isoxazole-4-carboxylate, ethyl 5-(4-bromophenyl)isoxazole-3-carboxylate, ethyl 5-amino-4-(4-chlorophenyl)isoxazole-3-carboxylate, ethyl 5-amino-4-(4-bromophenyl)isoxazole-3-carboxylate, ethyl 6b-acetyl-2-(acetyloxy)-4a,6a-dimethyl-2,3,4,4a,4b,5,6,6a,6b,9a,10,10a,10b,11-tetradecahydro-1h-naphtho[2′,1′:4,5]indeno[2,1-d]isoxazole-9-carboxylate, 3,5-dimethyl-4-(tributylstannyl)isoxazole, 5-(1,5-dimethyl-1h-pyrazol-4-yl)-isoxazole-3-carboxylic acid, 5-(1,3-dimethyl-1h-pyrazol-4-yl)-isoxazole-3-carboxylic acid, 5-(1,5-dimethyl-1h-pyrazol-4-yl)-isoxazole, 3,5-dimethylisoxazole-4-boronic acid pinacol ester, 3,5-dimethylisoxazole, 3-(dimethylamino)-1-(2-pyridyl)-2-propen-1-one, 5-(3,5-difluorophenyl)isoxazole, [2,6-dichloro-4-(trifluoromethyl)phenyl]hydrazine, 5-(2,5-dichlorophenyl)isoxazole-3-carboxylic acid, danazol, 3-(4-chlorophenyl)-5-(trifluoromethyl)isoxazole-4-carboxylic acid, 5-(4-chlorophenyl)isoxazole-3-propionic acid, 5-(4-chlorophenyl)isoxazole-4-carboxylic acid, 5-(4-chlorophenyl)isoxazole-3-carboxylic acid, 3-(4-chlorophenyl)isoxazole-5-carboxylic acid, 3-(3-chlorophenyl)isoxazole-5-carboxylic acid, 5-(4-chlorophenyl)isoxazole-3-carboxaldehyde, 3-(4-chlorophenyl)isoxazole-5-carbaldehyde, 3-(3-chlorophenyl)isoxazole-5-carbaldehyde, 3-(2-chlorophenyl)isoxazole-5-carbaldehyde, 5-(4-chlorophenyl)isoxazole, 3-(4-chlorophenyl)isoxazole, 5-(chloromethyl)isoxazole-4-carboxylic acid, 3-(chloromethyl)-5-(2-furyl)isoxazole, 4-chloromethyl-3,5-dimethylisoxazole, 5-(chloromethyl)-3-(4-chlorophenyl)isoxazole, 5-(3-chloro-4-methoxy-phenyl)-isoxazole-3-carboxylic acid, 3-chloro-4-fluorobenzaldehyde, 3-(5-chloro-2,4-dimethoxy-phenyl)-4,5-dihydro-isoxazole-5-carboxylic acid, 5-tert-butyl-4,5,6,7-tetrahydro-benzo[d]isoxazole-3-carboxylic acid, 3-(4-bromophenyl)-5-(trifluoromethyl)isoxazole-4-carboxylic acid, 5-(4-bromophenyl)isoxazole-3-propionic acid, 5-(4-bromophenyl)isoxazole-3-carboxylic acid hydrazide, 5-(4-bromophenyl)isoxazole-4-carboxylic acid, 5-(4-bromophenyl)isoxazole-3-carboxylic acid, 3-(4-bromophenyl)isoxazole-5-carboxylic acid, 3-(4-bromophenyl)isoxazole-5-carboxaldehyde, 5-(4-bromophenyl)isoxazole, 5-(3-bromophenyl)isoxazole, 3-(4-bromophenyl)isoxazole, 5-(bromomethyl)-3-(4-methoxyphenyl)isoxazole, 4-(bromomethyl)isoxazole, 5-(bromomethyl)-3-(4-fluorophenyl)isoxazole, 5-(bromomethyl)-3-(4-chlorophenyl)isoxazole, 5-(bromomethyl)-3-(4-bromophenyl)isoxazole, 6-bromo-3-methylbenzo[d]isoxazole, 5-bromo-3-methylbenzo[d]isoxazole, 4-bromo-5-(4-methoxyphenyl)isoxazole, 3-bromo-isoxazole, 3-bromo-5-(2-hydroxyethyl)isoxazole, 4-bromo-5-(4-fluorophenyl)isoxazole, 3-bromo-5-(4-fluorophenyl)isoxazole, 4-bromo-5-(4-chlorophenyl)isoxazole, 4-bromo-5-(4-bromophenyl)isoxazole, 6-bromo-benzo[d]isoxazole-3-carboxylic acid, benzo[d]isoxazole-3-carboxylic acid, 3-amino-5-methylisoxazole, 5-amino-3-(4-methoxyphenyl)isoxazole, 3-aminoisoxazole, 3-amino-5-(4-fluorophenyl)isoxazole, 5-amino-3-(4-chlorophenyl)isoxazole, 5-amino-4-(4-bromophenyl)isoxazole, 3-amino-5-(4-bromophenyl)isoxazole, 5-acetyl-3-(4-fluorophenyl)isoxazole, 5-acetyl-3-(3-fluorophenyl)isoxazole, 3-methyl-5-[(2s)-1-methyl-2-pyrrolidinyl]isoxazole hydrochloride, 7-methoxy-5-methyl-4,5-dihydronaphtho[2,1-d]isoxazole, 5-methyl-3-phenyl-isoxazole-4-carboxylic acid methylamide, 5-methyl-3-phenyl-isoxazole-4-carbothioic acid methylamide, 5-methyl-3-phenyl-4-(1h-pyrazol-5-yl)isoxazole, 5-benzyl-3-furan-2-yl-2-phenyl-tetrahydro-pyrrolo(3,4-d)isoxazole-4,6-dione, 5-benzyl-3-[4-(dimethylamino)phenyl]-2-phenyldihydro-2h-pyrrolo[3,4-d]isoxazole-4,6(3h,5h)-dione, 5-benzyl-3-(5-br-2-ho-phenyl)-2-ph-tetrahydro-pyrrolo(3,4-d)isoxazole-4,6-dione, 5-benzyl-3-(4-nitro-ph)-2-ph-dihydro-2h-pyrrolo(3,4-d)isoxazole-4,6(3h,5h)-dione, 5-benzyl-3-(4-methoxy-phenyl)-2-ph-tetrahydro-pyrrolo(3,4-d)isoxazole-4,6-dione, 5-benzyl-3-(4-fluorophenyl)-2-(2-methylphenyl)dihydro-2h-pyrrolo[3,4-d]isoxazole-4,6(3h,5h)-dione, 5-benzyl-3-(3-nitro-ph)-2-phenyl-tetrahydro-pyrrolo(3,4-d)isoxazole-4,6-dione, 5-benzyl-2-ph-3-(2-pyridinyl)dihydro-2h-pyrrolo(3,4-d)isoxazole-4,6(3h,5h)-dione, 5-benzyl-2-ph-3-(2-ph-vinyl)dihydro-2h-pyrrolo(3,4-d)isoxazole-4,6(3h,5h)-dione, 5-benzyl-2-(4-chlorophenyl)-3-(2-thienyl)dihydro-2h-pyrrolo[3,4-d]isoxazole-4,6(3h,5h)-dione, 5-benzyl-2,3-diphenyldihydro-2h-pyrrolo(3,4-d)isoxazole-4,6(3h,5h)-dione, 5-benzyl-2(4-cl-ph)3-(2-furyl)dihydro-2h-pyrrolo(3,4-d)isoxazole-4,6(3h,5h)dione, 5-benzyl-2(4-cl-ph)-3-(4-f-ph)dihydro-2h-pyrrolo(3,4-d)isoxazole-4,6(3h,5h)dione, 5-(p-tolyl)isoxazole, 5-(4-methylphenyl)-3-(4-nitrophenyl)-2-phenyldihydro-2h-pyrrolo[3,4-d]isoxazole-4,6(3h,5h)-dione, 5-(4-methoxyphenyl)-2-phenyl-3-(4-pyridinyl)dihydro-2h-pyrrolo[3,4-d]isoxazole-4,6(3h,5h)-dione, 5-(4-methoxyphenyl)-2-phenyl-3-(3-pyridinyl)dihydro-2h-pyrrolo[3,4-d]isoxazole-4,6(3h,5h)-dione, 5-(4-methoxy-ph)-2,3-diphenyldihydro-2h-pyrrolo(3,4-d)isoxazole-4,6(3h,5h)-dione, 5-(4-fluorophenyl)-3-(4-methoxyphenyl)-2-phenyldihydro-2h-pyrrolo[3,4-d]isoxazole-4,6(3h,5h)-dione, 5-(4-fluorophenyl)-2-(2-methylphenyl)-3-(4-pyridinyl)dihydro-2h-pyrrolo[3,4-d]isoxazole-4,6(3h,5h)-dione, 5-(4-ethoxyphenyl)-3-(4-nitrophenyl)-2-phenyldihydro-2h-pyrrolo[3,4-d]isoxazole-4,6(3h,5h)-dione, 5-(4-ethoxyphenyl)-3-(4-fluorophenyl)-2-phenyldihydro-2h-pyrrolo[3,4-d]isoxazole-4,6(3h,5h)-dione, 5-(4-ethoxyphenyl)-2-methyl-3-(4-nitrophenyl)dihydro-2h-pyrrolo[3,4-d]isoxazole-4,6(3h,5h)-dione, 5-(4-ethoxy-ph)-2-ph-3-thiophen-2-yl-4h-pyrrolo(3,4-d)isoxazole-4,6-dione, 5-(4-cl-ph)-3-(3-nitro-ph)-2-phenyl-tetrahydro-pyrrolo(3,4-d)isoxazole-4,6-dione, 5-(4-bromophenyl)-3-(4-methoxyphenyl)-2-phenyldihydro-2h-pyrrolo[3,4-d]isoxazole-4,6(3h,5h)-dione, 5-(4-bromophenyl)-3-(2-furyl)-2-(2-methylphenyl)dihydro-2h-pyrrolo[3,4-d]isoxazole-4,6(3h,5h)-dione, 5-(4-bromophenyl)-2-phenyl-3-(2-pyridinyl)dihydro-2h-pyrrolo[3,4-d]isoxazole-4,6(3h,5h)-dione, 5-(4-br-ph)2-ph-3-(2-ph-vinyl)dihydro-2h-pyrrolo(3,4-d)isoxazole-4,6(3h,5h)dione, 5-(2-cl-ph)-3-(4-dimethylamino-ph)2-o-tolyl-4h-pyrrolo(3,4-d)isoxazole-4,6-dione, 5-(2-chlorophenyl)-3-[4-(dimethylamino)phenyl]-2-phenyldihydro-2h-pyrrolo[3,4-d]isoxazole-4,6(3h,5h)-dione, 5-(2-chlorophenyl)-3-(4-methoxyphenyl)-2-phenyldihydro-2h-pyrrolo[3,4-d]isoxazole-4,6(3h,5h)-dione, 4-((3-(2-cl-ph)-5-methyl-isoxazole-4-carbonyl)-amino)-benzoic acid ethyl ester, 4,5,6,6a-tetrahydro-3ah-cyclopenta[d]isoxazole-3-carboxylic acid, 3-phenyl-3a,6a-dihydrothieno[2,3-d]isoxazole 4,4-dioxide, 3-methyl-5-(3-phenylpropyl)isoxazole, 3-methyl-4-nitro-5-[(e)-2-phenylethenyl]isoxazole, 3-methyl-4,5,8,9-tetrahydrocycloocta(d)isoxazole, 3-methyl-4,5,5a,6a,7,8-hexahydrooxireno(2′,3′:5,6)cycloocta(1,2-d)isoxazole, 3-methyl-3a,4,5,8,9,9a-hexahydrocycloocta(d)isoxazole, 3-furan-2-yl-2-phenyl-5-p-tolyl-tetrahydro-pyrrolo(3,4-d)isoxazole-4,6-dione, 3-chloro-4,5-dihydro(1)-benzothiepino(5,4-c)isoxazole, 3-[4-(dimethylamino)phenyl]-5-(4-methoxyphenyl)-2-(2-methylphenyl)dihydro-2h-pyrrolo[3,4-d]isoxazole-4,6(3h,5h)-dione, 3-(5-br-2-ho-phenyl)-2,5-diphenyl-tetrahydro-pyrrolo(3,4-d)isoxazole-4,6-dione, 3-(5-br-2-ho-ph)-5-(2-cl-ph)-2-ph-tetrahydro-pyrrolo(3,4-d)isoxazole-4,6-dione, 3-(4-meo-phenyl)-5-phenyl-2-o-tolyl-tetrahydro-pyrrolo(3,4-d)isoxazole-4,6-dione, 3-(4-fluorophenyl)-5-(4-nitrophenyl)-2-phenyldihydro-2h-pyrrolo[3,4-d]isoxazole-4,6(3h,5h)-dione, 3-(4-fluorophenyl)-5-(4-methylphenyl)-2-phenyldihydro-2h-pyrrolo[3,4-d]isoxazole-4,6(3h,5h)-dione, 3-(4-dimethylamino-ph)-5-ph-2-o-tolyl-4h-pyrrolo(3,4-d)isoxazole-4,6-dione, 3-(4-fluorophenyl)-5-(4-methoxyphenyl)-2-phenyldihydro-2h-pyrrolo[3,4-d]isoxazole-4,6(3h,5h)-dione, 3-(4-br-ph)-2-ph-5-(2-trifluoromethyl-ph)-4h-pyrrolo(3,4-d)isoxazole-4,6-dione, 3-(3-nitro-phenyl)-2,5-diphenyl-tetrahydro-pyrrolo(3,4-d)isoxazole-4,6-dione, 3-(3-br-phenyl)-2,5-diphenyldihydro-2h-pyrrolo(3,4-d)isoxazole-4,6(3h,5h)-dione, 3-(3-br-ph)-5-(2-meo-ph)-2-o-tolyl-tetrahydro-pyrrolo(3,4-d)isoxazole-4,6-dione, 3-(2-furyl)-5-[4-(4-morpholinyl)phenyl]-2-phenyldihydro-2h-pyrrolo[3,4-d]isoxazole-4,6(3h,5h)-dione, 3-(2-furyl)-2-(2-me-ph)-5-ph-dihydro-2h-pyrrolo(3,4-d)isoxazole-4,6(3h,5h)-dione, 3-(2-furyl)-2,5-diphenyldihydro-2h-pyrrolo(3,4-d)isoxazole-4,6(3h,5h)-dione, 3-(2-cl-phenyl)-5-methyl-isoxazole-4-carboxylic acid (2,5-dichloro-phenyl)-amide, 3-(2-cl-ph)-5-me-isoxazole-4-carboxylic acid (4,5-dihydro-thiazol-2-yl)-amide, 3-(2-chloro-phenyl)-5-methyl-isoxazole-4-carboxylic acid cyanomethyl-amide, 3-(2,4-dichlorophenyl)-5-(4-methoxyphenyl)-2-phenyldihydro-2h-pyrrolo[3,4-d]isoxazole-4,6(3h,5h)-dione, 3-(2,4-di-cl-ph)-2,5-diphenyldihydro-2h-pyrrolo(3,4-d)isoxazole-4,6(3h,5h)-dione, 3-(2,2-dichloro-vinyl)-5-phenyl-isoxazole, 3,5-diphenyl-isoxazole, 3,5-dimethyl-4-(1-pyrrolidinylsulfonyl)isoxazole, 3(4-dimethylamino-ph)-5-(4-eto-ph)2-o-tolyl-4h-pyrrolo(3,4-d)isoxazole-4,6-dione, 2-(4-cl-ph)5-ph-3-(2-thienyl)dihydro-2h-pyrrolo(3,4-d)isoxazole-4,6(3h,5h)-dione, 2-(4-cl-ph)-5-(3-meo-ph)-3-(3-nitro-ph)-4h-pyrrolo(3,4-d)isoxazole-4,6-dione, 2-(4-cl-ph)-3-(4-meo-ph)-5-p-tolyl-tetrahydro-pyrrolo(3,4-d)isoxazole-4,6-dione, 2-(4-chlorophenyl)-5-(4-methylphenyl)-3-(2-thienyl)dihydro-2h-pyrrolo[3,4-d]isoxazole-4,6(3h,5h)-dione, 2-(4-chlorophenyl)-3-[4-(dimethylamino)phenyl]-5-phenyldihydro-2h-pyrrolo[3,4-d]isoxazole-4,6(3h,5h)-dione, 2-(4-chlorophenyl)-3-(4-fluorophenyl)-5-(4-nitrophenyl)dihydro-2h-pyrrolo[3,4-d]isoxazole-4,6(3h,5h)-dione, 2-(4-chlorophenyl)-3-(2-thienyl)-5-[3-(trifluoromethyl)phenyl]dihydro-2h-pyrrolo[3,4-d]isoxazole-4,6(3h,5h)-dione, 2-(4-chlorophenyl)-3-(2,4-dichlorophenyl)-5-phenyldihydro-2h-pyrrolo[3,4-d]isoxazole-4,6(3h,5h)-dione, 2,3-di-ph-5-(3-(tri-f-me)ph)dihydro-2h-pyrrolo(3,4-d)isoxazole-4,6(3h,5h)-dione, danazol, and n-cyclopropyl-5-(thiophen-2-yl)isoxazole-3-carboxamide. In a further aspect, the isoxazole compound is danazol or n-cyclopropyl-5-(thiophen-2-yl)isoxazole-3-carboxamide. Additional non-limiting examples include compounds of the isoxazole compound has the general formula (I):

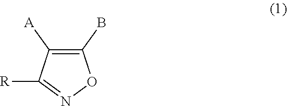

wherein A is a heterocyclic group optionally substituted with G or phenyl optionally substituted with G, wherein G is halogen, C1-C6 alkyl, or optionally substituted phenyl; B is phenyl substituted with halogen, or a heterocyclic group substituted with halogen, C1-C6 alkyl; and R is hydrogen, or C1-C6 alkyl.

Givinostat (GIV) is an orally bioavailable hydroxymate inhibitor of histone deacetylase (HDAC) with potential anti-inflammatory, anti-angiogenic, and antineoplastic activities. Givinostat inhibits class I and class II HDACs, resulting in an accumulation of highly acetylated histones, followed by the induction of chromatin remodeling and an altered pattern of gene expression. At low, nonapoptotic concentrations, this agent inhibits the production of pro-inflammatory cytokines such as tumor necrosis factor- (TNF-), interleukin-1 (IL-1), IL-6 and interferon-gamma. Givinostat has also been shown to activate the intrinsic apoptotic pathway, inducing apoptosis in hepatoma cells and leukemic cells. This agent may also exhibit anti-angiogenic activity, inhibiting the production of angiogenic factors such as IL-6 and vascular endothelial cell growth factor (VEGF) by bone marrow stromal cells. Previously publications by us describe in detail the methods and results of using GIV to produce IPS derived muscle progenitor cells which also improved dystrophin in mice with duchenne muscular dystrophy[99]. CHIR99021 is an aminopyrimidine derivative that is an extremely potent inhibitor of GSK3, inhibiting GSK30 (ICII=6.7 nM) and GSK3a (ICII=10 nM) and functions as a WNT activator. It is the most selective inhibitor of GSK3 reported so far. CHIR99021 is available from Sigma-Aldrich and StemCell Technologies.

Rho-associated kinase (ROCK) inhibitors intend Rho-associated protein kinase (ROCK) is a kinase belonging to the AGC (PKA/PKG/PKC) family of serine-threonine kinases. It is involved mainly in regulating the shape and movement of cells by acting on the cytoskeleton. Non-limiting examples of such include Thiazovivin or Y27632, which both can be purchased from Stemcell Technologies and respectively; SR3677, which can be purchased from tocris.com; and GSK429286, which can be purchased from tocris.com.

A TGF-beta type-I receptor inhibitor intends (activin A receptor type II-like kinase, 53 kDa) is an inhibitor for membrane-bound receptor protein for the TGF beta superfamily of signaling ligands. TGFBR1 is its human gene. Non-limiting examples of such include SB431542 and A8301 that can be purchased from _www.tocris.com_ and _www.esibio.com_, respectively; LY2157299, which can be purchased from www.selleckchem.com; and LY2109761, which can be purchased from www.selleckchem.com.

A DNA methyltransferase inhibitor is a small molecule or other agent the ability to inhibit hypermethylation, restore suppressor gene expression and exert antitumor effects in in vitro and in vivo laboratory models. Goffin and Eisenhauer (2002) Ann. Oncol. November 13(11):1699-16716. One non-limiting example of such an inhibitor is N-phthalyl-L-tryptopha (C₁₉H₁₄N₂O₄, sold under the tradename RG108, Sigma-Aldrich). Additional examples include 5′-azacytidine, 5-azacytidine, antisense oligonucleotides to methyltransferase 1, e.g., MG98 (see Amato (2007) Clin. Gentourin Cancer, December, 5(7):422-426 and 1-(β-D-Ribofuranosyl)-2(1H)-pyrimidinone (a nucleoside analog of cytidine, sold under the name Zebularine (Abcam®).

DNA hypomethylation intends a lower than normal level of DNA methylation. Methods of determining the level of DNA methylation are known in the art, some of which are described herein.

“Sulfisoxazole” is a sulfonamide antibacterial with an oxazole substituent. It has antibiotic activity against a wide range of Gram-negative and Gram-positive organisms. Compounds of this class available from Sigma-Aldrich and methods to synthesize such are known in the art as described for example in U.S. Pat. No. 2,721,200. Non-limiting examples of sulfisoxazole include FDA approved drugs of AZO GANTRISIN, ERYTHROMYCIN ETHYLSUCCINATE and SULFISOXAZOLE ACETYL, ERYZOLE, GANTRISIN (with effective ingredients as SULFISOXAZOLE), GANTRISIN (with effective ingredients as SULFISOXAZOLE ACETYL), GANTRISIN (with effective ingredients as SULFISOXAZOLE ACETYL), GANTRISIN PEDIATRIC, ILOSONE SULFA, LIPO GANTRISIN, PEDIAZOLE, SOSOL, SOXAZOLE, SOXAZOLE, SULFISOXAZOLE, SULFISOXAZOLE DIOLAMINE, and SULSOXIN. (Drugs@FDA: FDA Approved Drug Products at the website of accessdata.fda.org).

Danazol (also known as 17a-Ethynyl-170-hydroxyandrost-4-en-[2,3-d]isoxazole) is a synthetic steroid that is used primarily in the treatment of endometriosis. The compound is commercially available and manufactured by a variety of vendors.

The term “isolated” as used herein refers to molecules or biological or cellular materials being substantially free from other materials, e.g., greater than 70%, or 80%, or 85%, or 90%, or 95%, or 98%. In one aspect, the term “isolated” refers to nucleic acid, such as DNA, RNA, miRNA, exosome or microvesicle, protein or polypeptide, or cell or cellular organelle, or tissue or organ, separated from other DNA, RNA, miRNA, exosome or microvesicle, protein or polypeptide, or cell or cellular organelle, or tissue or organ, respectively, that are present in the natural source and which allow the manipulation of the material to achieve results not achievable where present in its native or natural state, e.g., recombinant replication or manipulation by mutation. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments that are not naturally occurring as fragments and would not be found in the natural state. The term “isolated” is also used herein to refer to polypeptides which are isolated from other cellular proteins and is meant to encompass both purified and recombinant polypeptides, e.g., with a purity greater than 70%, or 80%, or 85%, or 90%, or 95%, or 98%. The term “isolated” is also used herein to refer to cells, exosomes or microvesicles, miRNA, or tissues that are isolated from other cells, exosomes or microvesicles, miRNA, or tissues and is meant to encompass both cultured and engineered cells or tissues and products produced or isolated from such.

The term “phenotype” refers to a description of an individual's trait or characteristic that is measurable and that is expressed only in a subset of individuals within a population. In one aspect of the invention, an individual's phenotype includes the phenotype of a single cell, a substantially homogeneous population of cells, a population of differentiated cells, or a tissue comprised of a population of cells.

The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present invention include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

A population of cells intends a collection of more than one cell, exosome or microvesicle, or miRNA that is identical (clonal) or non-identical in phenotype and/or genotype. The population can be purified, highly purified, substantially homogenous or heterogeneous as described herein.

The term “propagate” means to grow or alter the phenotype of a cell or population of cells. The term “growing” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue. In yet another embodiment, the tissue is comprised of cardiac progenitor cells or cardiac cells.

The term “effective amount” refers to a concentration or amount of a reagent or composition, such as a composition as described herein, cell population or other agent, that is effective for producing an intended result, including cell growth and/or differentiation in vitro or in vivo, or for the treatment of a condition as described herein. It will be appreciated that the number of cells to be administered will vary depending on the specifics of the disorder to be treated, including but not limited to size or total volume/surface area to be treated, as well as proximity of the site of administration to the location of the region to be treated, among other factors familiar to the medicinal biologist.

The terms effective period (or time) and effective conditions refer to a period of time or other controllable conditions (e.g., temperature, humidity for in vitro methods), necessary or preferred for an agent or composition to achieve its intended result, e.g., the differentiation or dedifferentiation of cells to a pre-determined cell type.

A “subject,” “individual” or “patient” is used interchangeably herein, and refers to a vertebrate, preferably a mammal, more preferably a human. Mammals include, but are not limited to, murines, rats, simians, bovines, canines, felines, humans, farm animals, sport animals and pets.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker.

As used herein, the terms “treating,” “treatment” and the like are used herein to mean obtaining a desired pharmacologic and/or physiologic effect. The effect can be prophylactic in terms of completely or partially preventing a disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder, e.g., cardiac arrhythmia. As is understood by those skilled in the art, “treatment” can include systemic amelioration of the symptoms associated with the pathology and/or a delay in onset of symptoms such as chest pain. Clinical and sub-clinical evidence of “treatment” will vary with the pathology, the individual and the treatment.

“Administration” or “delivery” of a cell, exosome or microvesicle, miRNA, therapeutic or other agent and compositions containing same can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application.

### BACKGROUND OF THE DISCLOSURE

Ischemic heart disease is the major cause of death and morbidity in the developed countries. Cardiac progenitor cells (CPCs) are recognized as potential candidates for cell-based therapy to treat myocardial infarction. These multipotent cells promote beneficial effects through angiomyogenesis, anti-inflammatory, pro-survival and anti-fibrotic mechanisms. Indeed, the inventor has an issued patent (U.S. Ser. No. 10/443,044) and a number of pending applications directed in part to making and using CPCs to treat cardiac disease (US20150297638, US2016076001, US2018273906, each incorporated by reference in its entirety for all purposes).

In one embodiment, the extracellular vesicle is an exosome, a nanovesicle, an apoptotic body, a microvesicle, a lysosome, an endosome, an enveloped virus, a viral vector, a liposome, a lipid nanoparticle, a micelle, a multilamellar structure, a revesiculated vesicle, an extruded cell or cytokine or chemokine. Exosomes are cell-derived vesicles that are present in many, and perhaps all, eukaryotic fluids including blood, urine, cerebrospinal fluid and cultured medium of cell cultures. Exosomes are approximately 30-200 nm sized vesicular structures containing proteins, surface proteins and nucleic acids. Exosomes are potent carriers of extracellular RNA (exosomal shuttle RNA) and are shown to transfer functional microRNAs into recipient cells.

The beneficial effects by CPCs are believed to be reproduced by their extracellular vesicles & exosomes, which carry a distinctive load of bioactive molecules and miRNAs. These extracellular vesicles & exosomes promote angiogenesis, proliferation of myocytes and block formation of scar tissue in the infarcted heart. Efforts have shown that exosomes from CPC cells induced with ISX-9 exerted strong therapeutic effect on fibrosis and angiogenesis in mice with myocardial infarction (“MI”) (preparing for publication). Amongst highly enriched miRNAs, we have found that miRNA-373 was strongly antifibrotic targeting 2 genes, GDF-11 and ROCK-2[98]. The miRNA-373 mimic itself was highly efficacious in preventing scar formation in the infarcted myocardium with strong therapeutic implications. When discussion herein is about miRNA-373, such discussion is meant to include any of the known or to be discovered/invented mimics that have the same functionality[98]. Detailed methods and results in our previous publication describe our gene therapy approach for reversal of cardiac fibrosis and showed strong functional heart generation by using miRNA-373 mimic, exosomal miRNA-373 and also their targeting of, GDF-11 and ROCK-2 genes[98].

“MicroRNAs aka miRNAs” are short (20-24 nt) non-coding RNAs that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce an approximately 70-nt stem-loop precursor miRNA (pre-miRNA), which is further cleaved by the cytoplasmic Dicer ribonuclease to generate the mature miRNA and antisense miRNA star (miRNA*) products. The mature miRNA is incorporated into a RNA-induced silencing complex (RISC), which recognizes target mRNAs through imperfect base pairing with the miRNA and most commonly results in translational inhibition or destabilization of the target mRNA. The RefSeq represents the predicted microRNA stem-loop. [provided by RefSeq, September 2009]

MicroRNA373 is known by multiple names and in many databases. mir-373 is annotated as ENSG00000199143 in ENSEMBLE, in miRbase as MI0000781, NCBI provides Acc. No. 442918 (see ncbi.nlm.nih.gov/gene/442918), and EntrezGene and HGNC provide MIR373. Another type of mature miRNA, or miR-373, which can be used in the present invention has the common seed sequence gaagugcu on the 5′ side of the miRNA to be a member of a miR-373 family. The sequence of the miRNA in the miR-373 family is indicated below, with the seed sequence underlined: miR-373:

(SEQ ID NO. 3)
gaagugcuucgauuuuggggugu.

Another group of miRNAs according to the present disclosure may comprise mature miRNAs that contain the seed sequence of gaagugcu at the 5′ end and precursors of said miRNAs, as well as variants or analogs thereof. These miRNAs comprise miRNAs selected from the group consisting of miR-, precursors of said miRNA, as well as variants and analogs thereof. For example, precursors of the mature miRNA, i.e., miR-373, include pri-miRNAs and pre-miRNAs of this miRNA. Specific examples include the precursor represented are depicted below: human miR-373

(SEQ ID NO: 1 in table 1)
gggauacuca aaaugggggc gcuuuccuuu uugucuguac
ugggaagugc uucgauuuug ggguguccc.

Examples of miRNA 373 mimic's are known and sold by Sigma-Aldrich under MISSION® microRNA Mimic hsa-miR-373* (sigmaaldrich.com/catalog/product/sigma/hmi0531?lang=en&region=US). Additional Micro-ma 373 mimic's, mir-373 inhibitors, mir-373 oglio's, mir-373 expression vector's, mir-373 precursers are known and sold by: SwitchGear Genomics, Inc. (switchgeargenomics.com/products/lightswitch-mirna-mimics-inhibitors/inhibitors/mir-300-399 (mir373 inhibitors; switchgeargenomicscom/products/lightswitch-mirna-mimics-inhibitors/mir-300-399. (miR373 mimics (switchgeargenomics.com/products/synthetic-3utr-goclone-reporters/mir-300-399 (miR 3′UTR reporter)); (addgene.org/78127: SEQ ID NO:4 and SEQ ID NO:5, see Table below) (miR-373 expression vector); Thermo Fisher Scientific Inc. (thermnofisher.com/us/en/home/life-science/epigenetics-noncoding-mna-research/mirna-analysis/mimna-mimics-inhibitors/mirvana-mimics-inhibitors.html and thermofisher.com/us/en/home/life-science/epigenetics-noncoding-rna-research/mimna-analysis/mimna-mimics-inhibitors/ambion-pre-mir-precursors.html. Sequences are provided in Table 1:

| | | SEQ ID | |
|---|---|---|---|
| NAME | ACC. NO. or SOURCE | NO: | SEQUENCE |
| Hsa-mir-373 | MI0000781 | 1 | GGGAUACUCA |
| | | | AAAUGGGGGC |
| | | | GCUUUCCUUU |
| | | | UUGUCUGUAC |
| | | | UGGGAAGUGC |
| | | | UUCGAUUUUG |
| | | | GGGUGUCCC |
| hsa-nniR-373-5p | MIMAT0000725 | 2 | ACUCAAAAUG |
| | | | GGGGCGCUUU CC |
| hsa-miR-373-3p | MIMAT0000726 | 3 | GAAGUGCUUC |
| | | | GAUUUUGGGG UGU |
| miR373 in pcDNA3.1 | Plasmid #78127 from | 4 | (forward) CTCGAGATCT |
| | AddGene | | GGGGATACTC |
| | | | AAAATGGGGG |
| | | | CGCTTTCCTT |
| | | | TTTGTCTGTACTGG |
| | | 5 | (reverse) CTCGAGGATC |
| | | | CGGGACACCC |
| | | | CAAAATCGAA |
| | | | GCACTTCCCA |
| | | | GTACAGACAA AAA |
| Human | UniProtKB - O75116 | 6 | GCTGCAGTTG |
| ROCK2 | | | CAACTATGCA CTTG |
| ROCK2_Mouse | UniProtKB - P70336 | 7 | ATTTCAGTTG |
| | | | CAACTATGCA CTTG |
| ROCK2_Rat | UniProtKB - Q62868 | 8 | ATTTCAGTTG |
| | | | CAACTATGCA CTTG |
| ROCK2_Horse | UniProtKB - F6QSI7 | 9 | ACTGCAGTTG |
| | | | CAACTATGCA CTTG |
| ROCK2_Sheep | UniProtKB - W5Q2Y6 | 10 | ACTGCAGTTG |
| | | | CAACTATGCA CTTG |
| ROCK2_Chicken | UniProtKB - | 11 | ATTGCAGTTG |
| | A0A3Q2TTH6 | | CAACTATGCA CTTG |
| hsa-miR-373-3p | | 12 | TGTGGGGTTT |
| | | | TAGCTTCGTG AAG |
| miRNA-373 inhibitor | | 13 | ACACCCCAAA |
| | | | AUCGAAGCAC UUC |
| Inventor's plasmid | | 14 | GAATTCACTA |
| | | | GTACCGGTAG |
| | | | GCCTGTCGAC |
| | | | GATATCGGGC |
| | | | CCGCGGCCGC |
| | | | TGGATCCTCT |
| | | | AGACTCGAG |
| Hsa-miR-373 (ROCK2) | [Figure 24D] | 16 | UGUGGGGUUU |
| | | | UAGCUUCGUG AAG |
| ROCK2-3′UTR-Mut | [Figure 24D] | 17 | AATGGGAAAA |
| | | | CAACTATAAG GCC |
| Hsa-GDF113′UTR (704- | [Figure 24D] | 18 | ACACCUACUC |
| 710) | | | ACUUAAGCAC UUG |
| Hsa-miR-373 (GDF11) | [Figure 24D] | 19 | UGUGGGGUUU |
| | | | UAGCUUCGUG AAG |
| GDF-11-3′UTR-Mut | [Figure 24D] | 20 | TTTGGGACTC |
| | | | ACTTCCCGGA AA |

A human miR-373 mimetic compound comprises two nucleotide strands, each 22-26 bases, in which the first strand is identical the sequence of mature miR-371, miR-372, miR-373, and miR-373*, and the second strand is significantly complementary to the first strand and has least modified nucleotide, such that when the two strands bind one another the first strand has a 3′ nucleotide overhang relative to the second strand.

The prior application (CIP: 62/807,647) proposed to combine CPC'S with an excess of exosomes from CPCs, and the combination predicted to have synergistic effects over the use of either component alone. Presented herein is actual data confirming the original hypothesis. In addition, a related clinical proof of concept has recently occurred in humans with the Japan Times reporting Jan. 28, 2020 the first successful transplantation of IPS derived cardiomyocytes for the start of Dr. Yoshiki Sawa's clinical trial in Japan.

### SUMMARY OF THE INVENTION

Prepared herein are CPCs from human induced pluripotent stem cells (hiPS cells) with the treatment of a “special small molecule” possessing antioxidant, prosurvival and regenerative properties and which were designated as smart CPCs (see e.g. US20150297638, US2016076001 US2017002329, US2018273906, each incorporated herein by reference in its entirety for all purposes). It is proposed herein that exosomes from these smart CPCs will be superior in their regenerative and cadioprotective capacity compared to ordinary CPCs. Further posited is that combined administration of smart CPCs and an excess of their exosomes will accelerate a repair process through regeneration and paracrine factors.

These hypotheses have been tested in the following specific Aims. In our first specific aim, human iPSC reprogrammed CPCs with an isoxazole (e.g., ISX-9 [CAS 832115-62-5] or oxazole [CAS Number: 288-14-2]) possessing anti-oxidative, anti-inflammatory and cardiac gene promoting properties will maximize regenerative capacity of IPSC and their derivatives; We have developed a novel, cell free CPC based therapeutic approach utilizing paracrine signaling for the treatment of ischemic injury;

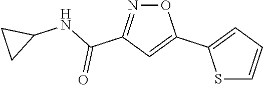 [SMILES: O=C(NC1CC1)c1cc(-c2cccs2)on1]

ISX-9 (aka Neuronal Differentiation Inducer III)

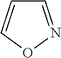

ISOXAZOLE

The end points of these in vivo studies herein were the reversal of fibrosis through angiomyogenic differentiation of the engrafted progenitor cells, functional integration of developing cardiac myocytes and endothelial cells into the host heart, attenuation of infarct size and the functional benefits in terms of improved global heart function.

The initial proof of concept work was both in vivo and ex vivo bench top work, comparing the proliferation of myocytes with exosomes from CPCs alone, with CPCs alone, and with the combination of the two. It was predicted that the combination would prove synergistic which was proven in our data.

Isolated neonatal cardiomyocytes (CMs) we incubated in a petri dish containing the plating medium. The collected cells are then seeded onto collagen-coated tissue culture plates at a density of 2.0×10⁵ cells/well. The plating medium is replaced every other day. CPCs and their exosomes are prepared as described in our prior work.

CPCs alone, exosomes alone, and the combination of the two were added to myocyte culture, and a control group is treated with the base solution lacking either. Proliferating myocytes were then counted.

In more detail, myocytes were exposed to 300 μM H₂O₂ for an hour and then CPS (100 μg) will be added. After an hour, tunnel positive myocytes were be determined. In the second group of myocytes, CPC plus exosomes (100 μg) were added to see its effect on cell death. In the third group, both CPCs and exosomes (200 μg) were added together to the myocytes exposed to H₂O₂ and tunnel positive cells will be determined after an hour. The control group will be buffer only.

It was expected that both CPCs and exosome treatment would enhance proliferation of myocytes. However, it was expected that the combined treatment of CPCs and EX would significantly enhance proliferation in a synergistic manner which was proven. Similarly, the myocytes were protected against oxidizing effects of H₂O₂ by CPCs or exosomes, but the protection anticipated and observed was significantly higher by combined treatment of CPCs with exosomes.

The present disclosure includes any one or more of the following embodiments, in any combination thereof:

A method of treating cardiac disease, comprising administering a composition comprising
allogenic or autologous cardiac progenitor cells (CPCs) plus added extracellular vesicles (in
our cases exosomes)derived from CPCs to a human patient having cardiac disease in an
amount sufficient to treat said cardiac disease.
Any method described herein, wherein said CPCs are derived from said patient.
Any method described herein, wherein said CPCs are derived from said patient by a process
comprising:
i) isolating parent cells from said patient or a person allogenic to said patient, wherein
said parent cells are either induced pluripotent stem cells (iPSCs) or pluripotent stem cells
(PSCs);
ii) treating said parent cells with ISX-9 in an amount effective to induce differentiation into
CPCs.
Any method described herein, wherein said CPCs are derived from said patient by a process
comprising:
i) isolating parent cells from said patient or a person allogenic to said patient, wherein
said parent cells are either induced pluripotent stem cells (iPSCs) or pluripotent stem cells
(PSCs);
ii) culturing said parent cells with 0.1-35 uM ISX-9 for 3-10 days in a medium without
insulin to induce parent cells to form CPCs and then culturing said CPCs in a medium without
ISX-9 and with insulin for 3-10 days to induce differentiation of said CPC cells into
cardiomyocytes or into cardiomyocytes, smooth muscle cells and endothelial cells, and then
using said cardiomyocytes or into cardiomyocytes, smooth muscle cells and endothelial cells in
place of said CPCs in said method.
Any method described herein, wherein said CPCs are subjected to hypoxic preconditioning
before use in said human patient.
Any method described herein, wherein said exosomes comprise miRNA-373 or miRNA-373
and ephrinB2/EphB4 protein(s).
Any method described herein, wherein said exosomes are isolated from a culture of CPCs by
ultracentrifugation, ultrafiltration, precipitation or immunoaffinity capture, or wherein said
exosomes are isolated by centrifugation, filtration, concentration, separation by columns, and
concentration.
Any method described herein, wherein said cardiac disease is a cardiac ischemia or a
myocardial infarction.
Any method described herein, where the composition is administered by intramyocardial
injection.
Any method described herein, said CPC and exosomes in a ratio of about 1/1 to 1/5 or about
1/10.
Any method described herein, wherein 10⁵-10⁷ CPCs and 10⁸-10¹⁰ exosomes are administered
by intramyocardial injection, or 10⁶ CPCs and about 10⁹ exosomes are administered by
intramyocardial injection.
A composition for treating a patient, said composition comprising allogenic or autologous
cardiac progenitor cells (CPCs) plus additional exosomes derived from said CPCs in a
pharmaceutically acceptable carrier.
Any composition herein described, said CPC and exosomes in a ratio of about 1/1 to 1/10
Any composition herein described, said exosomes comprising miRNA-373 or ephrinB2/EphB4
protein.

As used herein, “additional” or “amplified” or an “excess” of exosomes may mean having at least 25% (preferably 50% or 100% more) more exosomes than would naturally be present in a given cell population. Exosomes are added to a CPC population at a level greater than normal.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed and excludes all additional elements.

The phrase “consisting essentially of” excludes additional material elements but allows the inclusions of non-material elements that do not substantially change the nature of the invention.

The following abbreviations are used herein:

| ABBREVIA- | |
|---|---|
| TION | TERM |
| CPC | Cardiac progenitor cell |
| iPSC | Induced pluripotent stem cell |
| hiPSC | Human iPSC |
| ISX-9 | 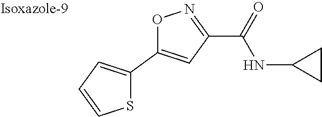 |
| MI | Myocardial infarction |
| PSC | Pluripotent stem cell |
| iPSC | Induced PSC |
| CM | Cardiomyocytes |
| SMC | Smooth muscle cells |
| EC | endothelial cells |
| miR or miRNA | MicroRNA |

### BRIEF DESCRIPTION OF FIGURES

FIG. 1: Overall summary of generation of CPCs from hiPSC for cardiac repair.

FIG. 2: Schematic outline of generation of CPCs.

FIGS. 3A-3C: Characterization of CPCs: Generation and characterization of hiPSC-derived cardiac progenitor cells (CPCs). FIG. 3A, Time-dependent expression of Nkx2.5, GATA4, ISL-1, and Mef2c. *Versus Day 0, P<0.05; #versus Day 3, P<0.05, from three biological repeated experiments. FIG. 3B, Immunofluorescence staining showed that transcription factors (Nkx2.5, GATA4, and ISL-1) were highly upregulated in hiPSC treated with ISX-9. FIG. 3C, Fluorescence activated cell sorting analysis (FACS) showing 96.512.3% Nkx2.5 positive cells after ISX-9 treatment. Scale bar represents 200 mm. GATA4 indicates GATA binding protein 4; hiPSCs, human induced pluripotent stem cells; ISL-1, islet-1; ISX, isoxazole; Mef2c, myocyte enhancer factor-2; Nkx2.5, NK2 Homeobox 5.

FIG. 4: Further differentiation of CPCs into CM, EC and SMC.

FIGS. 5A-5D: Characterization of exosomes: Characterization of EV from iPSC and CPC^{ISX-9}. FIG. 5A Secretion of EV from the CPC^{ISX-9} as imaged by electron microscopy. Bar=1 μm. FIG. 5B EV isolated from iPSC and CPC^{ISX-9} visualized by transmission electron microscopy (TEM). Scale bar=200 nm. FIG. 5C Representative images of western blot for Tsg101, CD9, Hsp70, flotillin-1, and calnexin in EV lysates. C: cell lysate; E: EV. FIG. 5D Representative graph of size distribution of EV from iPSC and CPC^{ISX-9} as detected by TRPS.

FIGS. 6A-6B: FIG. 6A PKH 26 labeled EV from CPC^{ISX-9} (red) were observed inside the fibroblasts (green, Calcein AM), mostly located at the perinuclear region. The white arrows indicated the uptake of EVs. Bar=100 m. FIG. 6B Exosome effect of fibroblasts: Fibrotic gene expression in fibroblasts after TGF-β stimulation. Effects of EV-CPC^{ISX-9} on fibrotic gene expression: role of miR-373. n=6.

FIG. 7: Expression of miRNAs in exosomes: Heatmap analysis of microarray data showing significant upregulation of miRNAs in EV-CPCISX-9 compared with EV-iPSC, EV-EB, or EV-control-CPC. Red or blue colors indicate differentially up- or downregulated miRNA, respectively (P<0:05). n=3.

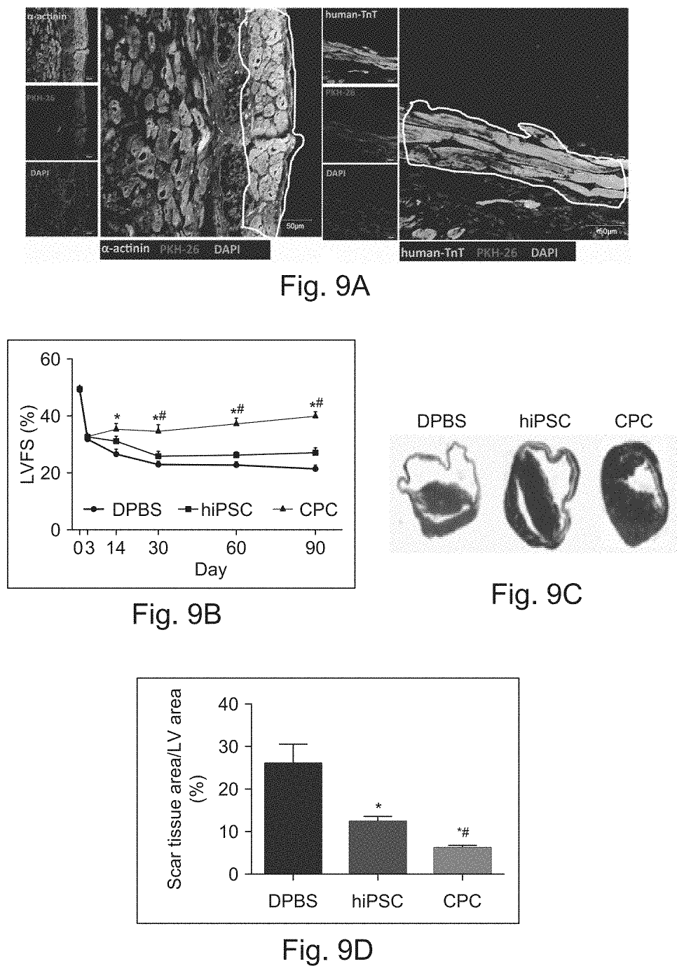

FIGS. 8A-8D: CPC protective effects in ischemia: FIG. 8A Cytoprotective effects of ISX-9 on CPCs. A, Morphology of hiPSCs in RPMI/B27 medium treated with DMSO or ISX-9 under 1% 02 for 12 or 24 h. B, Representative images of TUNEL staining in Mock, DMSO, and ISX-9 treated groups after 24 h hypoxic stresses FIG. 8B Semiquantitative estimate of TUNEL positive cells. *Versus DMSO group, P<0.05. FIG. 8C, Representative images of TUNEL staining 3 days post-MI. The host cardiomyocytes were identified by α-sarcomeric actinin. FIG. 8D, Quantitation of total TUNEL positive cells in the border area of infarct with different treatments. *Versus DPBS group, P<0.05; #versus hiPSC group, P<0.05.

FIGS. 9A-9D: Differentiation of CPCs when injected into the heart: FIG. 9A Engrafted CPCs were identified by PKH-26 fluorescence (red fluorescence); muscle fibers were visualized via immunostaining for human-specific cTnT or α-sarcomeric actinin (Green fluorescence) at 3 M post-MI. FIG. 9B Temporal changes in FS. FIG. 9C cardiac fibrosis was evaluated by 7 levels by Masson Trichrome staining at 3 months' post MI. Sections of the representative heart were shown. FIG. 9D Quantification of scar tissue size. *P<0.05 versus DPBS treated group at the same time point, #P<0.05 versus hiPSC treated group. CPC indicates cardiac progenitor cells; hiPSCs, human induced pluripotent stem cells; MI, myocardial infarction; ISX, isoxazole.

FIGS. 10A-10D: Exosomes promote cardiac repair on injection: CPCISX-9-derived EV promoted cardiomyocyte proliferation and angiogenesis after myocardial infarction (MI) in mice. FIG. 10A Representative image of Ki67-positive cardiomyocytes (cTnT positive) in EV-CPCISX-9-treated mouse hearts 30 days after MI. Bar=50 μm. FIG. 10B Quantitative estimate of proliferating cardiomyocytes as determined by Ki67 staining in the peri-infarct region 30 days after myocardial infarction. PBS group: n=940 cardiomyocytes from 3 hearts; EV-iPSC group: n=950 cardiomyocytes from 3 hearts; EV-CPCISX-9 group, n=951 cardiomyocytes from 3 hearts. * vs. the PBS group, P<0:05; # vs. the EV-iPSC group, P<0:05. FIG. 10C Representative images of arteriole density in the peri-infarct area 4 weeks after MI. Arterioles were identified by α-SMA-positive staining (green) of vascular structures. Bar=100 μm. FIG. 10D Quantitative analysis of arteriole density in different treatment groups.* vs. the PBS group, P<0:05; # vs. the EV-iPSC group, P<0:05, n=3.

FIGS. 11A-11D: CPC^{ISX-9}-derived EV reversed cardiac remodeling in infarcted mice. FIG. 11A Temporal changes in FS. FIG. 11B Quantitative estimate of fibrosis. vs. the PBS group, P<0:05; # vs. the EV-iPSC group, P<0:05, n=4 FIG. 11C Representative M-mode echocardiography images from miRNA mimic negative control- (NC-) treated mice and miR-373 mimic-treated mice 30 days post-MI. FS is shown; n=8 in the NC group and n=7 in the miR-373 mimic group. FIG. 11D Representative Masson's trichrome-stained sections of hearts from NC-treated mice and miR-373 mimic mice. Quantitative analysis of fibrosis post-MI.

FIGS. 12A-12B: Images illustrating FIG. 12A EphrinB2 expression in ISX-9-CPC and FIG. 12B EphB4 expression in normal heart and infarcted heart.

FIGS. 13A-13B: Two graphs FIG. 13A and FIG. 13B illustrating combined treatment of ISX-9-CPC and EX in cardiac function 1 Month post-MI. ISOX-CPC, n=4, ISOX-CPC plus EX, n=3. The administration of CPC's together with their Exosomes improved cardiac function vs. using CPC's alone.

FIGS. 14A-14B: Two graphs FIG. 14A and FIG. 14B illustrating combined treatment of ISX-9-CPC and EX in cardiac function 1 month post-MI.

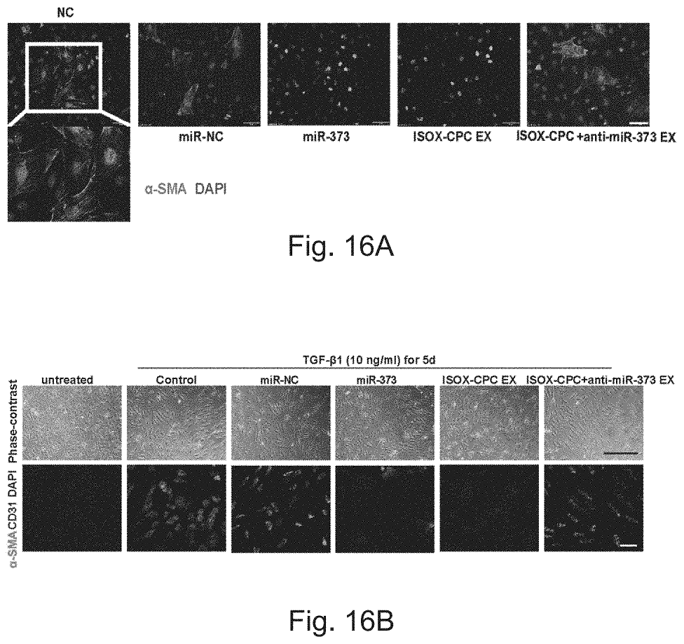

FIGS. 15A-15D: Images illustrating cytoprotection of ISX-9-CPC against ischemia. FIG. 15A In vitro effect on TUNEL staning with 24 h hypoxia (1%02) FIG. 15B Semi-quantitative estimate of TUNEL positive cells. *VS. DMSO group, P<0.05 FIG. 15C In vivo effect on TUNEL positive cells 3 days post-MI. Cardiomyocytes were identified by α-dsrcomeric actinin. Bar=50 μm. FIG. 15D Quantitation of TUNEL positive CM in the border area with different treatments. *vs. PBS groups, p<0.05; # vs. hiPSC groups, p<0.05

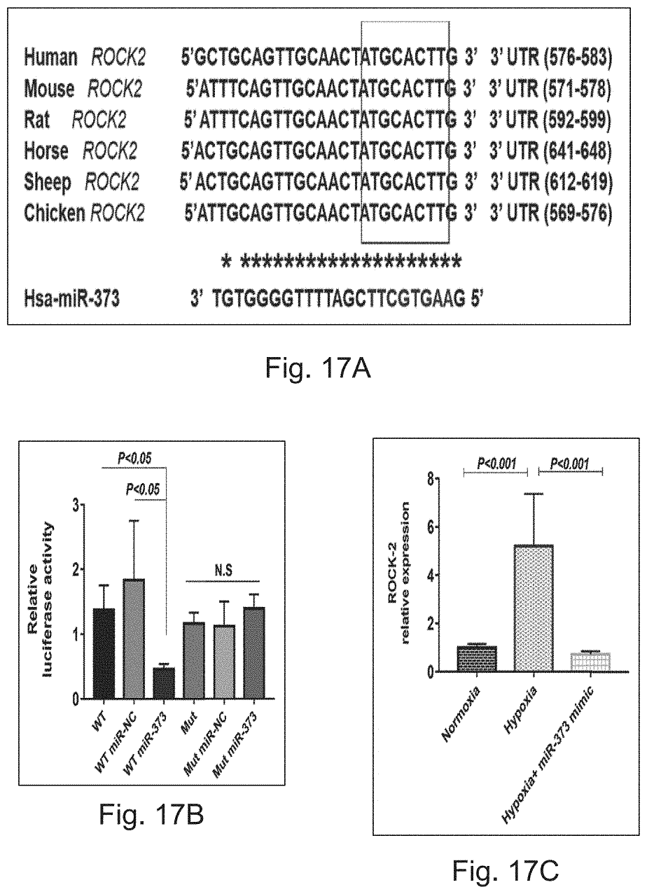

FIGS. 16A-16B: Images illustrating transdifferentiation of fibroblasts into myofibroblasts after 72 h hypoxia. FIG. 16A Transdifferentiation of fibroblasts into myofibroblasts after 72 h hypoxia as shown by immunostaining for a-smooth actin (α-SMA): EX from ISOX-CPC or miR-373 mimic blocked their conversion into myofibroblasts. miR-373 inhibitor abrogated the effect of EX from ISOX-CPC. Bar-501Jm. FIG. 16B Phase-contrast images and immunofluorescence images of CD31 (red) and a-SMA (green)staining in human aortic endothelia cell (HAEC) untreated or treated with 10 ng/ml TGF-β1 for 5d, or in HAEC pretreated with ISOX-CPC EX, miR-373 deficient ISOX-CPC EX or miR-373 mimic (50 nM). ISOX-CPC EX pretreatment significantly inhibited TGF-˜1 induced endothelial-mesenchymal transition (EndMT) with loss of CD31 expression and increase of α-SMA expression, miR-373 inhibitor abrogated such effect. miR-373 mimic partly reversed TGF-β1 induced EndMT.

FIGS. 17A-17C: FIG. 17A Graphs illustrating conservation of human binding sites of ROCK-2 gene with the SEQ ID NO: among species including mice and rat and FIG. 17B relative luciferase activity of 239F T cells FIG. 17C ROCK-2 expression was increased under hypoxia while MIR-373 mimic reduced effects of hypoxia.

FIGS. 18A-18B: Images illustrating FIG. 18A EphrinB2 expression in ISX-9-CPC and FIG. 18B EphB4 expression in normal heart and infarcted heart.

FIGS. 19A-19G: miR-373 mimic improved cardiac function and angiogenesis and attenuated cardiac fibrosis after MI. FIG. 19A Representative M-mode echocardiography images from miRNA mimic negative control- (NC-) treated mice and miR-373 mimic-treated mice 30 days post-MI. FS FIG. 19B and EF FIG. 19C are shown; n=8, in the NC group and n=7 in the miR-373 mimic group. FIG. 19D Representative Masson's trichrome-stained sections of hearts from NC-treated mice and miR-373 mimic mice. FIG. 19E Quantitative analysis of fibrosis post-MI. FIG. 19F Vessel density was assessed by α-SMA-positive staining (green) of vascular structures. FIG. 19G Quantitative estimate of arteriole density. n=3 in each group.

FIGS. 20A-20B: Show the effect of three small molecules (ISX9, Danzol, Givinostat) on expression of cardiac and skeletal muscle genes in FIG. 20A & FIG. 20B. Real time PCR on dystrophin on small molecule (ISX9, GIV) expression in IPS cells in FIG. 20B.

FIGS. 21A-21D show that cardiac fibrobrast derived human iPS cell colonies were dissociated with accutase and plated in the presence of Y27632. FIG. 21A schematically shows cell culture conditions for the generation of cardiac fibroblasts from human iPSCs. Briefly, to generate muscle progenitor cells (MPCs) from hiPSC in vitro, human Induced Pluripotent Stem (iPS) Cells (ATCC® ACS-1021™) induced from human cardiac fibroblasts were cultured with mTeSR™1 (STEMCELL Technologies Inc.) on Vitronectin XF (STEMCELL Technologies Inc.) coated 6-well plates. iPS Cells were passaged every 4 to 6 days with ReLeSR™ (STEMCELL Technologies Inc.). For differentiation of iPS Cells into MPCs, iPS Cells were dissociated into single cells with ACCUTASE™ (STEMCELL Technologies Inc.) into single cells and seeded at 1×10⁵ cells/cm² with mTeSR™1 supplemented with 5 μM RHO/ROCK pathway inhibitor (Y-27632, STEMCELL Technologies Inc.). After 24 hr, the medium was changed to fresh mTeSR™1. mTeSR™1 was refreshed daily during first 3 days. After 3 days, culture medium was changed to mTeSR™1 supplemented with 20 μM ISX-9 (MedChemExpress). The medium was refreshed every other day. After 6 days, the medium was switched to RPMI 1640 Medium (Thermo Fisher Scientific) supplemented with N-2 Supplement (Thermo Fisher Scientific) and 20 μM ISX-9 and refreshed every other day for another 3 to 6 days. Small molecules (Isx9 & GIV) were applied to initiate differentiation and analysed at day 9. FIG. 21B shows relative skeletal muscle gene expression by the treatment of Isx9 & Giv. FIG. 21C and FIG. 21D show the muscle genes (PAX3, PAX7, MYF5, MYOG, MYOD), overexpression superiority in particular of ISX-9.

FIGS. 22A-22E: Characterization of EV from iPSC and CPCISX-9. FIG. 22A Secretion of EV from the CPCISX-9 as imaged by electron microscopy. Inset shows higher magnification of secreted EV (small black arrows). Blue arrows point to EV exiting from the cells. Bar=1 μm. FIG. 22B EV isolated from iPSC and CPCISX-9 visualized by transmission electron microscopy (TEM). Scale bar=200 nm. FIG. 22C Representative images of western blot for Tsg101, CD9, Hsp70, flotillin-1, and calnexin in EV lysates. FIG. 22D Average size of EV as measured by TRPS. No significant difference in average size of EV from iPSC and CPCISX-9 was observed. FIG. 22E Representative graph of size distribution of EV from iPSC and CPCISX-9 as detected by TRPS.

FIGS. 23A-23D: miRNA expression profiling and validation of microarray data. miRNA expression profiling and validation of microarray data. FIG. 23A Outline of experimental procedure. FIG. 23B Heatmap analysis of microarray data showing significant upregulation of miRNAs in EV-CPCISX-9 compared with EV-iPSC, EV-EB, or EV-control-CPC. Red or blue colors indicate differentially up- or downregulated miRNA, respectively (P<0:05). n=3. FIG. 23C Biological process of Gene Ontology (GO) enrichment analysis based on miRNA-targeted genes. GO enrichment was analyzed with mirPath v.3 software. GO biological process includes biological processes, molecular function, and cellular component of upregulated and downregulated genes. FIG. 24D Validation of microarray data using real-time PCR. Quantitative results showing significant expression of miR-373, miR-367, miR-520, miR-548ah, and miR-548q in EV-CPCISX-9. RNA samples were from three individual experiments. *P<0:001.

FIGS. 24A-24G: Fibrotic gene expression in fibroblasts after TGF-β stimulation. Fibrotic gene expression in fibroblasts after TGF-β stimulation. FIG. 24A Effects of EV-CPCISX-9 on fibrotic gene expression: role of miR-373. n=6. FIG. 24B Transdifferentiation of lung fibroblasts and dermal fibroblasts into myofibroblasts by hypoxia for 72 has detected by immunostaining for α-smooth actin (α-SMA): effects of EV-CPCISX-9 and miR-373 mimic pretreatment. Bar=50 μm. FIG. 24C Schematic representation of the luciferase reporter constructs. FIG. 24D Sequence alignment of miR-373 with the human wild-type (WT) ROCK-2 3′-UTR and GDF-11 3′-UTR and mutated reporters. The seed sequence (red) is highlighted. FIG. 24E Relative luciferase activity (relative, firefly luciferase activity/Renilla luciferase activity) of 293FT cells cotransfected with WT 3′-UTR-ROCK-2 or GDF-11 and mutant 3′-UTR-ROCK-2 or GDF-11 and miR-373 mimics vs. NC. **P<0:01, n=4. UTR: untranslated region; miRNA: microRNA; NC: negative control; WT: wild type. FIG. 24F 72 h hypoxia increased GDF-11 and ROCK-2 mRNA expression in lung fibroblasts: effects of pretreatment with miR-373 mimic. ***P<0:001. n=6. FIG. 24G Pretreatment of fibroblasts during hypoxia with EV-CPCISX-9 significantly decreased the upregulation of GDF-11 and ROCK-2 similar to pretreatment with miR-373 mimic. ***P<0:001. n=6.

FIGS. 25A-25D: CPCISX-9-derived EV promoted cardiomyocyte proliferation and angiogenesis after myocardial infarction (MI) in mice. FIG. 25A Representative image of Ki67-positive cardiomyocytes (cTnT positive) in EV-CPCISX-9-treated mouse hearts 30 days after MI. Bar=50 μm. FIG. 25B Quantitative estimate of proliferating cardiomyocytes as determined by Ki67 staining in the peri-infarct region 30 days after myocardial infarction. PBS group: n=940 cardiomyocytes from 3 hearts; EV-iPSC group: n=950 cardiomyocytes from 3 hearts; EV-CPCISX-9 group, n=951 cardiomyocytes from 3 hearts. * vs. the PBS group, P<0:05; # vs. the EV-iPSC group, P<0:05. FIG. 25C Representative images of arteriole density in the peri-infarct area 4 weeks after MI. Arterioles were identified by α-SMA-positive staining (green) of vascular structures. Bar=100 μm. FIG. 25D Quantitative analysis of arteriole density in different treatment groups. * vs. the PBS group, P<0:05; # vs. the EV-iPSC group, P<0:05, n=3.

FIGS. 26A-26G: CPCISX-9-derived EV reversed cardiac remodeling in infarcted mice. FIG. 26A Representative M-mode echocardiography images from three groups 30 days after MI. LVDs FIG. 26B, LVDd FIG. 26C, EF FIG. 26D, and FS FIG. 26E are shown. * vs. the PBS group, P<0:05; # vs. the EV-iPSC group, P<0:05. PBS group: n=10, EV-iPSC group: n=9, and EV-CPCISX-9 group: n=11. EF: ejection fraction; FS: fractional shortening; LVDd: diastolic left ventricular dimensions; LVDs: systolic left ventricular dimensions. FIG. 24F Representative Masson's trichrome-stained sections of hearts from the three groups. FIG. 24G Quantitative estimate of fibrosis. * vs. the PBS group, P<0:05; # vs. the EV-iPSC group, P<0:05,n=4.

FIGS. 27A-27G: miR-373 mimic improved cardiac function and angiogenesis and attenuated cardiac fibrosis after MI. FIG. 27A Representative M-mode echocardiography images from miRNA mimic negative control- (NC-) treated mice and miR-373 mimic-treated mice 30 days post-MI. FS FIG. 27B and EF FIG. 27C are shown; P<0:001, n=8 in the NC group and n=7 in the miR-373 mimic group. FIG. 27D Representative Masson's trichrome-stained sections of hearts from NC-treated mice and miR-373 mimic mice. FIG. 27E Quantitative analysis of fibrosis post-MI. FIG. 27F Vessel density was assessed by α-SMA-positive staining (green) of vascular structures. Bar=100 μm. FIG. 27G Quantitative estimate of arteriole density. P<0:05. n=3 in each group. Schematic depiction of mechanisms of protection by EV-CPCISX-9: role of miR-373 in suppressing fibrosis by targeting two genes, GDF-11 and ROCK-2, and inhibiting myofibroblast differentiation. Myocyte proliferation and angiogenesis were also promoted by EV-CPCISX-9

FIG. 28: Schematic depiction of mechanisms of protection by EV-CPCISX-9: role of miR-373 in suppressing fibrosis by targeting two genes, GDF-11 and ROCK-2, and inhibiting myofibroblast differentiation. Myocyte proliferation and angiogenesis were also promoted by EV-CPCISX-9.

FIGS. 29A-29C: Generation of muscle progenitor cell (MPC) from human iPSC using small molecules FIG. 29A Schematic outline of generation of MPC from human PSC using combination of CHIR99021 and givinostat or CHIR99021 only. FIG. 29B Morphology of differentiating cells from 4 human iPSC lines (CF-iPSC, DF-iPSC-1, DF-iPSC-2 and DMD-iPSC) at 7 days. Bar=200 μm. FIG. 29C Morphology of replated MPC and differentiated myotubes from 4 human iPSC lines at day 14. Bar=200 μm.

FIGS. 30A-30B: Characterization of givinostat-induced MPC. Characterization of givinostat-induced MPC. FIG. 30A The treated hiPSC at day 14 expressed Pax7 and desmin. FIG. 30B The differentiated myotubes expressed MF20 as shown by immunostaining. Bar=50 μm.

FIGS. 31A-31H: Givi-MPC exhibited superior proliferation and migration capacity. FIG. 31A Representative images and quantitative estimate FIG. 31B of cell migration by adult human myoblasts, and control-MPC, Givi-MPC (arrow). Cells were stained with Calcein AM (green). Bar=1 mm. FIG. Quantitative estimate of migrated cells. Givi MPC showed highest number of cells migrated compared with human myoblasts (P<0.0001) or CHIR99021 induced MPC (P<0.0001). No significant difference was observed between human myoblasts and control-MPC. FIG. 31C Heat map of the Human RT2 motility PCR Array. FIG. 31D Differentially expressed genes related to migration in Givi-MPC vs. control-MPC using human cell motility PCR array. FIG. 31E Proliferation curves of human myoblasts vs MPC using CCK-8 assay. *P<0.05; #P<0.05 vs. control-MPC. n=6. FIG. 31F Morphology of MPC colonies. Bar=500 m. Number of colonies FIG. 31G and percentage of colonies with different cell number in color FIG. 31H control-MPC: CHIR99021 induced MPC; Givi-MPC: CHIR99021 and Givinostat induced MPC.

FIGS. 32A-32E: In vivo myogenic potential of different MPC and myoblast in Mdx/SCID mice with CTX injury. FIG. 32A Dystrophin restoration in Mdx/SCID mice by MPC transplantation at 1M after CTX injury. Bar=50 μm. FIG. 32B Transplanted cells were labeled with GFP (Green) and identified with human laminin staining (Red). Quantitation of engrafted fibers at 1M: Dystrophin+fibers (n=6) FIG. 32C and human laminin and GFP double positive fibers (n=3) FIG. 32D & FIG. 32E Cross-section showing pre-synaptic staining with α-bungarotoxin in dystrophin positive fibers (n=3). Bar=20 μm.

FIGS. 33A-33E: Givi-MPC decrease inflammation and muscle necrosis in Mdx/SCID mice 7 days after CTX injury. FIG. 33A Representative images of HE and Trichrome Masson staining in Mdx/SCID mice with human myoblasts or control-MPC or Givi-MPC transplantation 7 days after CTX injury. Black arrows indicate infiltrated inflammatory cells. FIG. 33B Quantification of muscle fiber necrosis between PBS treated collateral TA muscle or Givi-MPC treated TA muscle 7 days after CTX injury. FIG. 33C Quantification of muscle fiber necrosis of TA muscle among human myoblast or control-MPC or Givi-MPC transplantation mice 7 days after CTX injury. FIG. 33D Quantification of CD68 positive cells in TA muscle following MPC transplantation 7 days after CTX injury. FIG. 33E Representative images of macrophages (red, CD68) and human cells in TA muscle of Mdx/SCID mice with CTX injury following MPC transplantation.

FIGS. 34A-34J: Givi-MPC decrease muscle necrosis and fibrosis in Mdx/SCID mice 1M after CTX injury. FIG. 34A Representative images of HE and Trichrome Masson staining in Mdx/SCID mice after transplantation with human myoblasts or control-MPC or Givi-MPC 1M after CTX injury. Bar=500 μm (4×) and Bar=100 μm (20×). Quantification of necrotic muscle fibers after treatment with human myoblasts FIG. 34B control-MPC FIG. 34C and Givi-MPC FIG. 34D 1M after CTX injury. FIG. 34E Comparison of muscle necrosis among human myoblasts or control-MPC or Givi-MPC transplantation mdx/SCID mice. FIG. 34F Representative images of tissue stained with Sirius red from Mdx/SCID mice. Bar=100 μm. Quantification of muscle fiber fibrosis in collateral. TA muscle treated with human myoblasts FIG. 34G, control-MPC FIG. 34H and Givi-MPC FIG. 34I 1M after CTX injury. FIG. 34J Muscle fibrosis after transplantation of different MPCs in mdx/SCID mice.

FIGS. 35A-35D: Givi-MPC repopulated the muscle stem cell pool. FIG. 35A Muscle cells positive for Pax7 (green) and human nuclear antigen (red) cell under the basal lamina from Mdx/SCID mice after 1M of Givi-MPC transplantation. Bar=20 μm. FIG. 35B Schematic of reinjury experiment. FIG. 35C 1M after reinjury, expression of dystrophin in Givi-MPC treated TA muscle tissue and contralateral PBS treated TA muscle tissue. Bar=50 μm. FIG. 35D Representative HE stained images of Givi-MPC treated TA muscle tissue and contralateral PBS treated TA muscle tissue. Bar=50 μm.

FIGS. 36A-36E: Extracellular vesicles derived from Givi-MPC promoted angiogenesis. FIG. 36A Representative images of CD31 (Red) and laminin (Green) staining in Mdx/SCID mice 1M post injury. Bar=50 μm. FIG. 36B Quantification of capillary density (CD31 positive capillaries). FIG. 36C Representative images of tube formation by human aortic endothelia cells (HAECs) following EV treatment from human myoblasts, or control-MPC or Givi-MPC (1 μg/well, 24 well plate). HAECs were labeled with Calcein AM (Green). Bar=500 μm. FIG. 36D Tube formation assay. Average tube length was analyzed from 3 biological repeated experiments. FIG. 36E Heatmap showing significant upregulation of miRs in EV derived from Givi-MPC compared to EV-human myoblasts.

FIG. 37: Generation of muscle progenitor cell (MPC) from human iPSC using small molecules. Schematic outline of control-generation of MPC from human PSC using combination of CHIR99021 and givinostat or CHIR99021 only.

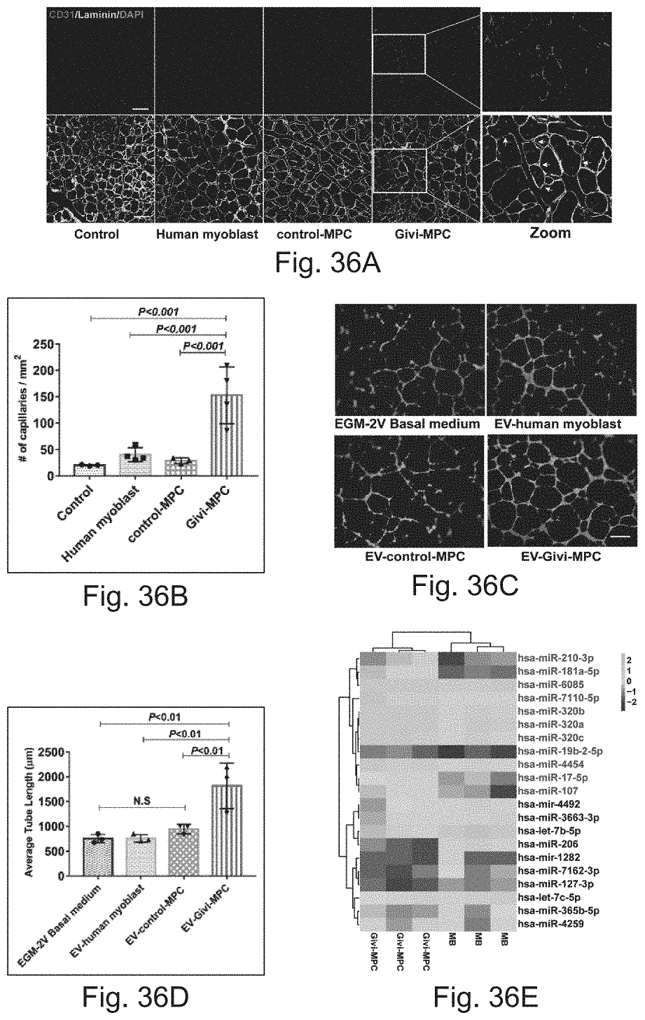

FIG. 38 Engrafted Givi-MPC (GFP positive) expressed dystrophin. Bar=200 μm.

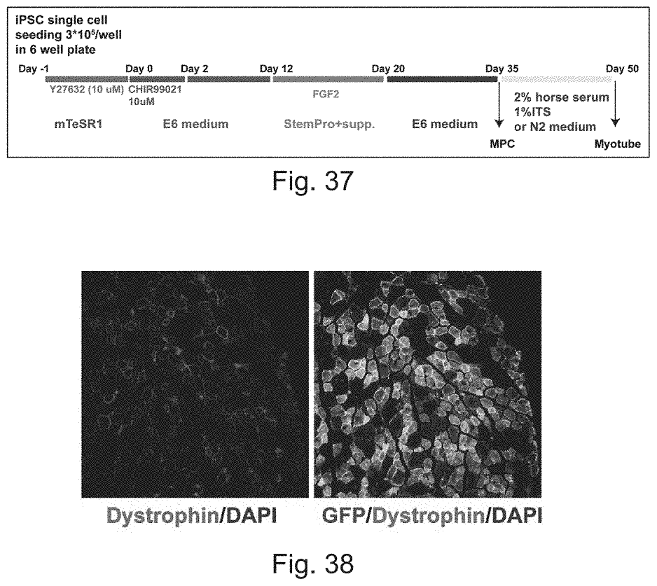

### DETAILED DESCRIPTION OF PERFORMED EXPERIMENTS

ISX-9 is a small molecule which has been obtained from StemCell Technologies. Each reagent was aliquoted and stored per manufacturers' guidelines and under qualified supervision. Antibodies, buffers, and primers were ordered from the manufacturer catalog number and stored per manufacturer recommendations. Chemicals were authenticated by liquid chromatography.

Induced pluripotent stem (iPS) cells may hold therapeutic promise for cardiovascular diseases. The success of effective cell based therapy may lie towards generation of cardiovascular progenitors which may allow successful regeneration of infarcted tissue and replace scar tissue will fully functional myocytes integrated with host myocardium without the risk of tumor formation\[1]. The transplanted stem cells are known to differentiate into cardiac lineage cells in a cardiac ischemic environment and improve cardiac function.

Cardiac progenitor cells (CPCs) may offer a promising avenue for cardiac repair due to their multipotency and ability to proliferate. Efficient cardiac-lineage priming with small molecules prior to hiPSC or hESC differentiation decreases not only risk of tumorgenecity by reducing numbers of undifferentiated cells, but may also limit risk of immune rejection. Besides differentiation they release multiple factors involved in anti-inflammatory, fibrotic, apoptotic, and remodeling processes and rescues the injured myocardium[2], the paracrine mechanisms may involve the release of cytokines, chemokines, and exosomes[3-9, 98].

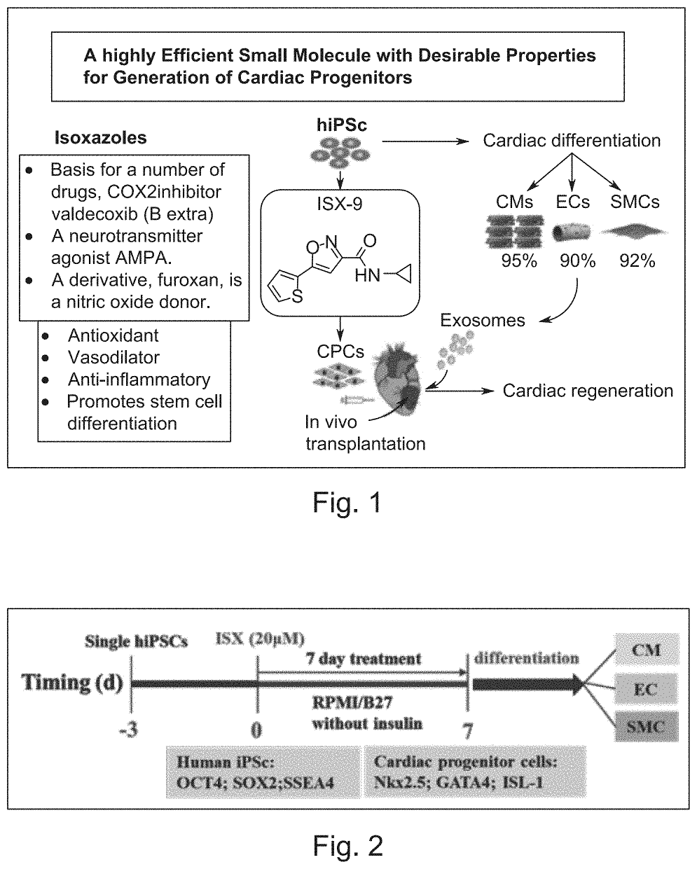

Recent discovery of exosomes for paracrine factors may prompt new approaches to accelerate the process of regeneration together with CPCs. These progenitors were assessed for therapeutic benefits in pre-clinical mouse model. These studies were intended to generate a significant quantity of multipotent progenitor cells from iPS and their exosomes for restoring damaged myocardium without the risk of tumor formation (FIG. 1). The main hypothesis of the proposal was that derived cardiovascular progenitors supplemented with their exosomes will be more effective in successful regeneration of infarcted myocardium without the risk of tumorgenicity and immune rejection.

The initial thesis was that Human iPSC reprogrammed CPCs with a “specific small molecule” possessing anti-oxidative, anti-inflammatory and cardiac gene promoting properties will maximize regenerative capacity of IPSC and their derivatives. To this end, efforts associated with the initial thesis we differentiated hiPS cells with a small molecule into cardiac lineage cells.

A highly efficient single small molecule, isoxazole-9 (ISX-9) capable of transforming hiPSC into cardiac lineage cells has been identified. These CPCs are multipotent and highly proliferative and meet the needs of modern regenerative medicine. These CPCs act through anti-inflammatory, immunomodulatory, pro-survival and anti-fibrotic mechanisms [10, 11].

A novel, cell free CPC based therapeutic approach utilizing paracrine signaling for the treatment of ischemic injury was then developed. Molecular and biochemical properties of exosomes are regulated by the stem cell source and environment of their tissue of origin. Exosomes can be generated in large numbers by highly proliferative and multipotent CPCs and can salvage the infarcted heart[98]. Then identified, purified, and analyzed were the exosome cargo from ISX-9-CPCs and non ISX-9-CPCs; Furthermore, associated efforts also: determined the miRNA profile in exosomes from ISX-9-CPCs and non ISX-9-CPCs. determined the efficacy of the endogenous repair of the hypoxia-injured CPCs, cardiac myocytes and endothelial cells by exosomes and miRNA mimics and also showed that anti-apoptotic and proliferative properties promoted by ISX-9 in CPCs are expressed in exosomes[98].

We also performed hypoxic preconditioning which to enhanced the release of specific cardiogenic miRs (ie MIR-373) and bioactive proteins via exosomes to stimulate regeneration which was also previously reported in our publication[98].

Also studied was the therapeutic efficacy of ISX-9 CPCs vs non ISX-9 CPCs and their exosomes in murine MI model.

Also identified was that the molecular mechanism are cardioprotection by exosomes derived from CPCs.

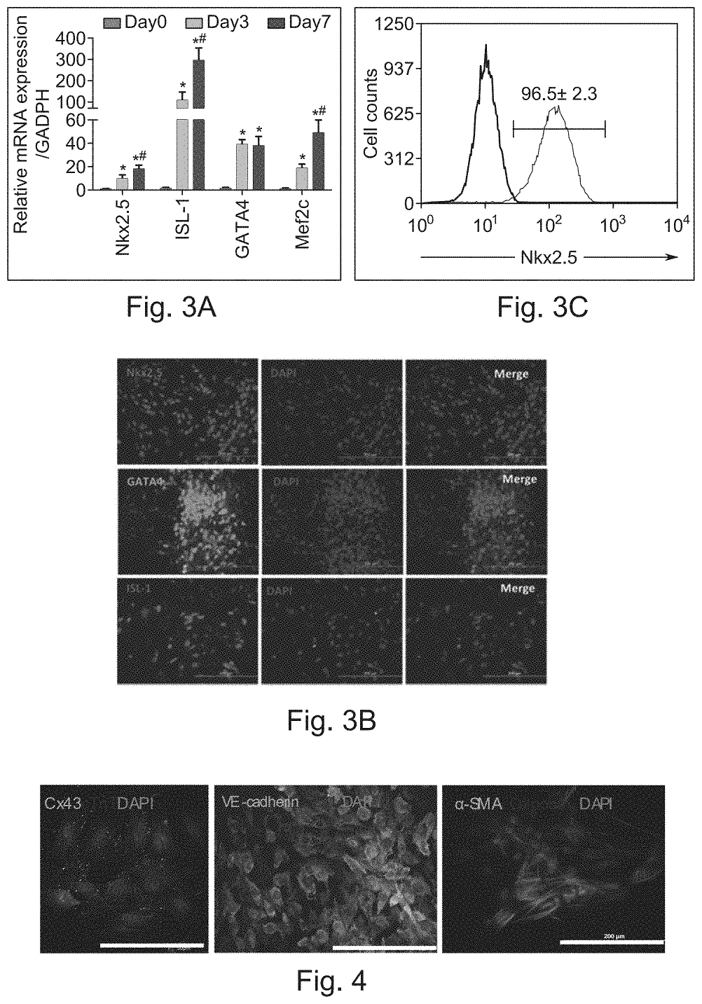

The small molecule N-cyclopropyl-5-(thiophen-2-yl)-isoxazole-3-carboxamide (ISX-9) has been shown to induce cardiac lineage priming in adipose-derived stem cells, which can be differentiated into CM that improve heart function when transplanted in the mouse model of myocardial infarction[23]. It is unique chemical with diverse properties of altering gene expression. A modified ISX-9 molecule forms hydrogels in vitro that bind many RNAs and RNA-binding proteins[24, 25], as ISX-9-like compounds have the potential to elicit broad-sweeping changes in mRNA stability and gene expression. This single compound was found in instances to initiate cardiac reprogramming of iPSC into CPCs expressing cardiac transcription factors within a week (FIG. 2).

We have previously shown that human iPSC derived and reprogrammed CPCs with a “specific small molecule” possessing anti-oxidative, anti-inflammatory and cardiac gene promoting properties will have greater efficacy in cardiac regeneration which was shown[1,98].

We also differentiated hiPS cells with a “specific small molecule” ISX-9 into cardiac lineage cells.; Induced pluripotent stem cells (hiPSCs) can proliferate indefinitely in an undifferentiated state and transform into many cell types in human tissues, including the heart. Therefore, hPSCs are potentially useful in cell-based therapies for heart disease. Multiple cardiac differentiation methods have been described and these procedures need animal cells, fetal bovine serum (FBS), or various cytokines.

Cardiac progenitor cells (CPCs) offer a promising avenue for cardiac repair due to their multipotency and ability to proliferate. The CPCs possess higher proliferative capacity than differentiated CMs and survive better after transplantation due to their earlier developmental stages and their relatively low demand for oxygen.

Data has shown that the overall regeneration efficiency of CPCs may be better than that of CMs because of CPC's multipotency to differentiate into CMS and vessel cells. Current strategies of human CPCs generation include isolation from atria appendage of donors and expansion in vitro[35], derivation from hiPSCs or ESCs, which have to be converted into embryoid bodies or treated with multiple small molecules and growth factors (activin, BMP4)[36, 37].

Such current strategies are labor-intensive and time-consuming, with high production costs, which limit clinical application. Despite these weaknesses, efficient cardiac-lineage priming with small molecules prior to hiPSC or hESC differentiation may decrease not only risk of tumorgenecity by reducing numbers of undifferentiated cells, but may also limit risk of immune rejection.

Small molecules can also substitute for recombinant cytokines and unknown factors in serum[38]. A number of small molecules have been examined or screened for promotion of differentiation: a BMP signaling inhibitor, a p38MAPK signaling inhibitor, a WNT signaling activator, and WNT signaling inhibitors were all reported to promote cardiac differentiation[39-43]. More recently, functional cardiomyocyte-like cells can be generated by treating human fibroblasts with a combination of nine compounds (9C) without genetic material[44]. Amongst small molecules, ISX-9 is a unique small molecule with the ability to trigger multiple signaling pathways[47] leading to conversion of iPSC into different lineage cells.

Here, based on data it was proposed that ISX-9 strongly induces expression of mesodermal and ectodermal fates leading to formation of cardiac progenitors capable of cardiac regeneration. These CPCs exhibit spontaneous contraction within 10 days after induction.

We had two experimental designs whereby in Group 1: Commercial CPCs obtained from iCell or generated using published techniques[1,98]; Group 2: hiPSC line+ 10-20 uM ISX-9; Group 3: validation in additional hiPSC lines using ISX-9.

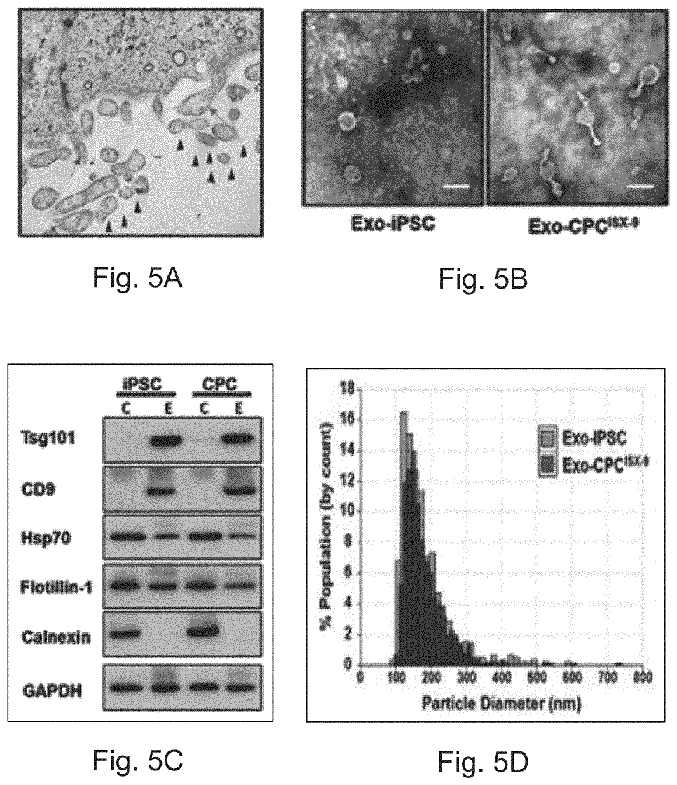

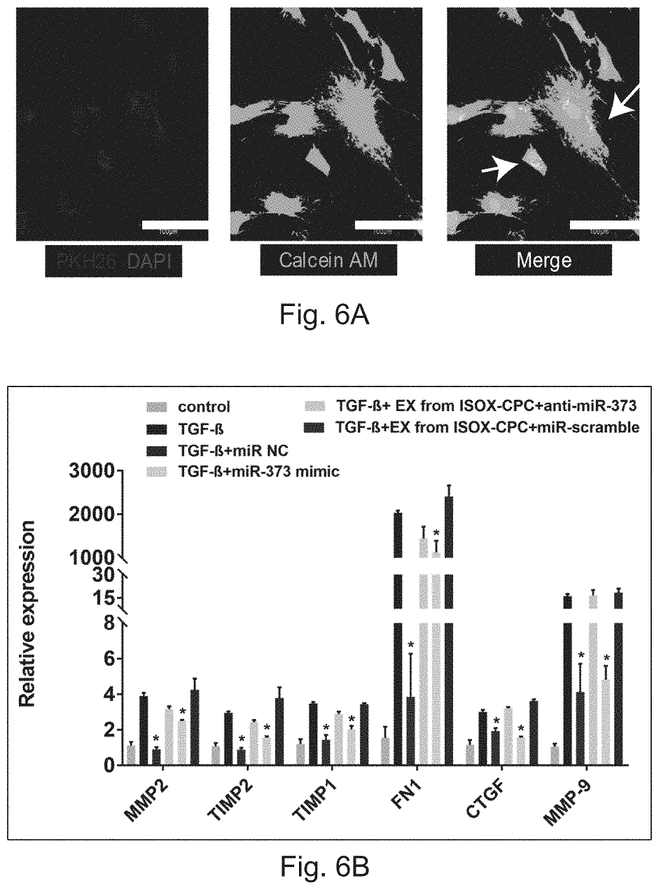

Data showed: hiPSCs cell line (ACS-1021™) was used to generate CPCs with the treatment of ISX-9 as outlined in FIG. 1-2. hiPSCs cultured in mTeSR1 were dissociated into single cells using accutase (Invitrogen) at 37° C. for 10 min and then were seeded on to a vitronectin-coated six-well plate at 1×10⁶ cell/well in mTeSR1 supplemented with 5 uM ROCK inhibitor (Y-27632, Stem Cell Technology) for 24 h. The medium was changed to RPMI/B27 minus insulin supplemented with ISX-9 (20 uM, dissolved in DMSO) for 7 days. Within 3-day ISX-9 treatment, Nkx2.5, GATA4, ISL-1 and Mef2c are upregulated (FIG. 3). By day 7 of ISX-9 treatment cells were 96.5 2.3% Nkx2.5 positive (FACS) and were multipotent and directly differentiated into all three cardiovascular lineages, including CMs, ECs and SMCs in basal differentiation conditions without any specific induction signaling molecules (FIG. 4). For endothelial cells (ECs) differentiation, culture medium was switched to EGM-2V medium (Lonza) for another 10 days. For smooth muscle cells (SMCs) differentiation, culture medium was replaced by DMEM-F12 medium supplemented with TGFβ (2 ng/ml, R&D) and PDGFBB (10 ng/ml, R&D) for 10 days.

Efforts described herein differentiated hiPSC cell lines with ISX-9 into cardiac progenitor cells and their derivatives (FIG. 2): hiPSCs cultured in mTeSR1 were dissociated into single cells using accutase (Invitrogen) at 37° C. for 10 min and were seeded on to a vitronectin-coated six-well plate at 1×10⁶ cell/well in mTeSR1 supplemented with 5 μM ROCK inhibitor (Y-27632, Stem Cell Technology) for 24 h. Afterwards cells were cultured in mTesR1, and changed daily. At day 0, the medium is changed to RPMI/B27 minus insulin supplemented with ISX-9 (20 μM, dissolved in DMSO) for 7 days. Next, for CMs differentiation, after 7-10 days of ISX-9 treatment, culture medium was switched to RPMI/B27 with insulin for another 10 days. Long term cultured CM were maintained in STEMdiff™ Cardiomyocyte Maintenance medium. For endothelial cells (ECs) differentiation, culture medium was switched to EGM-2V medium (Lonza) for another 10 days. For smooth muscle cells (SMCs) differentiation, culture medium was replaced by DMEM-F12 medium supplemented with TGFβ (2 ng/ml, R&D) and PDGFBB (10 ng/ml, R&D) for 10 days.

For ISX-9-CPCs characterization, mRNA-Sequencing transcriptome analysis and global miRNA expression profiles analysis was performed. Expression of cardiac transcription factors, such as GATA4, Mef2C, ISL-1, Nkx2.5 were analyzed by RT-PCR, immunostaining and FACS[FIG. 3].

Further characterized were ISX-9-CPCs derived CMs with cardiomyocyte markers by immunofluorescence, sarcomeric structure by transmission electron microscopy and intracellular electrical recording of action potentials from single beating CM by patch clamp.

We were able to obtain fully reprogrammed iPSC over 90% expressing CPCs markers. These were consistent and reproducible results over several repeats.

A novel, cell free CPC based therapeutic approach utilizing paracrine signaling for the treatment of ischemic injury was further developed.

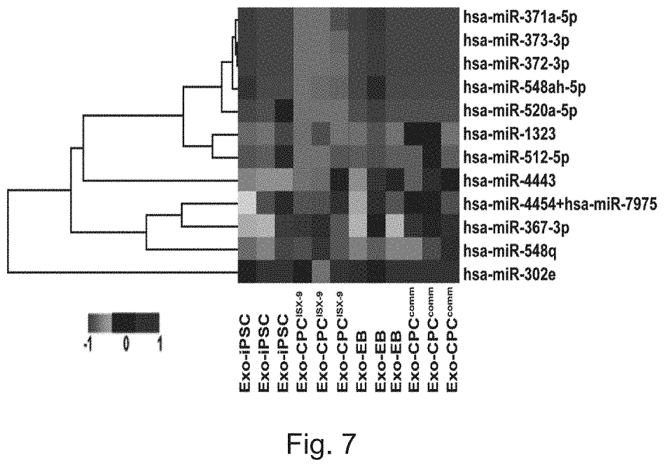

Cell free exosomes (Ex) were generated in large numbers by highly proliferative and multipotent CPCs and these can salvage the infarcted heart. Two kinds of exosomes from CPCs were compared; exosomes from isoxazole initiated CPCs (ISX-9 CPC) vs non ISX-9 CPCs as we observed superior results due their anti-inflammatory, anti-apoptotic and proliferative properties promoted by isoxazole in CPCs compared to non ISX-9 CPCs. Exosomes are small microvesicles, 30-200 nm in diameter, and are stored within multivesicular bodies and released into the environment by fusion with the cell membrane (FIG. 5)[49, 50].

Exosomes are produced by all cells and possess adhesion molecules on their surface which may guide to target delivery of their cargo into specific cell types. They contain a distinct cargo that not only represents the cell of origin but may also be differentially-enriched in specific nucleic acid or lipid species[51]. Integrin activation, sonic hedgehog signaling, and microRNA transfer are among the possible mediators for exosome-induced biological effects[9]. exosomes are internalized as intact vesicles in target cells[52] or EVs fuse with target cells and dump their bioactive cargo (specifically microRNAs), which in turn alters the transcriptome potential of the target cells[53]. EV-target cell interaction is restricted to merely surface interaction[54].

Exosomes internalization by EC[55] have been shown. Since miRs are the major components of exosomes that regulate the function of target cells[56], plans include investigation of the differential expression of specific miRNAs residing within CPC (ISX-9, non ISX-9) exosomes vs. hiPSC by microarray and deep sequencing screening.

Also determined were the miRNA profile in exosomes from ISX-9-CPCs and non ISX-9-CPCs both under normoxic and hypoxic conditions.

We also showed that certain anti-apoptotic and proliferative properties promoted by isoxazole in CPCs are also expressed in exosomes.

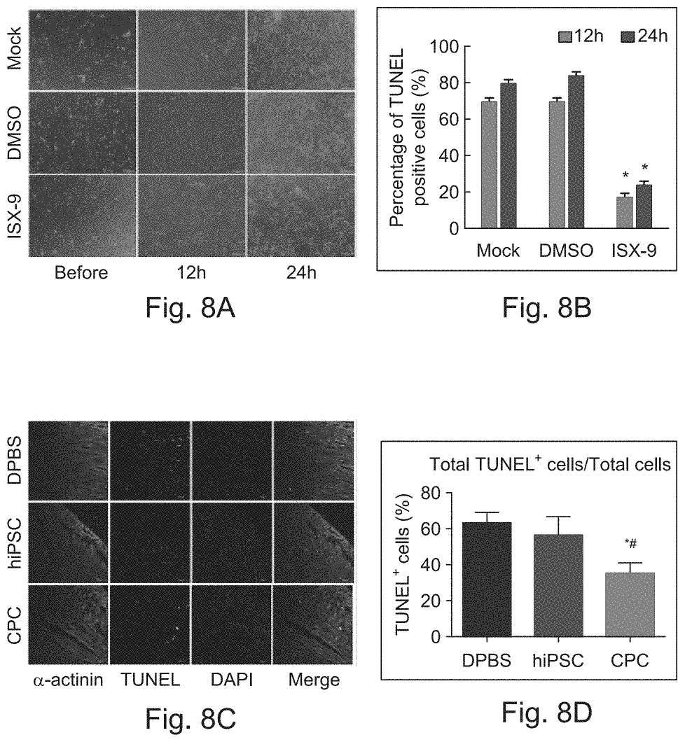

Data support the notion that the endogenous exosomes generated from multipotent, proliferative, regenerative CPCs exert potentially anti-fibrotic effects by transferring their exosomes to cardiac fibroblasts (FIG. 6,[58]).

CPC-derived exosomes represent a mechanism of action of progenitors to enable endogenous self-repair of the damaged hearts by cell to cell transfer of proteins, mRNAs, and miRNAs. Data demonstrates that the primary mechanism of myocardial restoration by cardiac progenitors is both regeneration and paracrine and that the exosomes effectively salvage the injured myocardium. There is direct link/correlation between ISX-9 derived CPCs and their exosomes in their action to promote endogenous repair.

We also determined the miRNA profile of exosome cargo from ISX-9-CPCs and non ISX-9-CPCs

ISX-9-CPCs and non-ISX-9-CPCs were generated as described[1, 48, 98]. Exosomes were purified from the media by qVE size exclusion column following standard protocols, visualized by transmission electron microscopy, and western blot (FIG. 5) and conjugated to 3.92-um latex beads for flow cytometric confirmation of exosomal surface marker CD63. Exosome particle number were then quantified using qNano (IZON). There were differences in protein and/or RNA content of the different exosome populations which we compared.

Then quantified were the miRNA expression with a two-step polymerase chain reaction (PCR) process hybridized to microarrays with probes to hundreds of miRNA targets and protein contents by proteomic analysis.

Then used was a ultracentrifugation approach to copurify many other extracellular species such as protein aggregates and other vesicle types[21] which can cause inflammatory response. then were purified exosomes from other extracellular vesicles (EVs) and large protein aggregates through ultracentrifugation.

CPCs are multipotent cells capable of forming cardiac cells including myocytes, endothelial cells. These are highly proliferative and have the ability to regenerate the ischemic myocardium.

Treatment consisted of plated or 1×10⁸ of iCPCs: 1) 1*10⁸ non ISX-9-CPCs exosomes/well, 2) 1*10⁸ ISX-9-CPCs exosomes/well, 3) non ISX-9-CPCs miRNA mimic (selected from highly expressed miR) (50 nM), 4) ISX-9-CPCs miRNA mimic (selected from highly expressed miR) (50 nM), and 5) CPCs culture medium (control).

The following data was obtained: To determine which miRNA is involved in proliferation, CMs were treated with specific miRNA-373 mimic particle/compound. The number of proliferating CMs were compared amongst groups.

A protocol was developed to purify these vesicles, free from extracellular protein components and other vesicles secreted by the same cells or present in the media. Therefore, it was expected that characterization of exosome size and charge by TEM, cryo-EM, and TRPS accurately reflects the properties of the exosome populations being studied.

Further showed was that anti-apoptotic and proliferative properties promoted by isoxazole treatment in CPCs are expressed in exosomes compared to non ISX-9 CPCs.

Hypoxic preconditioning enhances release of cardiogenic miRs and bioactive proteins via exosomes to stimulate regeneration.

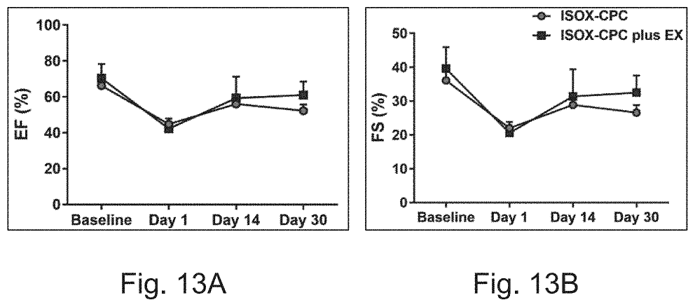

Studies strongly suggested specific exosomes released from the treatment of iPSC with ISX-9 included anti-fibrosis miRs important in attenuating fibrosis (FIG. 11). In this study it was hypothesized that HPC enhances survival and regeneration by CPCs by secreting bioactive molecules and cardiogenic miRs via exosomes. Isoxazole, a small molecule with biologically potent properties[1,98] was used in this research.

Stem cell therapy may offers hope for cardiac tissue repair and regeneration following heart attack. Exosomes are regarded as the critical agents of cardiac regeneration triggered by stem cells. Exosomes are nano-sized biological membrane-enclosed vesicles (30-200 nm) that contain a cell-specific cargo of proteins, lipids, and nucleic acids and act as mediators of cell-cell communication.

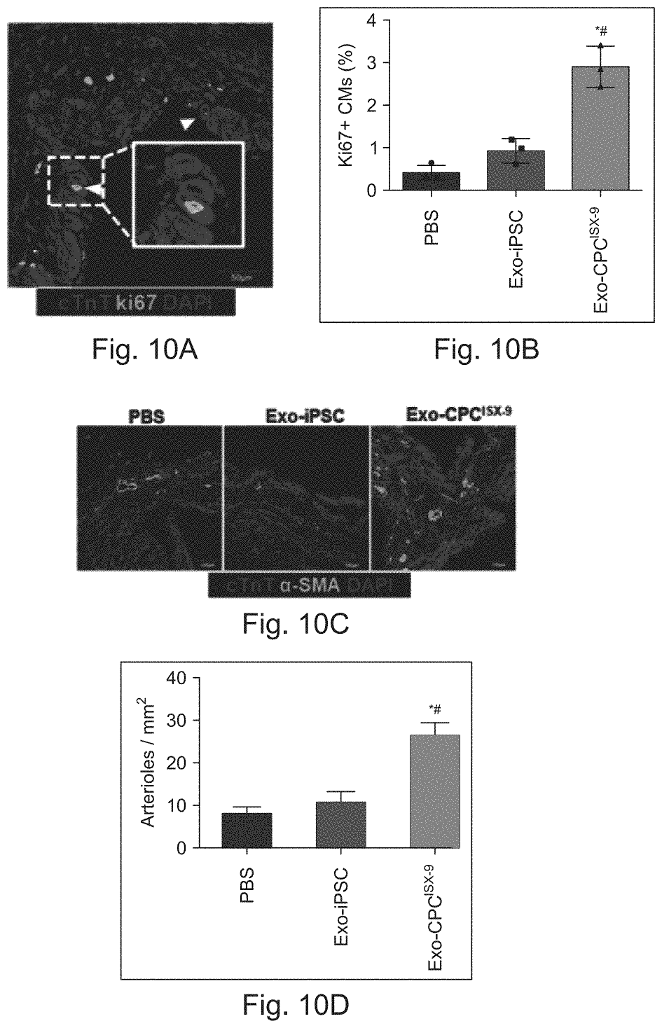

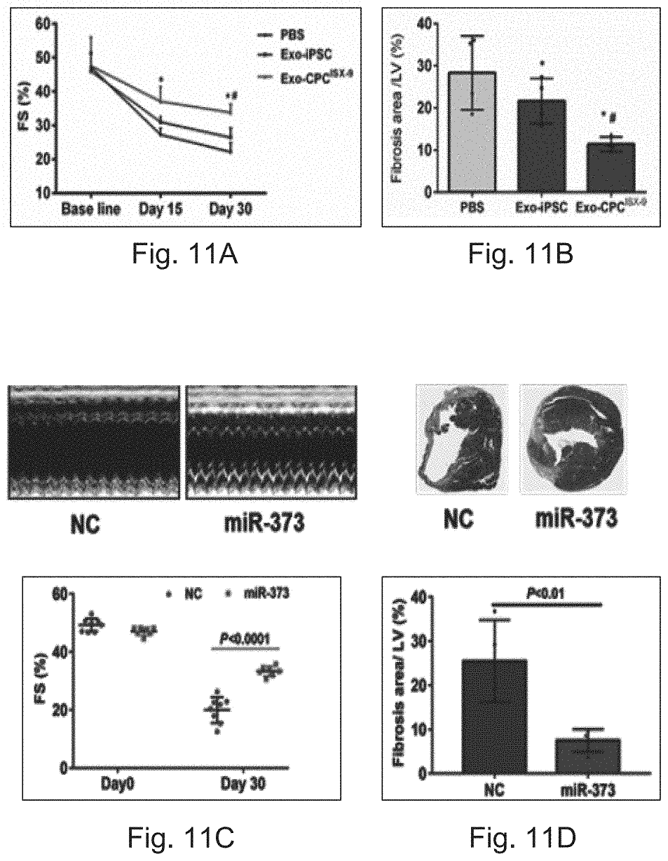

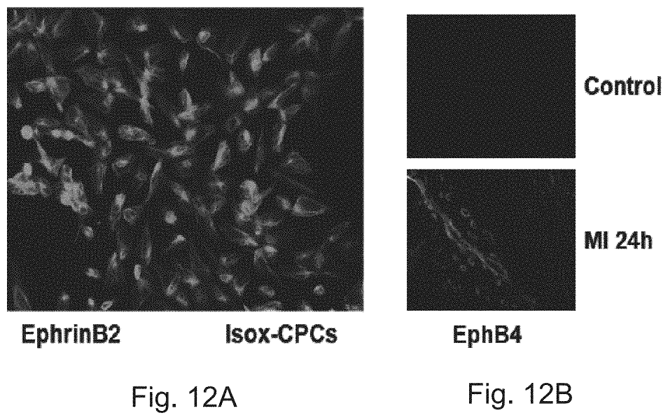

Efforts have successfully generated induced cardiac progenitor cells (iCPCs) & induced muscle progenitor cells (iMPCs) from human induced pluripotent stem cells (iPSCs) using a cardiogenic small molecule ISX-9 and other isoxazole based compounds such as Danazol & other non isoxazole based compounds such as Givinostat(FIGS. 8, 20, 21). Efforts have demonstrated that these iCPCs were strongly resistant to oxidative stress in ischemic environment (FIG. 8) and showed strong engraftment, growth and long-term improvement on cardiac function after transplantation in the infarcted mouse heart after three months (FIG. 9). The transfer of the antioxidative, proliferation and regenerative properties of Isoxazole into the exosomes of ISX-9-CPCs may be important to successful propagation of ISX-9-CPCs in the infarcted area. Therefore, exosomes secreted by these CPCs (FIG. 5) also exhibit strong cardioprotective effects in damaged heart. Work included testing whether ISX-9-CPCs had any effects on myocyte proliferation, fibrosis and function post MI. The data was compelling and supportive of our hypotheses (FIG. 10-11). miRNA-373, one of the enriched miRNAs, showed strong anti-fibrosis effects in vitro and in vivo and ultimately improved cardiac function. It was proposed that the exosomes and the corresponding miRNAs are expected to provide high levels of pleiotropic effects to reduce fibrosis and promote regeneration in the scar area.

Efforts included studying the in vivo fate of transplanted CPCs in murine heart model and; evaluating the therapeutic efficacy of exosomes together with their parent CPCs on ischemic injury and fibrosis in the infarcted heart.

MI was created in NOD/SCID mice by ligating left anterior descending coronary artery. Post-engraftment tracking of the transplanted cells and determination of their fate, the cells were labeled with PKH26 (Sigma, Product #PKH26-GL) according to manufacturer's instructions as described earlier[83-85]. For cell transplantation and their fate determination, mice were randomized in groups (n=8 each): 1) control DMEM medium; 2) hiPSCs; 3) non ISX-9-CPCs or commercial CPCs; 4) ISX-9-CPCs; 5) non ISX-9-CPCs plus their exosomes 6) ISX-9-CPCs plus their Ex; 7) 8) ISX-9-CPCs (pretreated with GW4869 which block exosomes release from CPC); 9) Group-6=CPC-derived endothelial cells.

Cardiac function after MI by serial high-resolution two-dimensional echocardiography were also recorded.

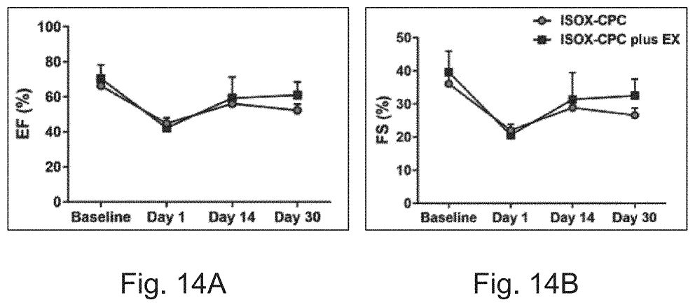

Data showed that ISX-9-CPCs expressed ephrin B2 (FIG. 12A, while inflammatory endothelial cells in infarcted hearts strongly expressed EphB4[87] and our data also showed acute infarcted heart expressed EphB4 (FIG. 12B).). We have found that EphB4/ephrinB2 signaling is involved in guiding the delivery of Exosomes to the infarcted heart leading to angiogenesis.

Exosomes were delivered by intromyocardium injection (2*10⁹ particles) and/or IV injection (2*10¹⁰ particles) after ligation; 1*10⁶ iCPCs were injected into the border zones immediately after induction of MI. Exosomes were be labeled using RNASlect Green Fluorescent cell stain to analyze exosome retention/distribution in the heart. Surviving transplanted iCPCs will be analyzed by labeling iCPCs with LuminiCell Tracker before injection. Cardiac function after MI by serial high-resolution two-dimensional echocardiography was performed.

It was expected that the overall regeneration efficiency of CPCs would be better than that of CMs because of CPC's multipotency to differentiate into CMS and vessel cells which supports our previously published strong ejection fraction improvement from our CPC's in a murine model[1].

Specific Methods used. Methods are referred to by previous publications c[1] cited herein[1,98,99]. Total RNA were isolated from cells using RNeasy Mini Kit. RT-PCR and PCR[83, 85, 95]; miRNA isolation and detection and transfection[75]; Flow cytometry[75];PCR for sry-gene[83]. Heart function[96], angiogenesis assays[95] miRNAmicroarray[83].

For post-engraftment tracking of the transplanted cells and determination of their fate, the cells were labeled with PKH26 (Sigma, Product #PKH26-GL) according to manufacturer's instructions and also previously reported[1,98].

Procedure for echocardiography in mice is described as follows. Echocardiography was performed with mice first anesthetized in an anesthesia chamber at room temperature. Induction of anesthesia is with isoflurane (4%). After the animals have been anesthetized (approximately 1 minute), they are weighed, and placed supine onto a heated (98° F.) imaging platform.

The imaging system used is HDI-5000 SONOS-CT (HP) ultrasound machine with a 7-MHz transducer. The heart was imaged in the two-dimensional mode in the parasternal long-axis and/or parasternal short-axis views which were subsequently used to position the M-mode cursor perpendicular to the ventricular septum and left ventricle posterior wall, after which M-mode images were obtained.

For each animal, measurements were obtained from 4-5 consecutive heart cycles. Measurements of ventricular septal thickness (VST), left ventricle internal dimension (LVID), and left ventricle posterior wall thickness (LVPW) were made from two-dimensionally directed M-mode images of the left ventricle in both systole and diastole. The average value from all measurements in an animal were used to determine the indices of left ventricle contractile function, i.e., left ventricle fractional shortening (LVFS) and left ventricle ejection fraction (LVEF) using the following relations LVFS=(LVEDd−LVESd)/LVEDd×100 and LVEF=[(LVEDd³−LVESd³)/LVEDd³]×100 and expressed as percentages. The scoring system we utilize is patterned after the American Society of Echocardiography's scoring system used conventionally in interpreting clinical echocardiographic studies.

### Detailed Description of Actual Data

Previously identified were highly efficient small molecules, isoxazole (ISX) and isozazole-9 (ISX-9), that are capable of transforming hiPSC into multipotent cardiac lineage cells that are highly proliferative and generate large numbers of exosomes (“EX”) containing miRNAs to elicit anti-oxidant, anti-inflammatory, immunomodulatory, pro-survival and anti-fibrotic effects. Now used is ISX-9 coupled with hypoxic “preconditioning” to generate large numbers of multipotent CPC and their EX from hiPSC for therapeutic testing in a pre-clinical animal model of myocardial infarction. Shown herein is that hiPSC pharmacologically reprogrammed into CPC and supplemented with their EX are optimally effective to regenerate infarcted myocardium.

Also shown is:

1) We have maximized the vascular and myocyte lineage differentiation by reprogramming hiPSC with ISX-9, thereby enhancing anti-oxidative, anti-inflammatory and cardiac gene promoting properties. HiPSC was differentiated into multipotent CPC with ISX-9.

2) A novel, cell free therapeutic approach utilizing CPC exosomes for promoting angiomyogenesis and cytoprotection of ischemic heart was developed. These CPC derived EXosomes actually protect CPC, cardiac myocytes and endothelial cells against ischemia.

Cell Culture:

Human iPSC cell line (ACS-1021, ATCC, USA) was maintained in mTeSR1 media (Stem Cell Technology) on vitronectin coated six-well plates with daily medium changes. CPCs were differentiated in RPMI/B27 minus insulin supplemented with ISX-9 (20 μM, dissolved in DMSO, Stem Cell Technology) for 7 days. Embryoid bodies (EB) were generated using the hanging drop method in RPMI/B27 minus insulin medium. Commercial human CPCs derived from human iPS cells (Catalog: R1093, Cellular Dynamics International) were maintained in serum-free William's E Medium supplemented with Cocktail B (CM400, Life Technologies).

Isolation of Exosomes:

Human iPSC cell line ACS-1021 (ATCC, USA), and CPCs induced by ISX-9 were cultured as described(15). In some cases, EB and commercial human CPCs were also cultured. Conditioned media was collected and exosomes were isolated by centrifugation at 3000 rpm for 30 min to remove cells and debris, followed by filtration through a 0.22 μm filter to remove the remaining debris. Then the medium was further concentrated to 500 μl using Amicon Ultra-15 100 kDa centrifugal filter units (Millipore). Isolation of exosomes in the concentrated medium was carried out through qEV size exclusion columns (Izon Science). Exosome fractions were collected and concentrated by Amicon Ultra-4 10 KDa centrifugal filter units to a final volume of <100 μl. The purified exosomes were stored at −80° C. and subsequently characterized by particle size, exosome markers and electron microscopy.

Particle Size and Concentration:

Particle size and concentration distribution were performed using tunable resistive pulse sensing (TRPS) technique with a qNano instrument (Izon Science). Briefly, the number of particles were counted (at least 600 to 1000 events) using 20 mbar pressure and NP200 nanopore membranes stretched between 46.5-47.5 mm. Calibration was performed using known concentration of beads CPC200 (diameter: 210 nm). Data were processed using Izon Control Suite software.

Transmission Electron Microscopy:

Exosome pellets were fixed with 4% paraformaldehyde (PFA). Following a total of 8 washes using PBS, grids were contrasted with a uranyl-oxalate solution for 5 minutes, and transferred to methyl-cellulose-uranyl acetate for 10 minutes on ice as previously described(16). Samples were examined on a JEOL JEM-1220 transmission electron microscope (TEM) (JEOL USA, Inc.)

Exosome Uptake by Fibroblasts:

To track exosome uptake by cultured fibroblasts, purified exosomes were labeled with PKH26, a red membrane dye (Sigma-Aldrich), according to the manufacturer's protocol. Briefly, 300 μl of exosomes was suspended into 100 μl of Diluent C, which was mixed with 1.4 μl of PKH26 dye. The labeling reaction was stopped by adding an equal volume of exosome-free FBS. Exosomes were pelleted using an exosomes column. The cultured fibroblasts in the slide chamber were incubated with labeled exosomes at 37° C. for 24 h. After incubation, cells were stained with Calcein AM (5 μM). Cells were fixed with 2% formaldehyde for 5 min and mounted with DAPI containing prolong Gold Antifade medium (Thermo Fisher Scientific). Images were taken with FV1000 confocal microscope (Olympus, Japan).

Cell Transfection and In Vitro Fibrosis Assay:

Experiments were performed using CPC^{ISX-9} grown in RPMI/B27 minus insulin, 25 nM miRNA-373 mimic, anti-miRNA-373, negative controls and RNAiMAX (Invitrogen) according to the manufacturer's instructions. miRNA-373 mimic and anti-miRNA-373 (inhibitor) were synthesized by Ambion (Life Technologies). The sequence depicted as SEQ ID NO:13 of miRNA-373 inhibitor was as follows: Anti-miRNA-373, 5′-ACACCCCAAAAUCGAAGCACUUC-3′, miRNA mimic negative control (#4464066, Ambion) and miRNA inhibitor negative control (#4464076, Ambion) were obtained from Life Technologies company. After 24 hour transfection, cells remained in culture for 24 hours and exosomes from different cell groups were collected for experimentation. The transfection efficiency was analyzed using real-time PCR. In order to test anti-fibrotic potential of exosomal miRNA-373 from CPC^{ISX-9}, fibroblasts were co-cultured with exosomes (1*10⁸/ml) from anti-miR373 inhibitor treated CPC^{ISX-9} or negative control treated CPC^{ISX-9} or miRNA-373 mimic for 48 h, and then fibroblasts were grown in serum free DMEM medium with or without TGF-β (10 ng/ml, R&D) for 48 h. Expression of pro-fibrotic genes was analyzed by real-time PCR. For the hypoxia assay, lung fibroblasts and dermal fibroblasts in culture were randomly divided into five groups and treated with: miRNA-NC, anti-miR, miRNA-373 mimic, Exo-CPC^{ISX-9} and Exo-CPC^{ISX-9}+anti-miRNA-373. After 24 hours of different pretreatments, cells were subjected to 1% O₂ in hypoxic chamber (INVIVO₂500) for 72 hours. Then, cells were fixed with 4% formaldehyde for 10 mins, and stained with α-SMA (ab5694, abcam, 1:200). Signals were visualized with Alexa Fluor 488 secondary antibodies (Life Technologies).

miRNA Array Analysis:

The NanoString nCounter Human v3 miRNA Expression Assay was used to perform the microRNA profiling analysis.

miRNA Target Gene Prediction, Gene Ontology(GO) Analysis and Luciferase Activity Assay:

miRNA target genes prediction and gene ontology analysis were carried out using DIANA mR-microT and mirPath software.

Myocardial Infarction Model:

Animal experiments were carried out both at University of Illinois at Chicago and Augusta University according to experimental protocols approved by the University of Illinois at Chicago and Augusta University Animal Care and Use Committee, and the methods were performed in accordance with the guide for the Care and Use of Laboratory Animals by the Institute of Animal Resources. MI model was generated as previously described(15). Briefly, MI was induced in 8-9-week-old NOD/SCID mice (The Jackson Laboratory) or C57/B6 mice which were anaesthetized with 2% isoflurane (isoflurane USP, HENRY SCHEIN), intubated and ventilated. The left anterior descending coronary artery (LAD) was permanently ligated with a prolene #8-0 suture. 10 mins after LAD ligation, exosomes (1*10¹²/ml) from hiPSC or CPC^{ISX-9} were injected into the myocardium along the border zone with a total of 20 μl. The same volume of PBS was injected in the control group. miRNA-373 mimic in vivo transfection was performed in C57/B6 mice. In vivo-jetPEI™ system (POLYPLUS TRANSFECTION SA) was used for intracardiac miRNA delivery. 200 pMoles miRNA-373 mimic complexed with in vivo-jetPEI at a N/P ratio of 7 in a volume of 20 μl were injected into the myocardium along the border zone immediately after LAD ligation.

Echocardiography:

Echocardiography was performed in mice anesthetized mildly with inhaled isoflurane (0.5%) using Philips iE33 ultrasound machine, equipped with L15-7io probe as described previously(15).

Histology:

Histological analysis was performed in randomly selected hearts from mice subjected to MI and treatment groups (PBS, Exo-hiPSC or Exo-CPC^{ISX-9} (n=3 per group). Mice were sacrificed after 1 month of treatment with exosomes.

Western Blot:

Exosomes and cell extracts were lysed with radio immunoprecipitation assay (RIPA) buffer supplemented with Complete Protease Inhibitor Mixture tablets (Roche Diagnostics). Protein concentration was determined by the Pierce™ BCA Protein Assay Kit (Thermo Scientific).

RNA Extraction and Real Time PCR:

Total RNA from exosomes was isolated using miRNeasy Micro Kit (Qiagen). Reverse transcription was performed using miScript II RT Kit (Qiagen).

Statistical Analysis:

Data are expressed as mean SD. Test for normality of data was performed. Statistical analysis of differences was compared by ANOVA with Bonferroni's correction for multiple comparisons. Comparisons between two groups were evaluated with Students t-test. A probability value of P<0.05 was considered statistically significant. Statistical analyses were performed using Graphpad Prism 6.0 (Chicago, USA).

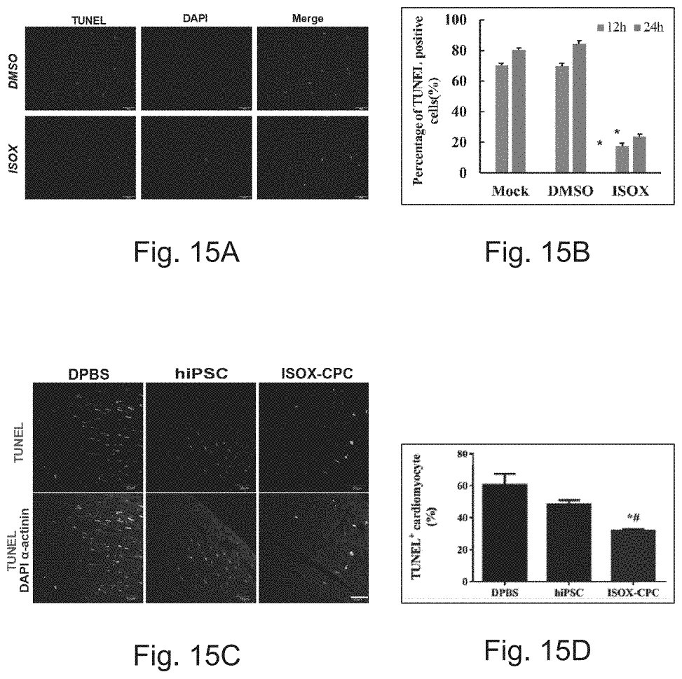

Based on the functional improvement observed with exosome treatment alone, we tested the capacity of CPC-EX to enhance cell transplantation. Indeed, the combination of ISX-9-CPCs plus EX further increased LVEF and LVFS, with continued improvement observed at 30 days post MI (FIG. 13).

The combined administration of CPC with their EX adjunctively enhanced CPC survival and engraftment in the ischemic myocardium (FIG. 13).

Moreover, ISX-9-CPC exhibited strong protection against apoptosis both in in vivo and in vitro conditions (FIG. 1). ISX-9-CPC are highly resistant to ischemia and able to efficiently engraft and grow in the oxidative environment. ISX-9-CPC have displayed resistance to oxidative and inflammatory stress, with lower percentage of apoptotic cells.

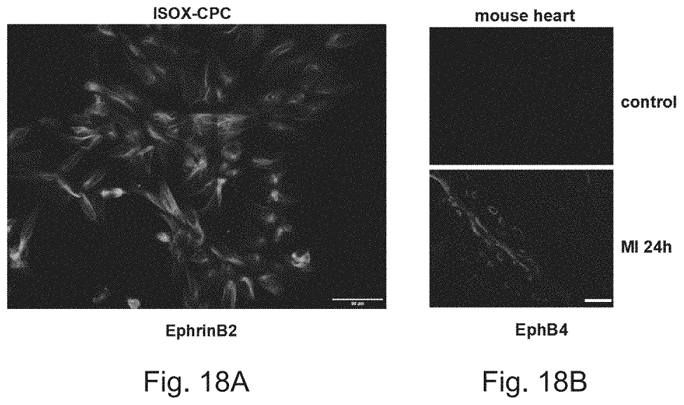

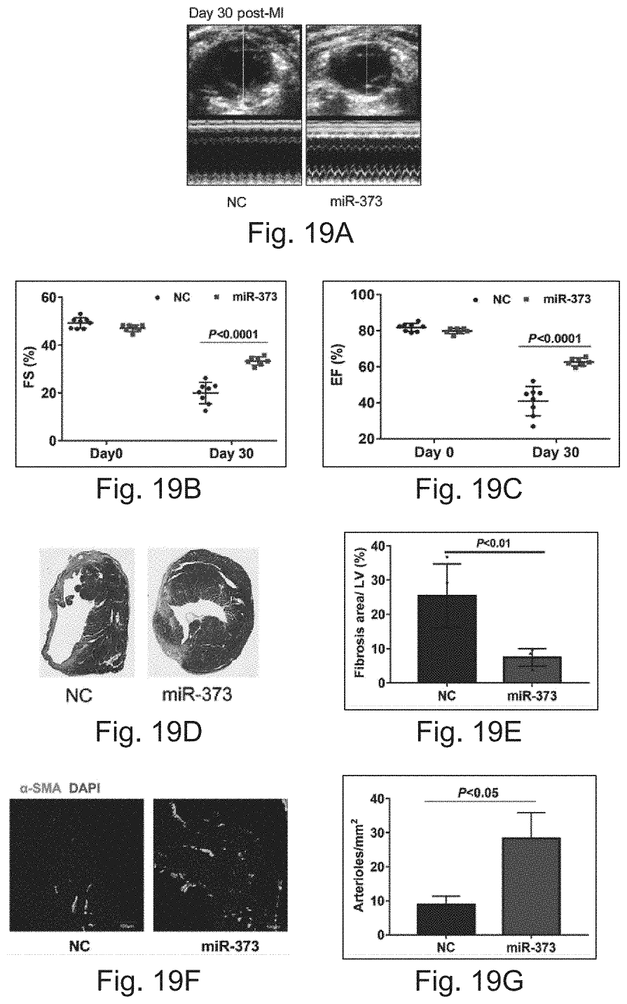

Treatment with miRNA-373 mimic, one of the miRNAs enriched in EX, prevented fibroblast stimulation by TGF-β, thereby reducing expression of fibrotic genes and their transdifferentiation into myofibroblasts(FIG. 16), while its in vivo administration promoted new vessel formation and prevented fibrosis and ultimately improved cardiac function. Under ischemic conditions it was shown that fibroblasts assume a myofibroblast-like phenotype which was surprisingly blocked by ISX-9-CPC EX and its major exosomal miRNA(miRNA-373) mimic (Fig.). Moreover, similar transformation in human aortic endothelial cells (HAEC) with TGF-β stimulation exhibiting α-SMA expression occurred (Fig.) and was also blocked by ISX-9-CPC EX and miRNA-373 mimic. FIG. 12: Treatment with miR-373 mimic, one of the miRNAs enriched in EX, prevented fibroblast stimulation by TGF-β, thereby reducing expression of fibrotic genes and their transdifferentiation into myofibroblasts, while its in vivo administration promoted new vessel formation and prevented fibrosis and ultimately improved cardiac function. It is shown here that under ischemic conditions, fibroblasts assume a myofibroblast-like phenotype which was surprisingly blocked by ISX-9-CPC EX and its major exosomal miR (miR-373) mimic (FIG. 12). Moreover, similar transformation in human aortic endothelial cells (HAEC) with TGF-β stimulation exhibiting α-SMA expression occurred (FIG. 12) and was also blocked by ISX-9-CPC EX and miR-373 mimic. We have identified ROCK-2 as a target gene of miRNA-373 using luciferase reporter assay (FIG. 17B). Thus, exosomal miRNA-373 inhibits myofibroblast transdifferentiation and EndMT elicited by TGF-β signaling via targeting the Rho/ROCK pathway.

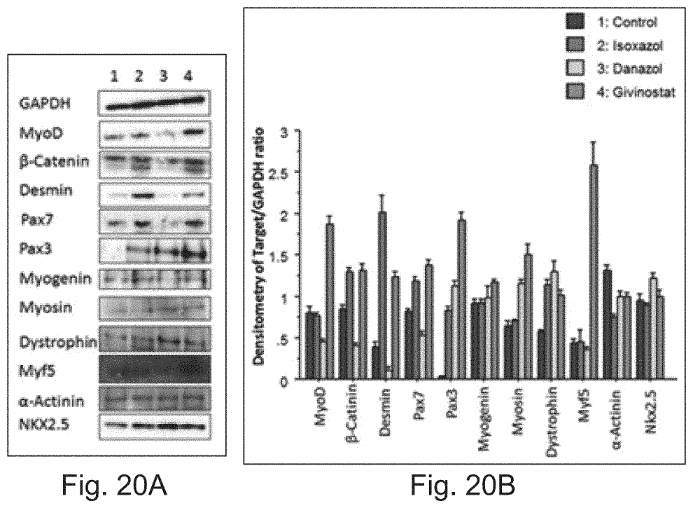

Novel data visualizing surface marker localization by immunohistology suggest that ISX-9-CPC are strongly positive for EphrinB2 (FIG. 18). EphrinB2 and EphB4 belong to a large family of cell surface receptor tyrosine kinases (RTKs) signaling molecules. EphB4/EphrinB2 signaling was reported to be involved in early stage of cardiac lineage development [78], cell migration [79] and angiogenesis [80]. Data show that ISX-9-CPC express EphrinB2 (FIG. 18A), while activated endothelial cells in infarcted hearts strongly express EphB4 (FIG. 18B).].

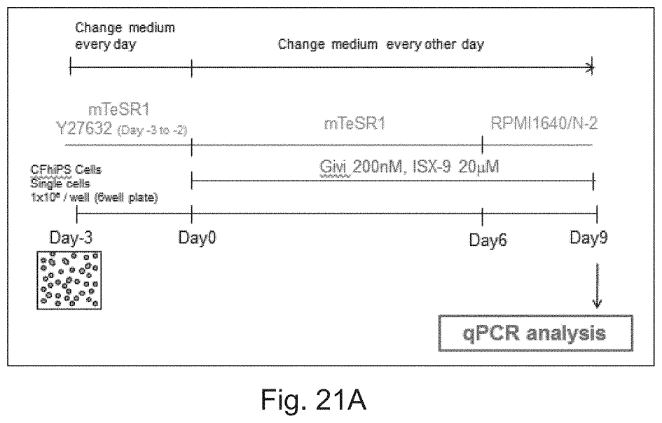

FIG. 19 miRNA-373 mimic improved cardiac function and angiogenesis and attenuated cardiac fibrosis after MI. (A) Representative M mode echocardiography images from miRNA mimic negative control (NC) treated mice and miRNA-373 mimic treated mice 30 days post-MI. FS (B) and EF (C) are shown; (P<0.001), n=8 in NC group and n=7 in miRNA-373 mimic group. (D) Representative Masson's trichrome-stained sections of hearts from NC treated mice and miRNA-373 mimic mice. (E) Quantitative analysis of fibrosis post MI. (F) Vessel density was assessed by α-SMA positive staining (green) of vascular structures. Bar=100 μm. (G) Quantitative estimate of arteriole density. P<0.05. n=3 in each group

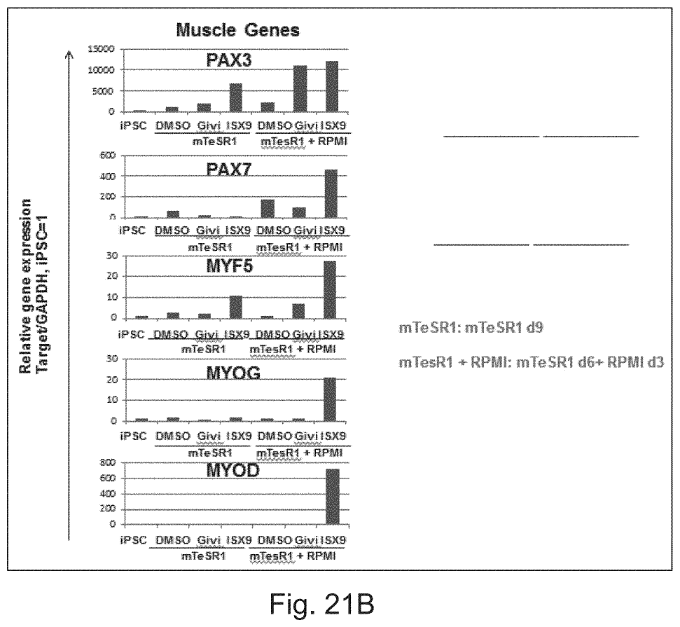

FIG. 20 demonstrates an effect of three small molecules (ISX-9, Danzol, Givinostat) on expression of cardiac and skeletal muscle genes. Real time PCR on dystrophin on small molecule (ISX9, GIV) expression in IPS cells.

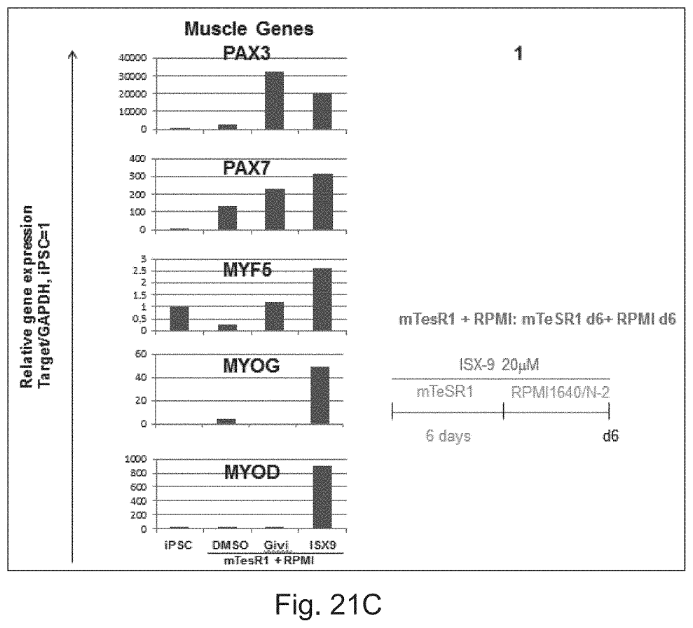

FIG. 21 demonstrates that cardiac fibrobrast derived human iPS cell colonies were dissociated with accutase and plated in the presence of Y27632. FIG. 21A schematically shows cell culture conditions for the generation of cardiac fibroblasts from human iPSCs. Briefly, to generate muscle progenitor cells (MPCs) from hiPSC in vitro, human Induced Pluripotent Stem (iPS) Cells (ATCC® ACS-1021™) induced from human cardiac fibroblasts were cultured with mTeSR™1 (STEMCELL Technologies Inc.) on Vitronectin XF (STEMCELL Technologies Inc.) coated 6-well plates. iPS Cells were passaged every 4 to 6 days with ReLeSR™ (STEMCELL Technologies Inc.). For differentiation of iPS Cells into MPCs, iPS Cells were dissociated into single cells with ACCUTASE™ (STEMCELL Technologies Inc.) into single cells and seeded at 1×10⁵ cells/cm² with mTeSR™1 supplemented with 5 μM RHO/ROCK pathway inhibitor (Y-27632, STEMCELL Technologies Inc.). After 24 hr, the medium was changed to fresh mTeSR™1. mTeSR™1 was refreshed daily during first 3 days. After 3 days, culture medium was changed to mTeSR™1 supplemented with 20 μM ISX-9 (MedChemExpress). The medium was refreshed every other day. After 6 days, the medium was switched to RPMI 1640 Medium (Thermo Fisher Scientific) supplemented with N-2 Supplement (Thermo Fisher Scientific) and 20 μM ISX-9 and refreshed every other day for another 3 to 6 days. Small molecules (Isx9 & GIV) were applied to initiate differentiation and analysed at day 9. FIG. 21B shows relative skeletal muscle gene expression by the treatment of Isx9 & Giv. FIGS. 21C and 21D show the muscle genes (PAX3, PAX7, MYF5, MYOG, MYOD), overexpression superiority in particular of ISX-9.

The following publication is fully incorporated into the present disclosure.
miRNAs in Extracellular Vesicles from iPS Derived Cardiac Progenitor Cells Effectively Reduce Fibrosis and Promote Angiogenesis in Infarcted Heart
Wanling Xuan¹, Lei Wang², Meifeng Xu³, Neal L. Weintraub¹, Muhammad Ashraf¹*Vascular Biology Center, Medical College of Georgia at Augusta University, Augusta, Ga., USA *Correspond to Prof Muhammad Ashraf, Email: mashraf@augusta.edu
Department of Pharmacology, University of Illinois at Chicago College of Medicine, Chicago, Ill., USA
Department of Pathology and Laboratory Medicine, University of Cincinnati Medical Center, Cincinnati, Ohio, USA.
Short title: Extracellular vesicles microRNAs in prevention of cardiac fibrosis

Abstract

Cardiac stem cell therapy offers the potential to ameliorate post-infarction remodeling and development of heart failure but requires optimization of cell-based approaches. Cardiac progenitor cell (CPC) induction by ISX-9, a small molecule possessing antioxidant, prosurvival and regenerative properties, represents an attractive potential approach for cell-based cardiac regenerative therapy. Here, we report that extracellular vesicles (EV) secreted by ISX-9-induced CPCs (EV-CPC^{ISX-9}) faithfully recapitulate the beneficial effects of their parent CPCs with regard to post-infarction remodeling. These EV contain a distinct repertoire of biologically-active miRNAs that promoted angiogenesis and proliferation of cardiomyocytes while ameliorating fibrosis in the infarcted heart. Amongst the highly enriched miRNAs, miR-373 was strongly antifibrotic, targeting 2 key fibrogenic genes, GDF-11 and ROCK-2. miR-373 mimic itself was highly efficacious in preventing scar formation in the infarcted myocardium. Together, these novel findings have important implications with regard to prevention of post-infarction remodeling.

Keywords: cardiac progenitor cells; extracellular vesicles; microRNAs, miR-373, fibrosis, functional recovery

Introduction

Myocardial infarction (MI) and subsequent heart failure is a leading cause of death worldwide (1). Despite advances in medical and device therapies, heart failure continues to be associated with a 5-year mortality of 50%. Stem cell therapy thus offers a great potential for cardiac tissue repair and regeneration, which might ultimately improve symptoms and longevity (2).

Notably, the beneficial effects of cardiac stem cell therapy are largely attributed to a paracrine mechanism of action that involves the release of cellular factors from the transplanted stem cells (3-5). More recent studies show that these factors are packed into small membrane bound vesicles known as extracellular vesicles (EV, 30-200 nm), which can invoke a multitude of signals (6,7). The EV contents vary amongst stem cells. Cardiac progenitor cells (CPCs) are of particular interest due to their inherent properties of cell protection, cell development, differentiation, and desirable effects imparted into the host tissue (8-10). EV from newborns improved ventricular remodeling post-MI significantly more than those derived from aging patients (11). Similarly, EV secreted from young cardiosphere-derived cells exerted stronger anti-senescent effects than those derived from aged animals (12).

Recent studies demonstrated that effects of CPCs on cardiac repair and regeneration can be faithfully recapitulated by their EV (6,13). Multiple miRNAs in EV act as mediators of cell-cell communication within the cardiovascular system (2) and can be transferred into recipient cells to regulate gene expression, thus leading to cardioprotection (11,13,14). We reported that a small molecule, ISX-9, could render CPCs (CPC^{ISX-9}) highly resistant to oxidative stress, thus permitting better survival and engraftment in the infarcted myocardium (15). Development of CPC^{ISX-9} may represent a significant advance in the cardiac stem cell field, as ISX-9 treatment circumvents the need to genetically reprogram the cells in order to enhance their function. Since CPC^{ISX-9} are well positioned for therapeutic application in humans, characterizing EV secreted from these cells is not only important to provide insight into their mechanisms of action, but also may help to identify novel miRNAs involved in cardioprotection.

Since the EV cargo contents are unique to each cell type and consequently their effectiveness is variable. Considering this limitation, we have generated multipotent CPCs from human induced pluripotent stem cells (hiPSCs) using a unique small molecule with anti-oxidant and regenerative properties capable of successfully propagating in the infarcted myocardium. Since CPC are the cells of choice for regeneration, their EV would be considered to be more effective in cardiac repair than EV from non CPC. Therefore, the purpose of the study was to exploit EV from hiPSC-CPC induced with ISX-9 and not the role of ISX-9 per se on EV release from CPC. Here, we tested the hypothesis that EV secreted by ISX-9-induced CPCs (EV-CPC^{ISX-9}) will be highly efficacious in cardiac repair owing to the unique properties of their parent cells. EV-CPC^{ISX-9} exerted strong effects on fibrosis and angiogenesis in the infarcted myocardium of mice. Mechanistically, we identified miR-373 enriched in EV-CPC^{ISX-9}, which elicited strong anti-fibrotic effects by targeting two genes, growth differentiation factor 11 (GDF-11) and Rho-associated coiled-coil containing kinase-2 (ROCK-2), and showed that miR-373 mimic effectively inhibits post-infarct cardiac remodeling.

Materials and Method

Cell Culture

Human iPSC cell line (ACS-1021, ATCC, USA) was maintained in mTeSR1 media (Stem Cell Technology) on vitronectin coated six-well plates with daily medium changes. Cells were passaged with ReLeSR™ reagent every 4-7 days according to the manufacturer's protocol (Stem Cell Technology). For CPC generation, briefly, hiPSCs maintained on vitronectin coated six-well plates in mTeSR1 media (Stem Cell Technology) were dissociated into single cells using accutase (Invitrogen) at 37° C. for 10 min and then were seeded on to a vitronectin-coated six-well plates at 1×10⁶ cells/well in mTeSR1 supplemented with 5 μM ROCK inhibitor (Y-27632, Stem Cell Technology) for 24 h. The following day, cells were cultured in mTesR1 with daily medium change for 3 days. Afterwards, the medium was switched to RPMI/B27 minus insulin supplemented with ISX-9 (20 μM, dissolved in DMSO, Stem Cell Technology) for 7 days. Embryoid bodies (EB) were generated using the hanging drop method in RPMI/B27 minus insulin medium. Human dermal fibroblast cell line (CC-2511) and lung fibroblast cell line (CC-2512) were obtained from Lonza Company. Briefly, fibroblasts were maintained in FibroGRO™ Complete Media (Millipore Sigma). Cells were passaged with accutase; passages 2-4 were used for experiments. Commercial human CPCs (control-CPC) derived from human iPS cells (Catalog: R1093, Cellular Dynamics International) were maintained in serum-free William's E Medium supplemented with Cocktail B (CM400, Life Technologies). Passage 2 was used for experiments.

Isolation of EV

Human iPSC cell line ACS-1021 (ATCC, USA), and CPCs induced by ISX-9 were cultured as described(15). In some cases, EB and commercial human CPCs were also cultured. Conditioned media was collected and EV were isolated by centrifugation at 3000 rpm for 30 min to remove cells and debris, followed by filtration through a 0.22 μm filter to remove the remaining debris. Then the medium was further concentrated to 500 μl using Amicon Ultra-15 100 kDa centrifugal filter units (Millipore). Isolation of EV in the concentrated medium was carried out through qEV size exclusion columns (Izon Science). EV fractions were collected and concentrated by Amicon Ultra-4 10 KDa centrifugal filter units to a final volume of <100 μl. The purified EV were stored at −80° C. and subsequently characterized by particle size, EV markers and electron microscopy.

Particle Size and Concentration Distribution Measurement with Tunable Resistive Pulse Sensing

Particle size and concentration distribution were performed using tunable resistive pulse sensing (TRPS) technique with a qNano instrument (Izon Science). Briefly, the number of particles were counted (at least 600 to 1000 events) using 20 mbar pressure and NP200 nanopore membranes stretched between 46.5-47.5 mm. Calibration was performed using known concentration of beads CPC200 (diameter: 210 nm). Data were processed using Izon Control Suite software.

Transmission Electron Microscopy

EV pellets were fixed with 4% paraformaldehyde (PFA). Following a total of 8 washes using PBS, grids were contrasted with a uranyl-oxalate solution for 5 minutes, and transferred to methyl-cellulose-uranyl acetate for 10 minutes on ice as previously described (16). Samples were examined on a JEOL JEM-1220 transmission electron microscope (TEM) (JEOL USA, Inc.)

EV Uptake by Fibroblasts

To track EV uptake by cultured fibroblasts, purified EV were labeled with PKH26, a red membrane dye (Sigma-Aldrich), according to the manufacturer's protocol. Briefly, 300 μl of EV was suspended into 100 μl of Diluent C, which was mixed with 1.4 μl of PKH26 dye. The labeling reaction was stopped by adding an equal volume of EV-free FBS. Exosome Spin Columns (Cat. 4484449, Thermo Fisher Scientific) was used to remove unincorporated PKH26. The cultured fibroblasts in the slide chamber were incubated with labeled EV at 37° C. for 24 h. After incubation, cells were stained with Calcein AM (5 μM). Cells were fixed with 2% formaldehyde for 5 min and mounted with DAPI containing prolong Gold Antifade medium (Thermo Fisher Scientific). Images were taken with FV1000 confocal microscope (Olympus, Japan).

Cell Transfection and In Vitro Fibrosis Assay

Experiments were performed using CPC^{ISX-9} grown in RPMI/B27 minus insulin, 25 nM miR-373 mimic, anti-miR-373, negative controls and RNAiMAX (Invitrogen) according to the manufacturer's instructions. miR-373 mimic and anti-miR-373 (inhibitor) were synthesized by Ambion (Life Technologies). The sequence of miR-373 inhibitor identified as SEQ ID NO:13 was as follows: Anti-miR-373, 5′-ACACCCCAAAAUCGAAGCACUUC-3′. miRNA mimic negative control (#4464066, Ambion) and miRNA inhibitor negative control (#4464076, Ambion) were obtained from Life Technologies company. After 24 h transfection, cells remained in culture for 24 h and EV from different cell groups were collected for experimentation. The transfection efficiency was analyzed using real-time PCR. In order to test anti-fibrotic potential of miR-373 from EV-CPC^{ISX-9}, fibroblasts were co-cultured with EV (1*10⁸/ml) from anti-miR373 inhibitor treated CPC^{ISX-9} or negative control treated CPC^{ISX-9} or miR-373 mimic for 48 h, and then fibroblasts were grown in serum free DMEM medium with or without TGF-β (10 ng/ml, R&D) for 48 h. Expression of pro-fibrotic genes was analyzed by real-time PCR. For the hypoxia assay, lung fibroblasts and dermal fibroblasts in culture were randomly divided into five groups and treated with: miR-NC, anti-miR, miR-373 mimic, EV-CPC^{ISX-9} and EV-CPC^{ISX-9}-9+anti-miR-373. After 24 h of different pretreatments, cells were subjected to 1% O₂ in hypoxic chamber (INVIVO₂ 500) for 72 h. Then, cells were fixed with 4% formaldehyde for 10 mins, and stained with α-SMA (ab5694, abeam, 1:200). Signals were visualized with Alexa Fluor 488 secondary antibodies (Life Technologies).

miRNA Array Analysis

The NanoString nCounter Human v3 miRNA Expression Assay was used to perform the microRNA profiling analysis. The assay allows measurement of 800 different microRNAs at the same time for each sample. 3.5 μl of suspension RNA was annealed with multiplexed DNA tags (miR-tag) and bridges target specifics. Mature microRNAs were bonded to specific miR-tags using a Ligase enzyme, and excess tags were removed by enzyme clean-up step. The tagged microRNA product was diluted 1 to 5, and 5 μl was combined with 20 μl of reporter probes in hybridization buffer and 5 μl of Capture probes overnight (17 hours) at 65° C. to permit hybridization of probes with specific target sequences. Excess probes were removed using two-step magnetic bead-based purification on an automated fluidic handling system (nCounter Prep Station) and target/probe complexes were immobilized on the cartridge for data collection. The nCounter Digital Analyzer took images of immobilized fluorescent reporters in the sample cartridge with a CCD camera through a microscope objective lens. For each cartridge, a high-density scan encompassing 325 fields of view was performed. NanoString raw data was analyzed with nSolver™ software, provided by NanoString Technologies. The mean plus 2 times the standard deviation of Negative Control Probes was used to perform background subtraction; positives were used to perform technical normalization to adjust lane by lane variability due to differences in hybridization, purification or binding. Data was then normalized by calculating the geometric mean of the spikes present in each sample, as recommended by NanoString. One-way ANOVA was used to calculate the P value; targets with P<0.05 were selected.

miRNA Target Gene Prediction, Gene Ontology(GO) Analysis and Luciferase Activity Assay

miRNA target genes prediction and gene ontology analysis were carried out using DIANA mR-microT and mirPath software. Differentially miRNA target genes in significant GO and pathway categories, obtained from GO and pathway analyses, were analyzed with mirPath v.3 software. GO biological process includes biological processes, molecular function and cellular component of upregulated and downregulated genes.

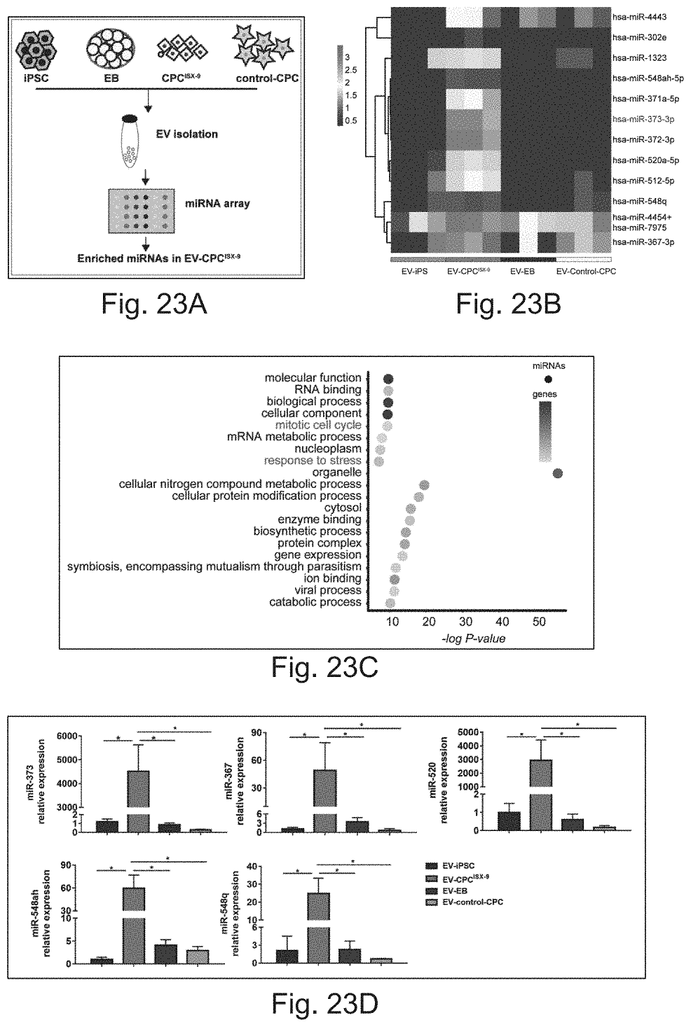

For luciferase activity assay, using standard procedures, wild-type (WT) or mutant 3′untranslated regions (UTRs) of GDF-11 or ROCK-2 were subcloned into the pLenti-UTR-Dual-Luc vector (abm, Canada) obtaining the sequence as shown in SEQ ID NO: 14 (FIG. 24C) downstream of the luciferase gene. The predicted binding sites and mutant sequences from SEQ ID NO: 15 to SEQ ID NO:20 are shown in FIG. 24D. GDF-11-3′UTR-WT (SEQ ID NO: 18), GDF-11-3′UTR-Mut (SEQ ID NO: 20), ROCK2-3′UTR-WT (SEQ ID NO: 15), or ROCK2-3′UTR-Mut (SEQ ID NO:17) vectors were co-transfected with miR-373 mimic or negative control into 293FT cells using Lipofectamine 3000 for 48 h. Transfected cells were analyzed using the dual-luciferase reporter assay system (Promega). The luciferase activity was normalized using Renilla activity. Myocardial infarction model

Animal experiments were carried out both at University of Illinois at Chicago and Augusta University according to experimental protocols approved by the University of Illinois at Chicago and Augusta University Animal Care and Use Committee, and the methods were performed in accordance with the guide for the Care and Use of Laboratory Animals by the Institute of Animal Resources. MI model was generated as previously described(15). Briefly, MI was induced in 8-9-week-old NOD/SCID mice (The Jackson Laboratory) or C57/B6 mice which were anaesthetized with 2% isoflurane (isoflurane USP, HENRY SCHEIN), intubated and ventilated. The left anterior descending coronary artery (LAD) was permanently ligated with a prolene #8-0 suture. 10 mins after LAD ligation, EV (1*10¹²/ml) from hiPSC or CPC^{ISX-9} were injected into the myocardium along the border zone with a total of 20 μl. The same volume of PBS was injected in the control group. miR-373 mimic in vivo transfection was performed in C57/B6 mice. In vivo-jetPEI™ system (POLYPLUS TRANSFECTION SA) was used for intracardiac miRNA delivery. 200 pMoles miR-373 mimic complexed with in vivo-jetPEI at a N/P ratio of 7 in a volume of 20 μl were injected into the myocardium along the border zone approximately 10 mins after LAD ligation.

Echocardiography

Echocardiography was performed in mice anesthetized mildly with inhaled isoflurane (0.5%) using Philips iE33 ultrasound machine, equipped with L15-7io probe as described previously(15). Hearts were imaged in 2D in the parasternal long-axis and/or parasternal short-axis views at the level of the highest LV diameter. Measurements of left ventricular end diastolic diameter (LVDd), and left ventricular end systolic diameter (LVDs) were made from 2D M-mode images of the left ventricle in both systole and diastole. Left ventricle fractional shortening (LVFS) was calculated using the following formula: LVFS=(LVDd-LVDs)/LVDd×100. Ejection fraction (EF), Left ventricular end diastolic volume (LVEDv) and left ventricular end systolic volume (LVESv) were calculated using the following formula: 7.0×LVEDd/(2.4+LVDd) and 7.0×LVESd/(2.4+LVDs) respectively; left ventricular ejection fraction (LVEF) was calculated as (LVEDv−LVESv)/LVEDv×100%. LVFS and EF were expressed as percentages.

Histology

Histological analysis was performed in randomly selected hearts from mice subjected to MI and treatment groups (PBS, EV-hiPSC or EV-CPC^{ISX-9} (n=3 per group). Mice were sacrificed after 1 month of treatment with EV. For immunostaining, hearts were fixed with 4% PFA for 1 hour at room temperature and replaced by 30% sucrose overnight at 4° C. Afterwards, hearts were cryopreserved in an optical cutting temperature (OCT) compound (Tissue Tek) at −80° C. Hearts were sliced into 5-μm-thick frozen sections and incubated with primary antibodies including α-sarcomeric actinin (A7811, Sigma, 1:200), ki67 (ab16667, abeam, 1:500), cTnT (13-11, Thermo fisher Scientific, 1:300) and SMA (ab5694, abeam, 1:300). Signals were visualized with Alexa Fluor 647 and Alexa Fluor 488 secondary antibodies (Life Technologies). Images were recorded on a confocal microscope (FV1000, Olympus, Japan). For fibrosis analysis, hearts were embedded in paraffin and cut at 5-μm-thick sections. Masson trichrome staining was performed according to the manufacturer's protocol (HT-15, Sigma). The size of LV area and scar area were measured using the ImageJ software. 4 sections (EV treated mice) and 6 sections (miR-373 mimic treated mice) were analysed per heart. The fibrosis area was determined as the ratio of scar area to LV area and expressed as percentage. Vessel density was assessed in 9 animals (3 in each group) in NOD/SCID mice, and 6 animals in C57/B6 mice (3 in each group) which were sacrificed at 1M after MI. The number of vessels was blindly counted on 27 sections (3 sections per heart) in NOD/SCID mice or 18 sections (3 sections per heart) in C57/B6 mice in the infarct and border areas of all mice after staining with an antibody α-SMA using a fluorescence microscope at a 400× magnification. Vascular density was determined by counting α-SMA positive vascular structures. The number of vessels in each section was averaged and expressed as the number of vessels per field (0.2 mm²)

Western Blot

EV and cell extracts were lysed with radio immunoprecipitation assay (RIPA) buffer supplemented with Complete Protease Inhibitor Mixture tablets (Roche Diagnostics). Protein concentration was determined by the Pierce™ BCA Protein Assay Kit (Thermo Scientific). 10 μg proteins were separated by SDS/PAGE and transferred to PVDF membrane (BioRad). Membranes were incubated with primary antibodies against the following proteins overnight at 4° C.: mouse anti-tsg101 (sc-365062, Santa Cruz), mouse anti-Calnexin (sc-23954, Santa Cruz), goat-anti-Hsp70 (EXOAB-Hsp70A-1, SBI), rabbit anti-CD9 (#13174, CST), rabbit anti-Flotillin-1(#18634, CST), mouse anti-GADPH (sc-365062, Santa Cruz). The membrane was then washed, incubated with an anti-mouse/rabbit/goat peroxidase-conjugated secondary antibody. Immunoreactive bands were visualized by the enhanced chemiluminescence method (Pierce, Thermo Scientific) with a western blotting detection system (Fluorchem E, ProteinSimple USA) and were quantified by densitometry with ImageJ software.

RNA Extraction and Real Time PCR

Total RNA from EV was isolated using miRNeasy Micro Kit (Qiagen). Reverse transcription was performed using miScript II RT Kit (Qiagen). Quantification of mRNA and selected miRNAs were performed by real-time system quantstudio3 (ABI) using miScript SYBR Green PCR Kit (Qiagen). miRNA primer sequences are shown in Table S1, and mRNA primer sequences are shown in Table S2. Expression levels of selected miRNAs were quantified, validated with RT-PCR and values are expressed as 2^{-ΔΔCT} with respect to the expression of the reference U6. The primer of U6 was provided in the PCR kit.

Statistical Analysis

Data are expressed as mean SD. Test for normality of data was performed. Statistical analysis of differences was compared by ANOVA with Bonferroni's correction for multiple comparisons. Comparisons between two groups were evaluated with Students t-test. A probability value of P<0.05 was considered statistically significant. Statistical analyses were performed using Graphpad Prism 6.0 (Chicago, USA).

Results

Characterization of EV Secreted by ISX-9 Induced Cardiac Progenitors

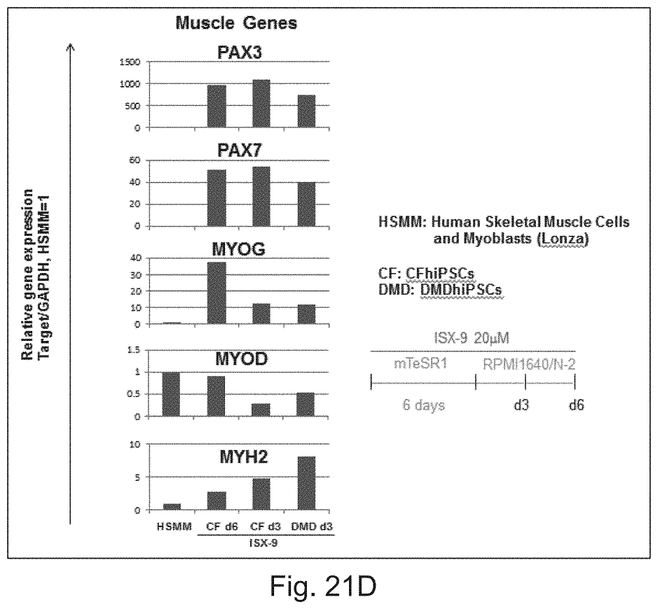

Electron microscopy analysis showed that secreted EV measured 160-170 nm in diameter (FIGS. 22A, 22B and 22D). No significant difference in size was observed amongst the groups (FIG. 1D, IE). Additionally, EV were enriched in EV-specific markers Tsg101, CD9, Hsp70 and Flotillin-1. Calnexin was absent in isolated EV (FIG. 22C), confirming their purity.

EV-CPC^{ISX-9} Exhibit a Unique miRNA Profile

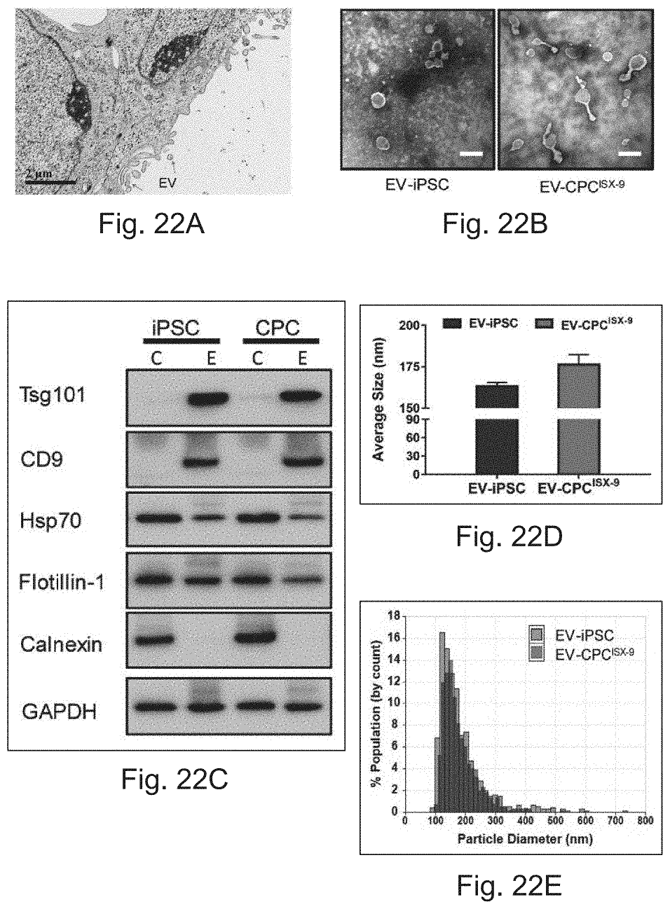

Next, we performed miRNA array to determine whether miRNA cargo content of EV-CPC^{ISX-9} differs from that of hiPSCs, EBs and commercial CPCs (FIG. 23A). Global miRNA profiling showed that EV-CPC^{ISX-9} had a unique miRNA expression signature very different from that of the other derived EV. miR-520/-373 family members, including miR-371, miR-302, miR-372, miR-373 and miR-520, as well as miR-512, miR-548 and miR-367, were significantly upregulated in EV-CPC^{ISX-9} compared to EV from other parent cells (FIG. 23B) (GEO number is pending). Furthermore, the expression of these enriched miRNAs (miR-373/miR-548/miR-367/miR-520) was validated with real-time PCR (FIG. 23D). The target genes of the differentially expressed miRNAs control a broad range of biological functions. Biological process of gene ontology (GO) enrichment analysis based on enriched miRNA-targeted genes demonstrated that some target genes were significantly enriched in responses to stress and cell cycle (FIG. 23C).

Anti-Fibrotic Effects Mediated by miR-373 Derived from EV-CPC^{ISX-9}

We hypothesized that the enriched miR-373 EV from CPC^{ISX-9} exert anti-fibrotic effects. First, using PKH26 labeling, we confirmed that EV were internalized by fibroblasts and localized in the perinuclear region. miR-373 expression level was markedly higher in EV than in their donor cells, CPC^{ISX-9}. Inhibition of miR-373 in CPC^{ISX-9} resulted in decreased miR-373 expression in EV-CPC^{ISX-9} and reduced miR-373 expression in fibroblasts incubated with these EV compared to those from control cells. Stimulation of fibroblasts with TGF-β led to significant upregulation of fibrotic genes (MMP-2, TIMP-2, TIMP-1, FN1, CTGF and MMP-9). Upon pretreatment of fibroblasts with EV from control CPC^{ISX-9}, upregulation of these fibrotic genes by TGF-β was inhibited. However, inhibition of miR-373 in CPC^{ISX-9} abrogated the capacity of the EV to inhibit fibrotic gene expression. Conversely, pretreatment of fibroblasts with miR-373 mimic inhibited TGF-β induced expression of fibrotic genes (FIG. 24A). We also performed experiments in a second cellular model of fibrosis by exposing the fibroblasts to hypoxia. FIG. 24B shows that with 72 h exposure in a hypoxic environment, both lung and dermal fibroblasts differentiated into myofibroblasts expressing α-SMA. As expected, miR-373 mimic significantly inhibited fibroblast transdifferentiation into myofibroblasts under hypoxia. Similarly, fibroblasts failed to transdifferentiate into myofibroblasts when they were pretreated with EV from CPC^{ISX-9}. Taken together, these results suggest that miR-373 contained in EV-CPC^{ISX-9} suppresses fibrosis both in in vitro and in vivo levels.

Although a previous study reported miR-373 might target TGF-β(17), here we identified two new potential target genes of miR-373, GDF-11 and ROCK-2, using DIANA mR-microT software and dual-luciferase reporter assay. The 3′-UTR binding sites are shown in FIG. 3D. Next the predicted binding sites of GDF-11 and ROCK-2 were cloned into 3′-UTR of the dual luciferase vector respectively (FIG. 24C) and transiently transfected into 293FT cells. miR-373 mimic transfection significantly decreased the relative luciferase activity when co-transfected with GDF-11 and ROCK-2 3′-UTR vectors. When 3′-UTR binding sites were mutated, the repression of GDF-11 and ROCK-2 3′-UTR by miR-373 mimic was attenuated (FIG. 24E). Notably, the human miR-373 3′ UTR binding sites for GDF-11 and ROCK-2 are conserved among several species, including mice and rats.

Moreover, under hypoxic conditions, the expression of GDF-11 and ROCK-2 was increased in lung fibroblasts (FIG. 24F), while pretreatment with miR-373 mimic or EV-CPC^{ISX-9} significantly inhibited expression of these two genes, supporting the contention that miR-373 targets GDF-11 and ROCK-2 (FIG. 24).

EV-CPC^{ISX-9} Promoted CM Proliferation and Angiogenesis, and Reversed Ventricular Remodeling, in Mice Post MI

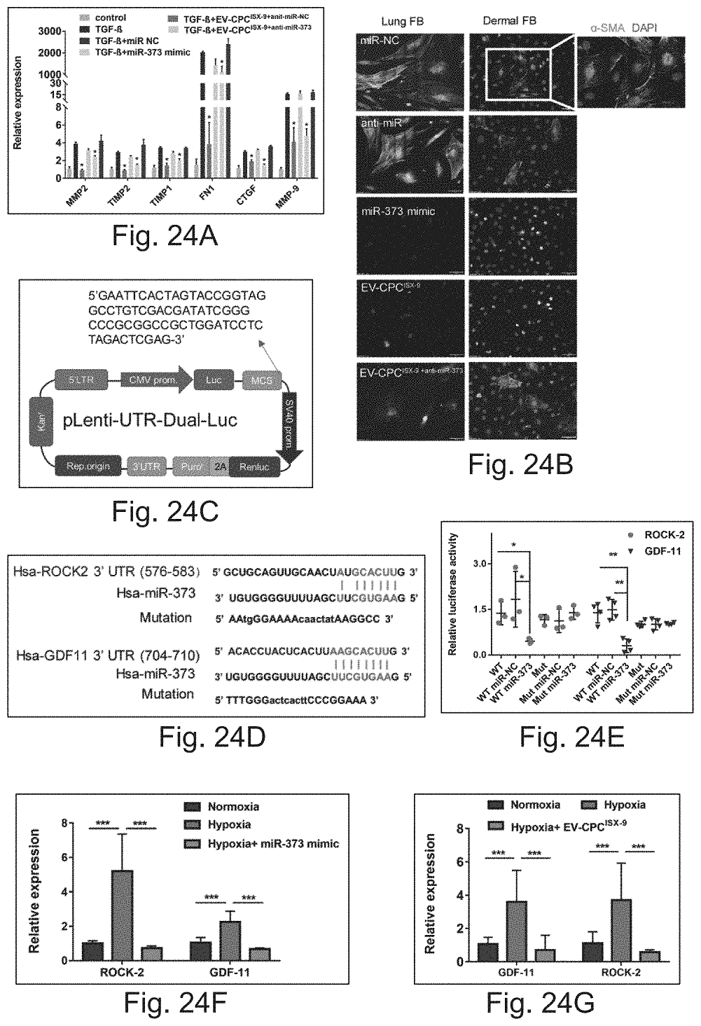

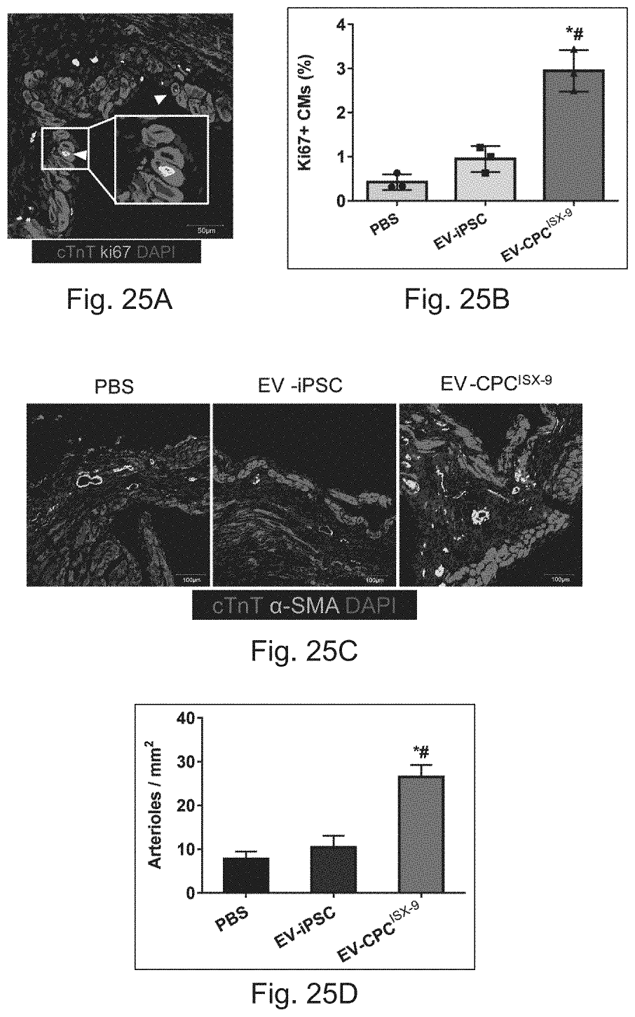

Next, we determined the effects of treatment with EV-CPC^{ISX-9} in a mouse model of MI. compared to PBS and EV-hiPSCs, EV-CPC^{ISX-9} treatment boosted cardiomyocyte proliferation in the infarcted hearts. FIG. 25A & 25B show representative images and quantitative data of proliferating Ki67 and α-actinin positive cardiomyocytes in the peri-infarct region. We further determined the impact of EV-CPC^{ISX-9} on angiogenesis using tube formation assay. We found that EV-CPC^{ISX-9} indeed increased average tube length of human aortic endothelia cells (HAECs) in vitro. Remarkably, EV-CPC^{ISX-9} also reduced oxidant induced changes in HAEC. Similarly, the vessel density as identified by α-SMA staining and tube like structures (FIG. 25C, 25D) in the infarcted region was also increased by treatment with EV-CPC^{ISX-9}. The deterioration in cardiac function, as noted by rise in LVEDD and LVESD as well as a progressive decline in LVFS 1-month post-MI, was attenuated by EV-CPC^{ISX-9} treatment. EV-CPC^{ISX-9} slowed the progression of left ventricle enlargement (LVDs, 2.35±0.31 mm vs. 2.79±0.30 mm and 3.047±0.35 mm; LVDd, 3.54±0.40 mm vs. 3.79±0.33 mm and 3.91±0.38 mm in EV-hiPSC, PBS and EV-CPC^{ISX-9} treated groups, respectively) and improved cardiac function (LVFS: 33.77±2.42% vs. 26.44±2.79% and 22.16±2.78% in EV-hiPSC, PBS and EV-CPC^{ISX-9} treated groups, respectively; LVEF: 70.82±3.12% vs. 52.67±4.78% and 60.05±4.58% in EV-hiPSC and PBS treated groups) (FIG. 26 A-E). Moreover, smaller scar size was observed in mice treated with EV-CPC^{ISX-9} compared with PBS and EV-hiPSC (P<0.01; FIG. 26F, 26G).

miR-373 Mimic Attenuated Cardiac Fibrosis and Improved Cardiac Function and Angiogenesis after MI

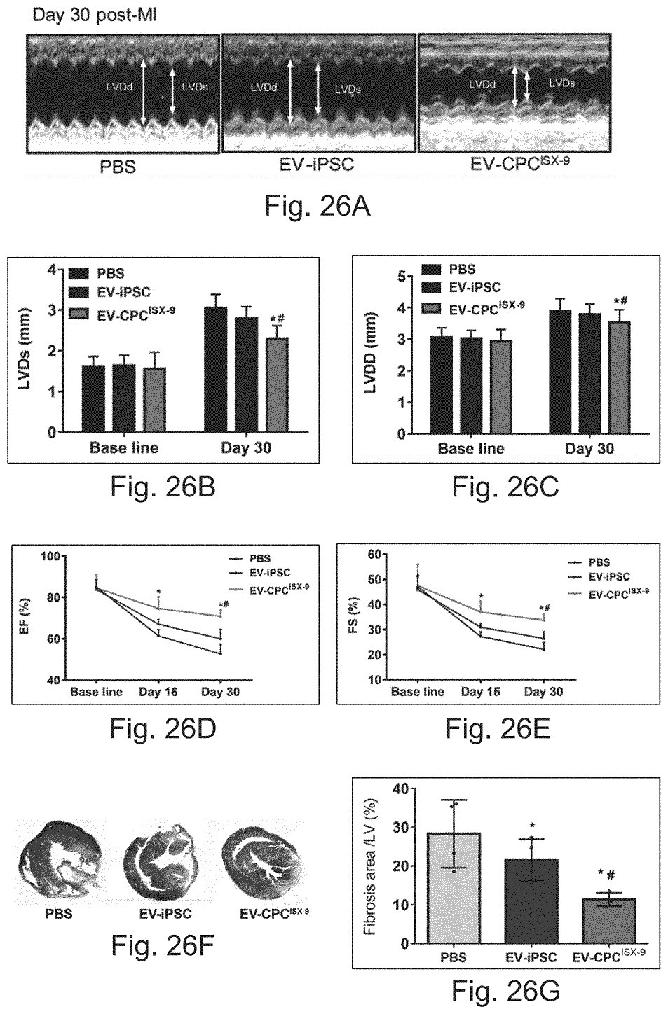

Having demonstrated the anti-fibrotic effects of miR-373 in vitro, we further explored and validated direct effects of miR-373 on post-infarct remodeling and fibrosis. We delivered miR-373 mimic by intramyocardial injection after LAD ligation. After 1 month, miR-373 mimic treatment significantly improved cardiac function compared to control mice (LVFS: 33.38±1.72% vs. 19.98±4.45% in NC treated mice; LVEF: 62.25±2.16% vs. 40.87±8.17% in NC treated mice.) FIG. 27A-C). In addition, miR-373 mimic dramatically attenuated cardiac fibrosis in comparison to NC treatment (FIG. 27D-E and). Moreover, the vessel density as identified by α-SMA staining and tube like structures (FIGS. 27F and G) in the infarcted region was increased by miR-373 treatment. Despite the effectiveness of EV-CPC^{ISX-9} or miR-373, no difference in survival rate between the two groups was observed due to death because of cardiac rupture.

Discussion

Stem cell based therapy has been well recognized to improve cardiac function following MI. While this therapy has merits, it also suffers from several limitations, particularly lack of suitable stem cell type and their insufficient engraftment and growth, ranging from no new cell formation to sparse newly formed cells in the infarcted tissue (18-20). Cellular therapy has been propelled by the invention of iPS cells, which have the ability to transform into different progenitor cells types. The cardiac progenitors derived from iPS cells and their counter parts have been used both in animal models of MI (21,22) and in humans (23) with promising results. While the underlying mechanisms of beneficial effects of stem cell therapy remain a point of debate, increasing evidence suggests that paracrine factors play a key role by reducing cell death and stimulating cell migration and proliferation (24,25). This paracrine signaling involves the secretion of small vesicles or EV harboring multiple miRNAs, proteins and other factors that mediate protection in the heart. Secreted extracellular vehicles (EVs), such as EV, are packed with potent pro-repair proteins and RNA cargo that are both cell type-specific as well as differentially produced and secreted according to the cellular environment. Additionally, miRNA profiles of EV might be distinct from cellular miRNA patterns (26).

In this study, EV derived from CPC^{ISX-9} were found to be highly cardioprotective, and the effect can in part be attributed to their specific miRNA content. CPC^{ISX-9} derived EV were highly enriched with miR-520/-373 family members including miR-371, miR-372, miR-373 and miR-520, as well as miR-512, miR-548 and miR-367, compared to EV derived from other parent cells. miR-373, which was particularly highly enriched in EV-CPC^{ISX-9}, was first identified as a human embryonic stem cell (ESC)-specific miRNA, implicated in the regulation of cell proliferation, apoptosis, senescence, migration and invasion, as well as DNA damage repair following hypoxia stress (27).

Little has been published regarding the putative role of miR-373 in regulating cardiac pathology or function. In a mouse model of type 1 diabetic cardiomyopathy, miR-373 was found to be significantly downregulated, and application of a miR-373 mimic to neonatal cardiomyocytes exposed to elevated glucose in vitro suppressed cell hypertrophy (28). Fibrosis is also an important pathological feature of diabetic cardiomyopathy, but effects of miR-373 on fibrosis were not investigated in that study. Fibrosis plays a prominent role in ventricular remodeling and ultimately in the pathogenesis of heart failure after MI. A previous study reported that miR-373 targeted the members of TGF-β signaling including TGF-β receptor2 and Smad2, and promoted mesoderm differentiation in human embryonic stem cells (17). miR-373-3p expression was low in hypertrophic myocardium with diffuse myocardial fibrosis (29), suggesting that miR-373 may function as an anti-fibrotic miRNA. Thus, we hypothesized that because of their enrichment in miR-373, EV-CPC^{ISX-9} might produce strong anti-fibrotic effects to modulate cardiac remodeling.

Our results indicate that both EV-CPC^{ISX-9} and miR-373 mimic inhibited TGF-β- and hypoxia-induced fibrotic gene expression in vitro. With inhibition of miR-373 in EV-CPC^{ISX-9}, or treatment with miR-373 inhibitor, the effects on fibrotic gene expression were abrogated. The luciferase activity assay confirmed that miR-373 targeted GDF-11 and ROCK-2, both known to be involved in fibrosis. An isoform of Rho-associated coiled-coil forming protein kinase 2, ROCK-2 is reportedly a critical mediator of organ fibrosis. Inhibition of ROCK-2 protected ROCK-2-haploinsufficient mice from bleomycin-induced myofibroblast differentiation and pulmonary fibrosis (30), while its activation was implicated in development of idiopathic pulmonary fibrosis (31). Additionally, fibroblast-specific ROCK2 was reported to promote cardiac hypertrophy, fibrosis, and diastolic dysfunction due to upregulation of profibrotic gene (CTGF) and promyofibroblast differentiation (α-SMA) genes (32). Mutant mice with elevated fibroblast ROCK activity exhibited enhanced Ang II-stimulated cardiac hypertrophy and fibrosis (32). The role of the second identified target gene, GDF-11 is more controversial. It was reported to beneficially reverse age-related cardiac hypertrophy and skeletal muscle dysfunction (33,34), while other reports suggest that it promotes cardiac and skeletal muscle dysfunction and wasting (35), inhibits skeletal muscle regeneration (36), exerts pro-fibrotic effects (37), and renal failure and interstitial fibrosis(38). Therefore, our data suggest that miR-373 inhibited profibrotic gene upregulation and myofibroblast differentiation in fibroblasts by targeting GDF-11 and ROCK-2.

In vivo data showed that compared with EV-hiPS and PBS, EV-CPC^{ISX-9} treatment reduced fibrosis and improved cardiac function, thus supporting a therapeutic role for EV-CPC^{ISX-9} in cardiac remodeling. Given that EV-CPC^{ISX-9} were found to be highly enriched in miR-373, we tested the effects of EV-CPC^{ISX-9} injection in the heart and miR-373 mimic treatment in vivo on miR-373 expression level and found that miR-373 expression level in the heart was increased and it significantly decreased fibrosis and improved cardiac function post MI. Moreover, the miR-373 mimic also promoted angiogenesis, which was likely mediated by its ability to activate HIF downstream signaling (39). These findings suggest that the anti-fibrotic effects of EV-CPC^{ISX-9} are, at least in part, mediated by miR-373, and they also support the notion that miR-373 mimic might represent a novel therapeutic strategy for controlling fibrosis and cardiac remodeling post infarction and perhaps in other disorders, such as diabetic cardiomyopathy.

The second major effect of EV-CPC^{ISX-9} was on cardiomyocyte proliferation in the infarcted myocardium. A previous study reported miR-294 (miR-290 cluster), the mouse homolog of human miR-371/372/373 cluster, had a strong effect on cardiac progenitor cell proliferation (40), and that its overexpression led to differentiation towards the mesendoderm lineage (17). It should be borne in mind, however, that these effects could also be attributed to other miRNAs present in the EV, including miR-302, miR-548, miR-512 and miR-367. Further studies are required to dissect the role of individual EV-CPC^{ISX-9} miRNAs in regulating cardiac fibrosis, cardiomyocyte proliferation, and other pathological events in the context of post-infarction remodeling.

### Conclusion

Several clinical and investigational reports have demonstrated the therapeutic application of cardiac progenitor cells for the treatment of ischemic heart. Consequently, these studies led to advance new cell free (EV) strategies to overcome the limitations of cell-based approaches with the same effectiveness and outcomes. The intracoronary administration of EV eliminates the need for open heart surgery for intramyocardial administration of stem cells. However, the promise of EV does not establish the fact whether their effect is continuous and permanent or future efforts should continue on strategies directed towards successful engraftment and survival of iPSC derived cardiac progenitors as a source for new myofiber growth and EV for paracrine effects as well.

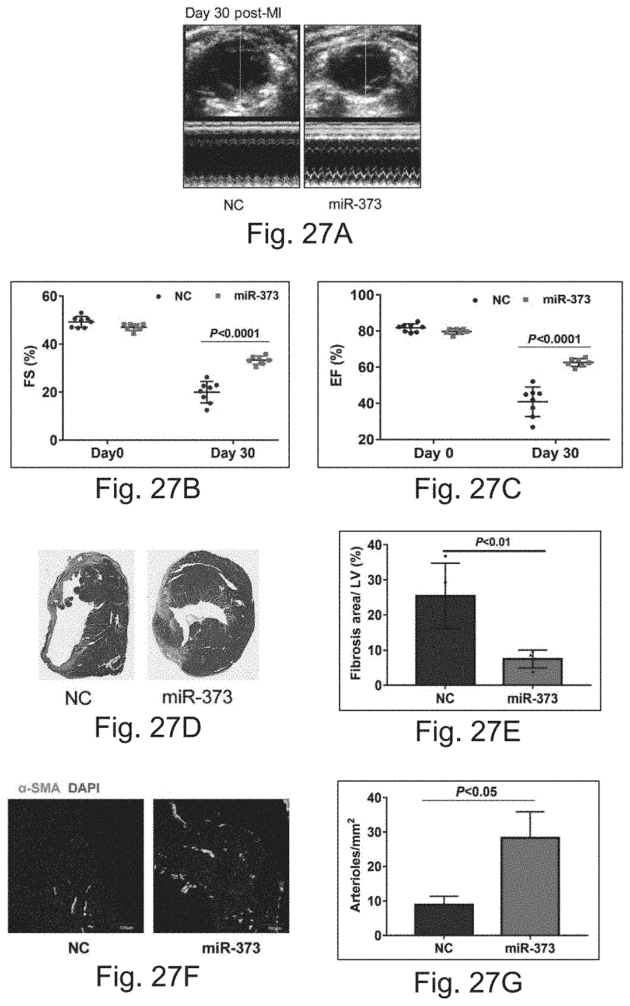

In summary (FIG. 28), we report that EV-CPC^{ISX-9} exhibit a unique miRNA profile, and that miR-373 is particularly highly enriched in these EV. EV-CPC^{ISX-9} elicit strong anti-fibrotic effects, which is attributed at least in part to their enrichment in miR-373. Treatment with EV-CPC^{ISX-9} in vivo reduced post-infarction fibrosis and remodeling, promoted cardiomyocyte proliferation and angiogenesis, and improved cardiac function, findings which were at least in part recapitulated by direct application of miR-373 mimic. These findings have important implications for understanding the paracrine mechanisms of stem cell function and advancing the field of cardiac stem cell therapeutics.

Data Availability:

The raw data of miRNA array is deposited in GEO database (GSE126347). Other data are available from the authors upon reasonable request.

Funding Statement:

This study was supported by National Institutes of Health grants, RO1 HL126516¹, RO1 HL134354² and RO1 AR070029² (to M Ashraf^{1,2}, Y Tang², N Weintraub²), and HL124097 and HL126949 (to N Weintraub²).

References for publication item inserted herein beginning in paragraph [0313]
1. Sanchis-Gomar F, Perez-Quilis C, Leischik R, Lucia A. Epidemiology of coronary heart disease and acute coronary syndrome. Annals of translational medicine 2016; 4:256.
2. Sahoo S, Losordo D W. Exosomes and cardiac repair after myocardial infarction. Circulation research 2014; 114:333-44.
3. Mirotsou M, Jayawardena T M, Schmeckpeper J, Gnecchi M, Dzau V J. Paracrine mechanisms of stem cell reparative and regenerative actions in the heart. Journal of molecular and cellular cardiology 2011; 50:280-9.
4. Der Sarkissian S, Levesque T, Noiseux N. Optimizing stem cells for cardiac repair: Current status and new frontiers in regenerative cardiology. World journal of stem cells 2017; 9:9-25.
5. Maxeiner H, Krehbiehl N, Muller A et al. New insights into paracrine mechanisms of human cardiac progenitor cells. European journal of heart failure 2010; 12:730-7.
6. Barile L, Moccetti T, Marban E, Vassalli G. Roles of exosomes in cardioprotection. European heart journal 2017; 38:1372-1379.
7. Ribeiro-Rodrigues T M, Laundos T L, Pereira-Carvalho R et al. Exosomes secreted by cardiomyocytes subjected to ischaemia promote cardiac angiogenesis. Cardiovasc Res 2017; 113:1338-1350.
8. Yang L, Soonpaa M H, Adler E D et al. Human cardiovascular progenitor cells develop from a KDR+embryonic-stem-cell-derived population. Nature 2008; 453:524-8.
9. Mauritz C, Martens A, Rojas S V et al. Induced pluripotent stem cell (iPSC)-derived Flk-1 progenitor cells engraft, differentiate, and improve heart function in a mouse model of acute myocardial infarction. European heart journal 2011; 32:2634-41.
10. Koninckx R, Daniels A, Windmolders S et al. The cardiac atrial appendage stem cell: a new and promising candidate for myocardial repair. Cardiovasc Res 2013; 97:413-23.
11. Agarwal U, George A, Bhutani S et al. Experimental, Systems, and Computational Approaches to Understanding the MicroRNA-Mediated Reparative Potential of Cardiac Progenitor Cell-Derived Exosomes From Pediatric Patients. Circulation research 2017; 120:701-712.
12. Lilian Grigorian-Shamagian W L, Soraya Fereydooni, Ryan C. Middleton, Jackelyn Valle, Jae Hyung Cho, Eduardo Marbán; Cardiac and systemic rejuvenation after cardiosphere-derived cell therapy in senescent rats. European heart journal 2017.
13. Barile L, Lionetti V, Cervio E et al. Extracellular vesicles from human cardiac progenitor cells inhibit cardiomyocyte apoptosis and improve cardiac function after myocardial infarction. Cardiovasc Res 2014; 103:530-41.
14. Sluijter J P, van Rooij E. Exosomal microRNA clusters are important for the therapeutic effect of cardiac progenitor cells. Circulation research 2015; 116:219-21.
15. Xuan W, Wang Y, Tang Y et al. Cardiac Progenitors Induced from Human Induced Pluripotent Stem Cells with Cardiogenic Small Molecule Effectively Regenerate Infarcted Hearts and Attenuate Fibrosis. Shock 2018.
16. Wu Y, Deng W, Klinke D J, 2nd. Exosomes: improved methods to characterize their morphology, RNA content, and surface protein biomarkers. Analyst 2015; 140:6631-42.
17. Rosa A, Papaioannou M D, Krzyspiak J E, Brivanlou A H. miR-373 is regulated by TGFbeta signaling and promotes mesendoderm differentiation in human Embryonic Stem Cells. Dev Biol 2014; 391:81-8.
18. Ong S G, Huber B C, Lee W H et al. Microfluidic Single-Cell Analysis of Transplanted Human Induced Pluripotent Stem Cell-Derived Cardiomyocytes After Acute Myocardial Infarction. Circulation 2015; 132:762-71.
19. Zhang Y, Cao N, Huang Y et al. Expandable Cardiovascular Progenitor Cells Reprogrammed from Fibroblasts. Cell stem cell 2016; 18:368-81.
20. Chong J J, Yang X, Don C W et al. Human embryonic-stem-cell-derived cardiomyocytes regenerate non-human primate hearts. Nature 2014; 510:273-7.
21. Lalit P A, Salick M R, Nelson D O et al. Lineage Reprogramming of Fibroblasts into Proliferative Induced Cardiac Progenitor Cells by Defined Factors. Cell stem cell 2016; 18:354-67.
22. Liu M L, Nagai T, Tokunaga M et al. Anti-inflammatory peptides from cardiac progenitors ameliorate dysfunction after myocardial infarction. J Am Heart Assoc 2014; 3:e001101.
23. Menasche P, Vanneaux V, Fabreguettes J R et al. Towards a clinical use of human embryonic stem cell-derived cardiac progenitors: a translational experience. European heart journal 2015; 36:743-50.
24. Herrmann J L, Wang Y, Abarbanell A M, Weil B R, Tan J, Meldrum D R. Preconditioning mesenchymal stem cells with transforming growth factor-alpha improves mesenchymal stem cell-mediated cardioprotection. Shock 2010; 33:24-30.
25. Crisostomo P R, Wang M, Markel T A et al. Stem cell mechanisms and paracrine effects: potential in cardiac surgery. Shock 2007; 28:375-83.
26. Cha D J, Franklin J L, Dou Y et al. KRAS-dependent sorting of miRNA to exosomes. Elife 2015; 4:e07197.
27. Wei F, Cao C, Xu X, Wang J. Diverse functions of miR-373 in cancer. J Transl Med 2015; 13:162.
28. Shen E, Diao X, Wang X, Chen R, Hu B. MicroRNAs involved in the mitogen-activated protein kinase cascades pathway during glucose-induced cardiomyocyte hypertrophy. Am J Pathol 2011; 179:639-50.
29. Fang L, Ellims A H, Moore X L et al. Circulating microRNAs as biomarkers for diffuse myocardial fibrosis in patients with hypertrophic cardiomyopathy. J Transl Med 2015; 13:314.
30. Knipe R S, Probst C K, Lagares D et al. The Rho Kinase Isoforms ROCK1 and ROCK2 Each Contribute to the Development of Experimental Pulmonary Fibrosis. Am J Respir Cell Mol Biol 2018; 58:471-481.
31. Shimizu Y, Dobashi K, Sano T, Yamada M. ROCK activation in lung of idiopathic pulmonary fibrosis with oxidative stress. Int J Immunopathol Pharmacol 2014; 27:37-44.
32. Shimizu T, Narang N, Chen P et al. Fibroblast deletion of ROCK2 attenuates cardiac hypertrophy, fibrosis, and diastolic dysfunction. JCI Insight 2017; 2.
33. Loffredo F S, Steinhauser M L, Jay S M et al. Growth differentiation factor 11 is a circulating factor that reverses age-related cardiac hypertrophy. Cell 2013; 153:828-39.
34. Sinha M, Jang Y C, Oh J et al. Restoring systemic GDF11 levels reverses age-related dysfunction in mouse skeletal muscle. Science 2014; 344:649-52.
35. Zimmers T A, Jiang Y, Wang M et al. Exogenous GDF11 induces cardiac and skeletal muscle dysfunction and wasting. Basic Res Cardiol 2017; 112:48.
36. Egerman M A, Cadena S M, Gilbert J A et al. GDF11 Increases with Age and Inhibits Skeletal Muscle Regeneration. Cell Metab 2015; 22:164-74.
37. Rinaldi F, Zhang Y, Mondragon-Gonzalez R, Harvey J, Perlingeiro R C R. Treatment with rGDF11 does not improve the dystrophic muscle pathology of mdx mice. Skelet Muscle 2016; 6:21.
38. Pons M, Koniaris L G, Moe S M, Gutierrez J C, Esquela-Kerscher A, Zimmers T A. GDF11 induces kidney fibrosis, renal cell epithelial-to-mesenchymal transition, and kidney dysfunction and failure. Surgery 2018; 164:262-273.
39. Crosby M E, Devlin C M, Glazer P M, Calin G A, Ivan M. Emerging roles of microRNAs in the molecular responses to hypoxia. Curr Pharm Des 2009; 15:3861-6.
40. Khan M, Nickoloff E, Abramova T et al. Embryonic stem cell-derived exosomes promote endogenous repair mechanisms and enhance cardiac function following myocardial infarction. Circulation research 2015; 117:52-64.

Figure Legends for Material Discussed Beginning in Paragraph 0298

FIG. 22. Characterization of EV from iPSC and CPC^{ISX-9}. (A) Secretion of EV from the CPC^{ISX-9} as imaged by electron microscopy. Inset shows higher magnification of secreted EV (small black arrows). Blue arrows point to EV exiting from the cells. Bar=1 μm. (B) EV isolated from iPSC and CPC^{ISX-9} visualized by transmission electron microscopy (TEM). Scale bar=200 nm. (C) Representative images of western blot for Tsg101, CD9, Hsp70, Flotillin-1, and Calnexin in EV lysates. C: cell lysate; E: EV. (D) Average size of EV as measured by TRPS. No significant difference in average size of EV from iPSC and CPC^{ISX-9} was observed. (E) Representative graph of size distribution of EV from iPSC and CPC^{ISX-9} as detected by TRPS.

FIG. 23 miRNA expression profiling and validation of microarray data. (A) Outline of experimental procedure. (B) Heatmap analysis of microarray data showing significant upregulation of miRNAs in EV-CPC^{ISX-9} compared with those in EV-iPSC, EV-EB or EV-control-CPC. Red or blue colors indicate differentially up- or downregulated miRNA, respectively (P<0.05). n=3. (C) Biological process of Gene Ontology (GO) enrichment analysis based on miRNA-targeted genes. GO enrichment was analyzed with mirPath v.3 software. GO biological process includes biological processes, molecular function and cellular component of upregulated and downregulated genes. (D) Validation of microarray data using real-time PCR. Quantitative results showing significant expression of miR-373, miR-367, miR-520, miR-548ah, and miR-548q in EV-CPC^{ISX-9} RNA samples were from three individual experiments. *P<0.001.

FIG. 24 Fibrotic gene expression in fibroblasts after TGF-β stimulation. (A) Effects of EV-CPC^{ISX-9} on fibrotic gene expression: role of miR-373. n=6 (B) Transdifferentiation of lung fibroblasts and dermal fibroblasts into myofibroblast by hypoxia for 72 h as detected by immunostaining for α-smooth actin (α-SMA): effects of EV-CPC^{ISX-9} and miR-373 mimic pretreatment. Bar=50 μm. (C) Schematic representation of the luciferase reporter constructs. (B) Sequence alignment of miR-373 with the human wild type (WT) ROCK-2 3′-UTR and GDF-113′-UTR and mutated reporters. The seed sequence (Red) is highlighted. (D) Relative luciferase activity (relative, firefly luciferase activity/Renilla luciferase activity) of 293FT cells co-transfected with WT 3′-UTR-ROCK-2 or GDF-11 and mutant 3′-UTR-ROCK-2 or GDF-11 and miR-373 mimics vs. NC. ** P<0.01, n=4. UTR, untranslated region; miRNA, microRNA; NC, negative control; WT, wild type. (E) 72 h hypoxia increased GDF-11 and ROCK-2 mRNA expression in lung fibroblasts: effects of pretreatment with miR-373 mimic. *** P<0.001. n=6.

FIG. 25 CPC^{ISX-9}-derived EV promoted cardiomyocyte proliferation and angiogenesis after myocardial infarction (MI) in mice. (A) Representative image of ki67 positive cardiomyocytes (cTnT positive) in EV-CPC^{ISX-9} treated mouse hearts 30 days after MI. Bar=50 μm. (B) Quantitative estimate of proliferating cardiomyocytes as determined by Ki67 staining in peri-infarct region 30 days after myocardial infarction. PBS group: n=940 cardiomyocytes from 3 hearts; EV-iPSC group: n=950 cardiomyocytes from 3 hearts; EV-CPC^{ISX-9} group, n=951 cardiomyocytes from 3 hearts. * vs. PBS group, P<0.05; # vs. EV-iPSC group, P<0.05. (C) Representative images of arteriole density in peri-infarct area 4 weeks after MI. Arterioles were identified by α-SMA positive staining (green) of vascular structures. Bar=100 μm. (D) Quantitative analysis of arteriole density in different treatment groups. * vs. PBS group, P<0.05; # vs. EV-iPSC group, P<0.05, n=3.

FIG. 26 CPC^{ISX-9} derived EV reversed cardiac remodeling in infarcted mice. (A) Representative M mode echocardiography images from three groups 30 days after MI. LVDs (B), LVDd (C), EF (D) and FS (E) are shown. * vs. PBS group, P<0.05; # vs. EV-iPSC group, P<0.05, PBS group: n=10, EV-iPSC group, n=9, EV-CPC^{ISX-9}, n=11. EF, ejection fraction; FS, fractional shortening; LVDd, diastolic left ventricular dimensions; LVDs systolic left ventricular dimensions. (F) Representative Masson's trichrome-stained sections of hearts from three groups. (G) Quantitative estimate of fibrosis. * vs. PBS group, P<0.05; # vs. EV-iPSC group, P<0.05, n=4.

FIG. 27 miR-373 mimic improved cardiac function and angiogenesis and attenuated cardiac fibrosis after MI. (A) Representative M mode echocardiography images from miRNA mimic negative control (NC) treated mice and miR-373 mimic treated mice 30 days post-MI. FS (B) and EF (C) are shown; (P<0.001), n=8 in NC group and n=7 in miR-373 mimic group. (D) Representative Masson's trichrome-stained sections of hearts from NC treated mice and miR-373 mimic mice. (E) Quantitative analysis of fibrosis post MI. (F) Vessel density was assessed by α-SMA positive staining (green) of vascular structures. Bar=100 μm. (G) Quantitative estimate of arteriole density. P<0.05. n=3 in each group. (H) Schematic depiction of mechanisms of protection by EV-CPC^{ISX-9}: role of miR-373 in suppressing fibrosis by targeting two genes, GDF-11 and ROCK-2 and inhibiting myofibroblast differentiation. Myocyte proliferation and angiogenesis were also promoted by EV-CPC^{ISX-9}.

FIG. 28 Schematic depiction of mechanisms of protection by EV-CPC^{ISX-9}: role of miR-373 in suppressing fibrosis by targeting two genes, GDF-11 and ROCK-2 and inhibiting myofibroblast differentiation. Myocyte proliferation and angiogenesis were also promoted by EV-CPC^{ISX-9}.

The following publication is incorporated herein in its entirety

### Human iPS Cells Derived Skeletal Muscle Progenitor Cells Promote Myoangiogenesis and Restore Dystrophin in Duchenne Muscular Dystrophic Mice

Abstract

Background and Objective:

Duchenne muscular dystrophy (DMD) is caused by mutations of the gene that encodes the protein dystrophin. Loss of dystrophin leads to severe and progressive muscle-wasting in both skeletal and heart muscles. Human induced pluripotent stem cells (hiPSCs) and their derivatives offer important opportunities to treat a number of diseases. Here, we investigated whether givinostat, a histone deacetylase inhibitor (HDACi), could reprogram hiPSCs into muscle progenitor cells (MPC) for DMD treatment.

Methods and Results:

MPC generated by CHIR99021 and givinostat (Givi) small molecules from multiple hiPSCs expressed myogenic makers (Pax7, desmin) and were differentiated into myotubes expressing MF20 upon culture in specific differentiation medium. These MPC exhibited superior proliferation and migration capacity determined by CCK-8, colony and migration assays compared to control-MPC generated by CHIR99021 and fibroblast growth factor (FGF). Upon transplantation in hind limb of Mdx/SCID mice with cardiotoxin (CTX) induced injury, these MPC showed higher engraftment and restoration of dystrophin than treatment with control-MPC and human myoblasts. In addition, treated muscle with these MPC showed significantly limited infiltration of inflammatory cells and reduced muscle necrosis and fibrosis. A number of these cells were engrafted under basal lamina expressing Pax7, which were capable of generating new muscle fibers after additional injury. Extracellular vesicles released from these cells promoted angiogenesis after reinjury.

### Conclusion

We successfully generated highly expandable and integration free MPC from multiple hiPS cell lines using CHIR99021 and Givi. Givinostat induced MPC showed marked and impressive regenerative capabilities and restored dystrophin in injured tibialis muscle compared to control MPC. Additionally, MPC generated by Givi also seeded the stem cell pool in the treated muscle. It is concluded that hiPSCs pharmacologically reprogrammed into MPC with a small molecule, Givi with anti-oxidative, anti-inflammatory and muscle gene promoting properties might be an effective cellular source for treatment of muscle injury and restoration of dystrophin in DMD.

Keywords: Duchenne Muscular Dystrophy; Human induced pluripotent stem cells; muscle progenitor cells; histone deacetylase inhibitor, angiogenesis

Introduction

Duchenne muscular dystrophy (DMD) is caused by mutations of the gene that encodes the protein dystrophin. Loss of dystrophin leads to severe and progressive muscle-wasting in both skeletal and heart muscles. Cell replacement gives a promising hope for DMD therapy. Satellite cells (SCs) are endogenous skeletal muscle stem cells, which are responsible for muscle maintenance and muscle regeneration after injury (1,2). A previous study reported that xenotransplantation of human SCs into mice achieved efficient engraftment and populated the satellite niche (3). However, a biopsy is needed for procurement of SCs. In addition, freshly isolated SCs progeny though can be propagated in vitro but their transplantation potential becomes limited during in vitro expansion (4-6). Therefore, procurement of larger number of SCs for transplantation becomes an obstacle for clinical application. Human induced pluripotent stem cells (hiPSCs) derived derivatives offer important sources to treat a number of diseases. Efforts have been made in the past few years for generation of muscle progenitor cells (MPC) from hiPSCs either by genetic modification or small molecules. Nevertheless, generation of MPC from hiPSCs by viral vectors remains a safety concern. High percentage of Pax7 positive MPC can be generated from hiPSC by small molecules (CHIR99021, LDN19389 and FGF) (7,8), but their limited engraftment was observed in vivo upon transplantation (9). Interestingly, it has been recently reported that MPC can be generated from teratoma which showed high engraftment efficiency in muscle dystrophy model (10). However, human teratoma derived MPC poses safety concerns for clinical application. Therefore, it seems more appropriate to look for alternate approaches for inducing MPC from hiPSCs with high engraftment and differentiation properties.

Givinostat is a histone deacetylase inhibitor (HDACi) that has been shown to increase muscle regeneration in a mouse model of DMD (11). Interestingly, most of the beneficial effects of HDACi arise from its ability to redirect fibroadipogenic lineage commitment toward a myogenic fate (12). Using genome-wide Chip-seq analysis in myoblasts, it was demonstrated that HDACi induced myogenic differentiation program in myoblasts (i.e., Myosin 7, Enolase 3 and Myomesin1) (13). Therefore, here we propose that Givi could reprogram hiPSCs into MPC for DMD treatment.

Methods

Human iPSC Culture

The Human iPSC cell lines from ATCC Company CYS0105 and DYS0100 were used. CYS0105 was reprogrammed from human cardiac fibroblasts of a 72 years old healthy donor, while DYS0100 was reprogrammed from human foreskin fibroblasts of a normal newborn. DMD-iPS cell line (SC604A MD) was purchased form SBI Company, which was generated from a DMD patient with Exon 3-7 deletion of dystrophin. The forth iPS cell line was reprogrammed from human dermal fibroblasts (CC-2511, Lonza) of a 45 years old healthy donor in our lab using Cyto Tune™ iPS 2.0 sendai reprogramming kit (A16517, Thermo fisher Scientific) as previously described (14). iPSCs were grown and maintained on vitronectin coated six-well plate in mTeSR1 medium (Stem Cell Technologies) with daily change.

Differentiation Protocols to Generate Muscle Progenitor Cells (MPC) and their Characterization

Human iPSCs at passage 20-30 were used for conversion to MPC. Human iPSCs were dissociated into single cells using Accutase (Stem Cell Technologies) at 37 for 10 min and then were seeded onto a vitronectin-coated six-well plate at 3×10⁵ cell/well in mTeSR1 supplemented with 5 μM ROCK inhibitor (Y-27632, Stem Cell Technology) for 24 h. Afterwards cells were switched into E6 medium (Thermo Fisher Scientific) supplemented with CHIR99021 (10 μM) for two days followed by Givi (100 nM) for 5 days. The differentiating cells were cultured in E6 medium for 7 days. The schematic outline is shown in FIG. 1A. At Day 14, cells were replated on 0.1% galectin coated coverslips and expression of Pax7 and desmin were analyzed by immunostaining. MPC were expanded in SKGM-2 medium plus FGF-2 (2.5 ng/ml) and cells at passage 2-4 were used for experiments. Here, we referred the givinostat induced MPC as Givi-MPC. To further enhance muscle differentiation, at Day 14, cultured cells were replated and switched into high glucose DMEM medium supplemented with 2% horse serum (Thermo fisher Scientific) and 1% ITS (Thermo fisher Scientific) for 7 days. Immunostaining of cultured cells for MF20 was performed. To generate control-MPC (7), a previously reported method using only CHIR99021 was used. The schematic outline is shown in supplemental FIG. S1. Control-MPC were expanded using the same condition as Givi-MPC and passage 2-4 of cells were used for experiments.

CCK-8 Assay for Proliferation

CCK-8 assay was used for evaluation of cell proliferation. Briefly, 2000 cells were seeded into 96 well plate per well and cell proliferation was analyzed at 0 h, 24 h, 48 h and 72 h respectively by using CCK-8 kit (ab228554, abcam) according to the manufacturer protocol.

Colony Formation

Thirty cells (single cell) were seeded in one well of six-well plate. After 7 days, cells were stained with crystal violet dye. Number of colonies and size of cell growth were analyzed and compared between control-MPC and Givi-MPC groups.

Cell Migration

For cell migration experiment, human myoblasts, control-MPC and Givi-MPC were seeded in 35 mm dish with culture-insert 2 well (ibidi company) at 1×10⁵/ml concentration in SKGM-2 medium with 2% Fetal Bovine Serum (FBS). The next day, a confluent layer was observed and culture-inserts were removed, and after 24 h the number of migrated cells were analyzed.

Human Endothelial Cell and Human Myoblast Culture

Human aortic endothelial cells (HAEC, CC-2535) and human skeletal myoblasts (HSMM-Muscle Myoblasts, CC-2580) were obtained from Lonza Company. HAEC were maintained in endothelial cell growth medium V-2 (213-500, CELL APPLICATIONS, Inc.) and cells at passage 2-6 were used for experiments. Human myoblasts were maintained in SKGM-2 medium (Lonza) and cells at passage 2-4 were used for experiments.

Cardiotoxin Injury and Cell Transplantation

Animal experiments were carried out according to experimental protocol approved by the Augusta University Animal Care and Use Committee. 6-8 weeks old Mdx/SCID mice (Stock No: 018018, The Jackson Laboratory) were used in the present study. One-day prior to cell transplantation, mice were anaesthetized using 2% isoflurane and tibialis anterior (TA) muscle was injured with 50 μl of 10 μM cardiotoxin (Naja mossambica-mossambica, Sigma). For cell transplantation experiments, control-MPC and Givi-MPC were differentiated from the same human iPS cell line, DYS0100. For transplantation, myoblast, control-MPC and Givi-MPC were dissociated using Accutase (Stem Cell Technologies) and resuspended in Dulbecco's phosphate-buffered saline (DPBS) at 1×10⁵ per 20 μl. Cells were injected into the left TA muscle while the same volume of DPBS was injected into the right TA as control. In some cases, cells were transfected with Green Fluorescent Protein (GFP) Lentivirus (abm company, Canada) for cell tracking. Some Mdx/SCID mice transplanted with Givi-MPC were subjected to CTX reinjury at 2M after first injury and cell transplantation.

Immunostaining for Cells

Cells were fixed with 4% PFA, and blocked with 10% FBS, followed by incubation with anti-Pax7 antibody (ab187339, abcam, 1:300), anti-desmin antibody (ab32362, abcam, 1:500) and anti-Myosin Heavy Chain Antibody (MF20) antibody (Novus, MAB4470, 1:200) respectively at 4° C. overnight and secondary antibody conjugated to Alexa Fluor 594 or Alexa Fluor 488 (Life Technologies) at room temperature for 1 h. Images were taken by a florescent microscope (Olympus, Japan).

Immunostaining for Muscle Sections

After 7 days or 30 days of cell transplantation, Mdx/SCID mice were euthanized and TA muscles were harvested and fixed with 4% paraformaldehyde (PFA) for 1 h at room temperature and then immersed in 30% sucrose overnight at 4° C. At day two, hearts were cryopreserved in an optical cutting temperature (OCT) compound (Tissue Tek) at −80° C. TA muscle samples were sliced into 5-μm-thick frozen cross-sections using a Leica CM3050 cryostat. Muscle sections were incubated with primary antibodies including Laminin (L9393, Sigma, 1:500), dystrophin (D8168, Sigma, 1:200), human specific laminin (LAM-89, Novus, 1:200), GFP (#2956, Cell Signal Technologies, 1:500), dystrophin (ab15277, abcam, 1:200), human nuclear antigen (NBP2-34342, Novus, 1:100), CD68 (NB600-985, Novus, 1:200) and CD31 (NB600-562, Novus, 1:200) at 4° C. overnight respectively and anti-rabbit/mouse secondary antibodies conjugated to Alexa Fluor 594 or Alexa Fluor 647 or Alexa Fluor 488 (Life Technologies) at room temperature for 1 h. Images were taken using a confocal microscope (FV1000, Olympus, Japan). For cell engraftment quantification, 4 sections at 150 μm interval in each TA muscle were analyzed. Dystrophin or laminin staining was used to define the physical boundaries of muscle fibers. The number of muscle fibers and cross-section area were measured using Image J with the colocalization plugin (NIH). Capillary density was assessed in 4 sections cut at 150 μm interval by counting CD31 positive vascular structures using a fluorescence microscope at a magnification of 400×. The number of capillaries in each TA muscle was expressed as the number of capillaries per field (0.2 mm²). For quantification of inflammatory cells, number of CD68 positive cells were counted in 3 sections cut at 150 μm interval after 7 days' post cell transplantation and was expressed as the number of CD68 positive cells per field (0.2 mm²). Staining of presynaptic marker α-bungarotoxin (α-BTX) was carried out using α-bungarotoxin, Alexa Fluor™ 594 conjugate (Invitrogen) according to the manufacturer's instruction.

Histology

Histological staining was performed at Electron Microscopy and Histology Core of Augusta University. After 7 days or 30 days of cell transplantation, TA muscle were harvested and embedded in paraffin. 5-μm-thick sections of TA muscle were cut and stained with hematoxylin and eosin (H and E), Masson trichrome and Sirius red according to the manufacturer protocol (abcam). Images were taken by a vertical microscope (Olympus, Japan). Fibrosis and necrosis were determined using the ImageJ software (NIH) and expressed as the ratio of total area of the cross-section and normalized with the ratio of control lateral TA muscle section. Myofiber necrosis was identified with fragmented sarcoplasm (15) and/or increased inflammatory cell infiltration, and was measured using non-overlapping tile images of transverse muscle sections that provided a view of the entire muscle cross section.

Isolation of Extracellular Vesicles (EV)

EV were isolated using size exclusion column method as we described previously (16). Briefly, conditioned media was collected and EV were isolated by centrifugation at 3000 rpm for 30 min to remove cells and debris, followed by filtration through a 0.22 μm filter to remove the remaining debris. Then the medium was further concentrated using Amicon Ultra-15 100 KDa centrifugal filter units (Millipore). Isolation of EV in the concentrated medium was carried out through qEV size exclusion columns (Izon Science). EV fractions were collected and concentrated by Amicon Ultra-4 10 KDa centrifugal filter (Millipore). The purified EV were stored at −80° C. and subsequently characterized by particle size and electron microscopy.

Concentration and Particle Size Measurement with Tunable Resistive Pulse Sensing

Particle size and concentration were analyzed using tunable resistive pulse sensing (TRPS) technique with a qNano instrument (Izon Science) as described in previous studies (16,17). Briefly, the number of particles were counted (at least 600 to 1000 events) at 20 mbar pressure. Beads CPC200 (200 nm) were used for calibration. Data were analyzed using Izon Control Suite software.

Transmission Electron Microscopy (TEM)

Tissue samples were processed for TEM by the Electron Microscopy and Histology Core Laboratory at Augusta University as described previously (16). Briefly, EV suspension was fixed with an equal volume of 8% paraformaldehyde to preserve ultrastructure. Ten μl of suspended/fixed exosomes was applied to a carbon-formvar coated 200 mesh copper grid and allowed to stand for 30-60 seconds. The excess was absorbed by Whatman filter paper. 10 μl of 2% aqueous uranyl acetate was added and treated for 30 seconds. Grids were allowed to air dry before being examined in a JEM 1230 transmission electron microscope (JEOL USA Inc., Peabody, Mass.) at 110 kV and imaged with an UltraScan 4000 CCD camera & First Light Digital Camera Controller (Gatan Inc., Pleasanton, Calif.).

RNA Extraction and PCR Array

Total RNA from cells was isolated using miRNeasy Kit (Qiagen). Reverse transcription was performed using QuantiTect Reverse Transcription kit (Qiagen). Human cell motility RT2 profiler PCR Array (Qiagen) for control-MPC and Givi-MPC was performed. Data was analysed using RT2 Profiler PCR Array Data Analysis Webportal (Qiagen). Genes with a fold change >2.0 were considered overexpressed.

RNA Extraction from EV and miRNA Array Analysis

Total RNA from EV was isolated using miRNeasy Micro Kit (Qiagen). The miRNA Array analysis was performed in the Integrated Genomics and High Performance Computer Server center at Augusta University. RNA purity and concentration were evaluated by spectrophotometry using Nanodrop ND-1000 (Thermo Fisher Scientific). Quality and the related size of small RNA was assessed by the Agilent 2100 Bioanalyzer (Agilent Technologies, Santa Clara, Calif.). 130 ng of total RNA was labeled with biotin using the FlashTag Biotin HSR RNA Labeling Kit (Applied Biosystems) according to manufacturer's procedure. The labeled samples were then hybridized to the GeneChip miRNA 4.0 array (Thermofisher) that contains 2,578 and 2,025 human mature and premature miRNA, respectively. Array hybridization, washing, and scanning of the arrays were carried out according to Affymetrix's recommendations. Data was obtained in the form of CEL file. The CEL files were imported into Partek Genomic Suites version 6.6 (Partek, St. Louis, Mo.) using standard import tool with RMA normalization. The differential expressions were calculated using ANOVA of Partek Package.

Tube Formation Assay

Human aortic endothelia cells (HAEC, 1×10⁵ cells/well) were seeded on Matrigel (Corning) in a 24-well plate and treated with or without 1 μg EV from different groups of Givi-MPC, control-MPC and human myoblast in EGM-2V basal medium (Lonza). After 16 h, cells in Matrigel were stained with Calcein AM, and images were taken with fluorescent microscope. Tube formation was analysed by Image J software with the angiogenesis analyzer plugin (NIH).

Statistical Analysis

Data were expressed as mean SD. After test for normality, statistical analysis of differences among different groups was compared by ANOVA with Bonferroni's correction for multiple comparisons. Percentage of different size of colony was compared using Chi-squared test. The Differences were considered statistically significant at P<0.05. Statistical analyses were performed using Graphpad Prism 6.0 (Chicago, US).

Results

Generation of muscle progenitor cells from human iPSC using small molecules

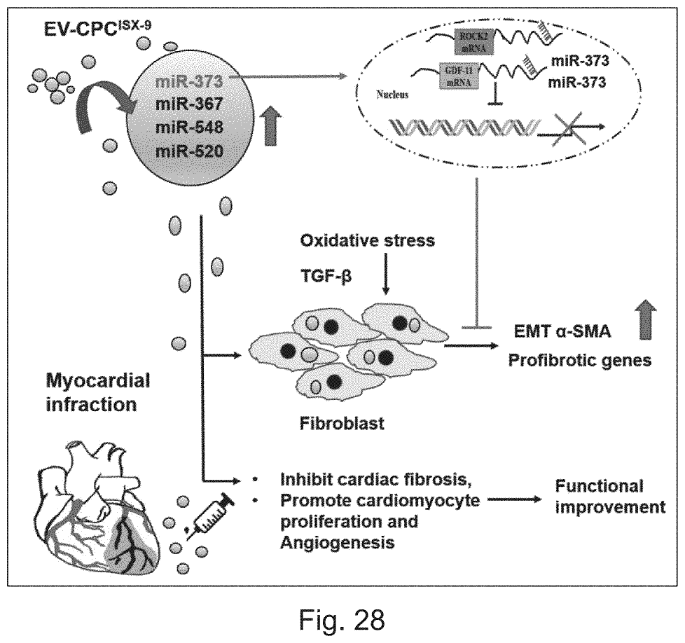

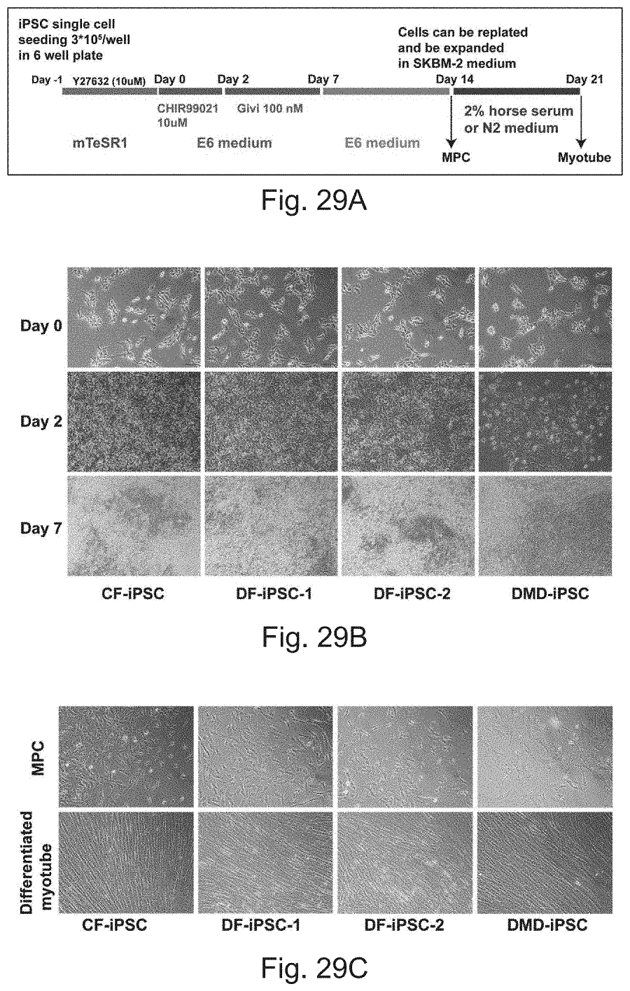

As outlined in FIG. 1A, we used 3 iPSC lines from healthy donors with different ages, and one iPSC line from DMD patient with frameshift deletions of exons 3-7 in the dystrophin gene for MPC generation. After 2 days treatment with CHIR99021, the morphology of the differentiating cells from 4 cell lines was dramatically altered indicative of epithelial to mesenchymal transition (EMT) (FIG. 29B). Following treatment with givinostat for 5 days, cells became confluent and clustered (FIG. 29B). FIG. 1C showed the morphology of differentiated MPC after replating and terminal muscle differentiation for 7 days in 2% horse serum differentiation medium. Using immunostaining, the MPC derived from 4 iPSC lines expressed the myogenic markers Pax7 and desmin. The MPC during terminal differentiation exhibited elongated shape (FIG. 29C) and expressed MF20 (FIG. 30B), indicating their myogenic differentiation potential.

Givinostat Induced MPC (Givi-MPC) Expressed High Proliferation and Motility Properties In Vitro

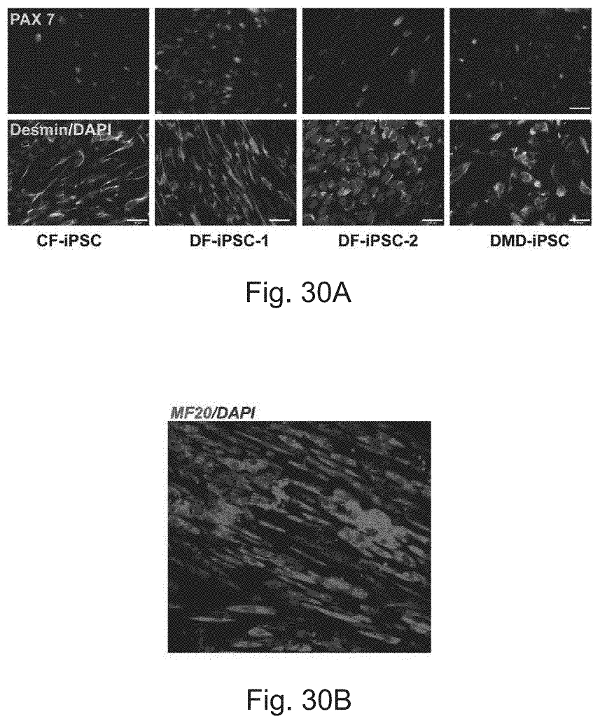

Next, we explored whether MPCs were proliferative and possessed self-renewal and motility properties. Using migration assay, compared to normal adult human myoblasts or control-MPC, Givi-MPC exhibited superior migration capability in low serum medium culture (FIG. 31A) with highest number migrated compared to other MPCs (FIG. 31B). Genes related to migration as determined by cell motility PCR array increased multifold in Givi-MPC as compared with MPCs (ITGA4 (25.61 fold), RAC2 (7.48 fold), FGF2 (6.75 fold) AND ENAH (5.53 fold)). FIG. 31C and FIG. 31D show heatmap and the list of upregulated genes related to migration (>2 fold). In addition, CCK-8 assay at 48 h and 72 h time points Givi-MPC showed higher OD value compared with human myoblasts and control-MPC (FIG. 31E). These data suggest that Givi-MPC possess better self-renewal potential. Colonies formed by Givi-MPC were bigger and had a higher cell density compared to control-MPC (FIG. 31F). Quantitative data (FIGS. 31G and 31H) showed that Givi-MPC formed more colonies with higher density of cells (>200 cells) compared to control-MPC. These data support the notion that Givi-MPC possessed highly proliferation and self-renewal capabilities.

In Vivo Engraftment of Givi-MPC Restores Dystrophin and Integrated into the Recipient Environment

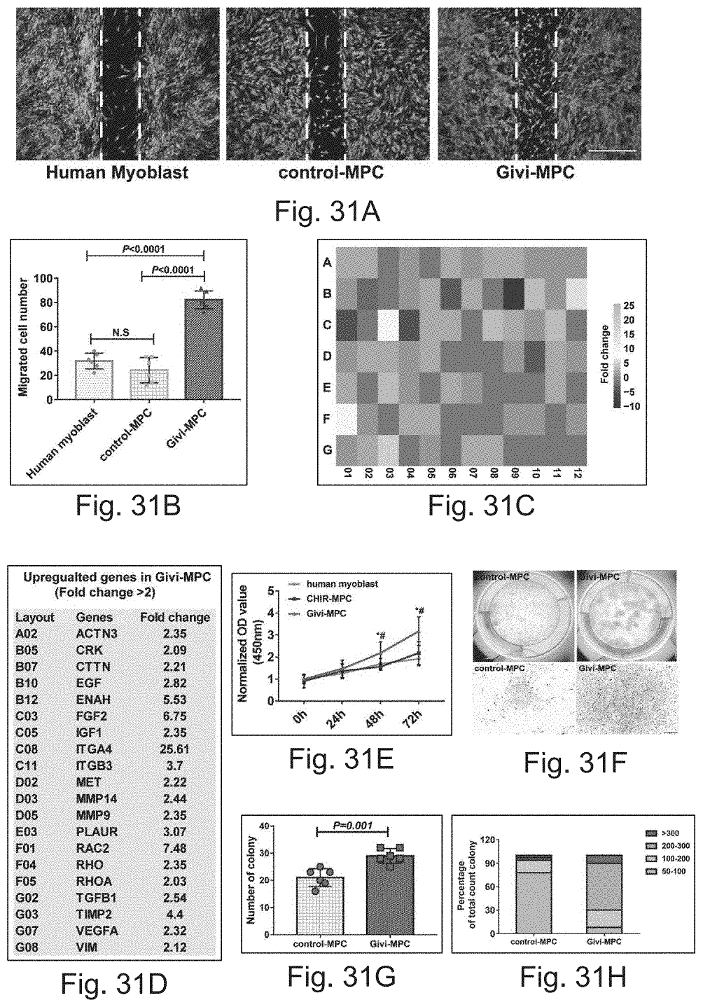

We transplanted human myoblast, control-MPC and Givi-MPC into Mdx/SCID mice with CTX injury, respectively. One-month post-transplant, Givi-MPC showed increased engraftment capacity and restoration of dystrophin than treatment with control-MPC and human myoblasts (FIGS. 32A and 32B). FIG. S2 showed the engrafted Givi-MPC (GFP positive) expressed dystrophin. Quantitative data showed Givi-MPC treated TA muscle had significantly higher number of dystrophin positive muscle fibers (FIG. 32C) and GFP and human laminin double positive muscle fibers (FIG. 32D). To determine the functionality of the newly formed muscle fibers from Givi-MPC, we tested whether they were integrated into the recipient environment with innervation. Positive staining of presynaptic marker α-BTX was observed in close proximity to dystrophin positive muscle fibers in Givi-MPC treated TA muscle, suggesting the presence of neuromuscular junction in these muscle fibers (FIG. 32E).

Givi-MPC Limited Inflammation, Muscle Necrosis and Reduced Fibrosis in Mdx/SCID Mice Post CTX Injury

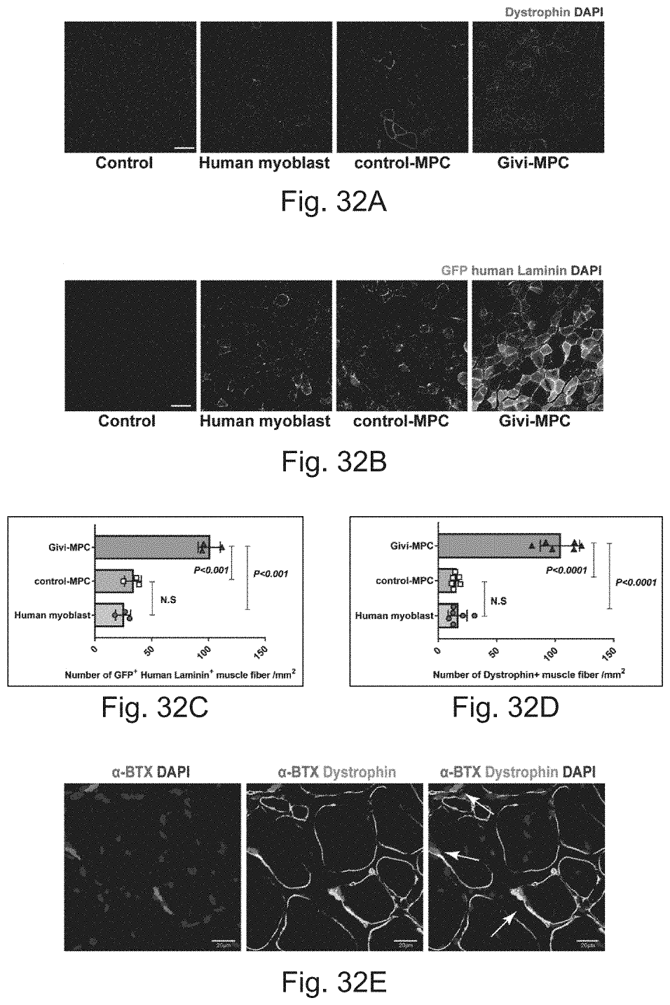

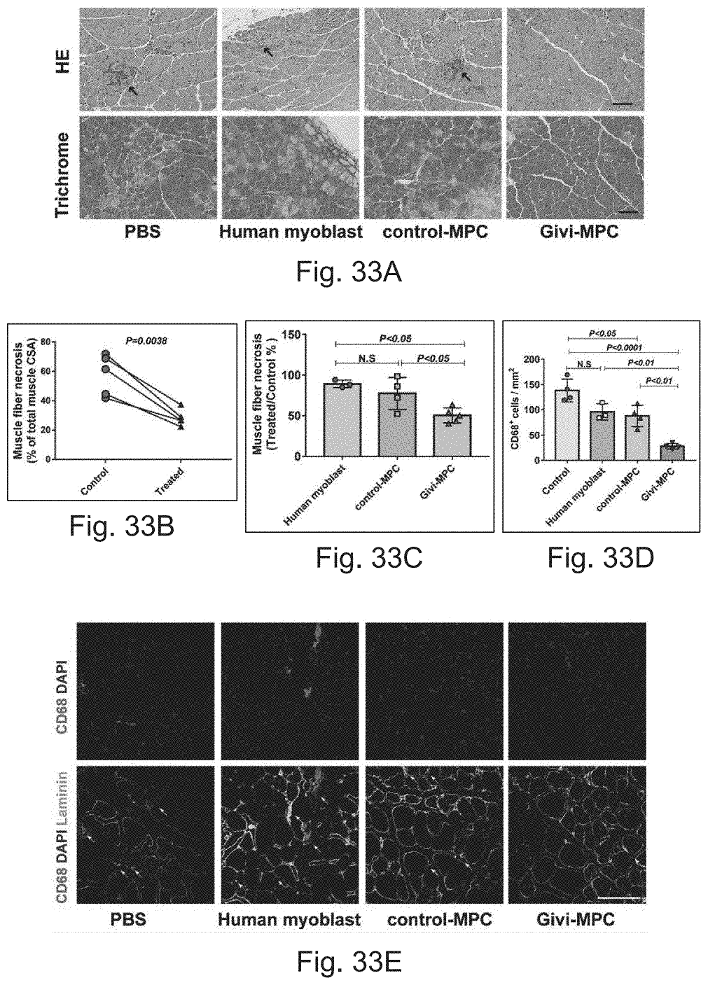

Hematoxylin and eosin and trichrome Masson staining revealed infiltration of inflammatory cells, and necrotic muscle fibers in Mdx/SCID mice 7 days' post CTX injury (FIG. 33A). A significant decrease in muscle necrosis was observed in Givi-MPC treated TA muscle compared to collateral PBS treated TA muscle (FIG. 33B). Amongst different MPCs which were transplanted, Givi-MPC reduced muscle necrosis the most (FIG. 33C) with reduced number of CD68 positive macrophages as compared with human myoblast and control-MPC treated tissue (FIG. 33D, E) 7 days' post CTX injury. In the muscle following 1M post CTX injury, transplantation of human myoblasts, control-MPC and Givi-MPC significantly decreased muscle necrosis compared to PBS treated collateral TA muscle (FIG. 34A-34D). No significant difference in muscle fiber necrosis was observed between human myoblasts and control-MPC treated TA muscle. However, Givi-MPC treatment resulted in reduced necrosis area compared to other MPCs treatment (FIG. 34E). Similarly, Givi-MPC transplantation reduced collagen deposits (red) compared to PBS, human myoblasts and control-MPC treated muscle (FIG. 34F, 34G-34I).

Givi-MPC Repopulated the Muscle Stem Cell Pool

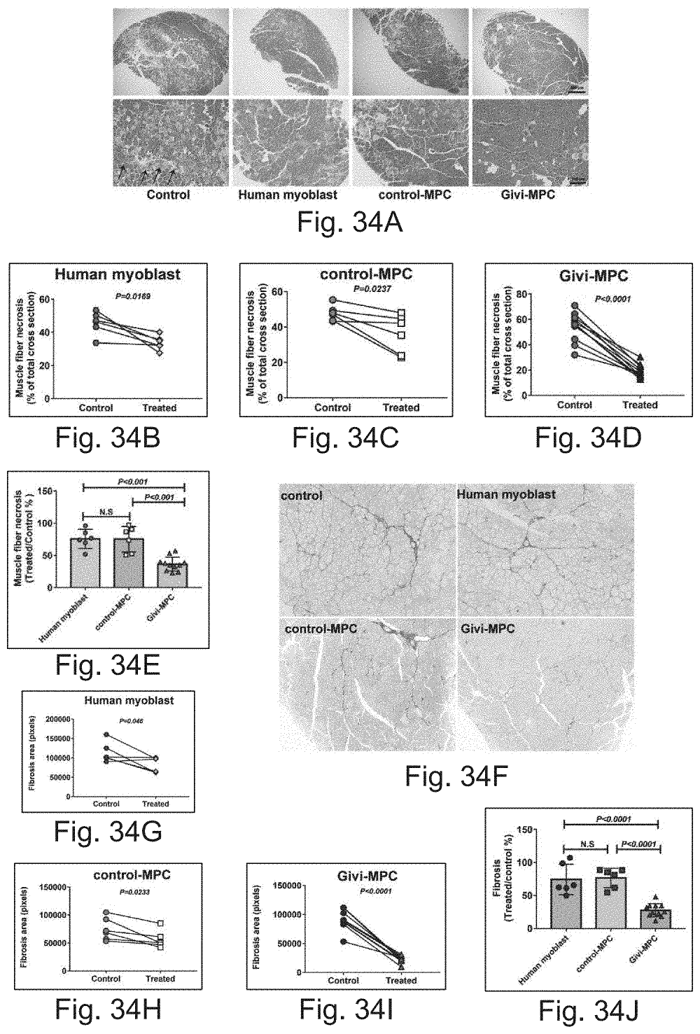

A significant number of Givi-MPC were transformed into muscle stem cells and occupied their sites as evidenced by double positivity for Pax7 and HNA cell under basal lamina at 1M post-transplantation (FIG. 35A). A schematic outline of reinjury experiments with CTX is provided (FIG. 35B). Compared with contralateral PBS treated TA muscle, expression of dystrophin was detected in Givi-MPC treated TA muscle after reinjury (FIG. 35C). Furthermore, Givi-MPC treated TA muscle showed increased muscle regeneration and less infiltration of inflammatory cells compared with contralateral PBS treated muscle (FIG. 35D). These data indicated that the engrafted Pax7 positive cells responded to reinjury and formed new muscle fibers.

Extracellular Vesicles Derived from Givi-MPC Facilitated Angiogenesis in Muscle Following CTX Injury

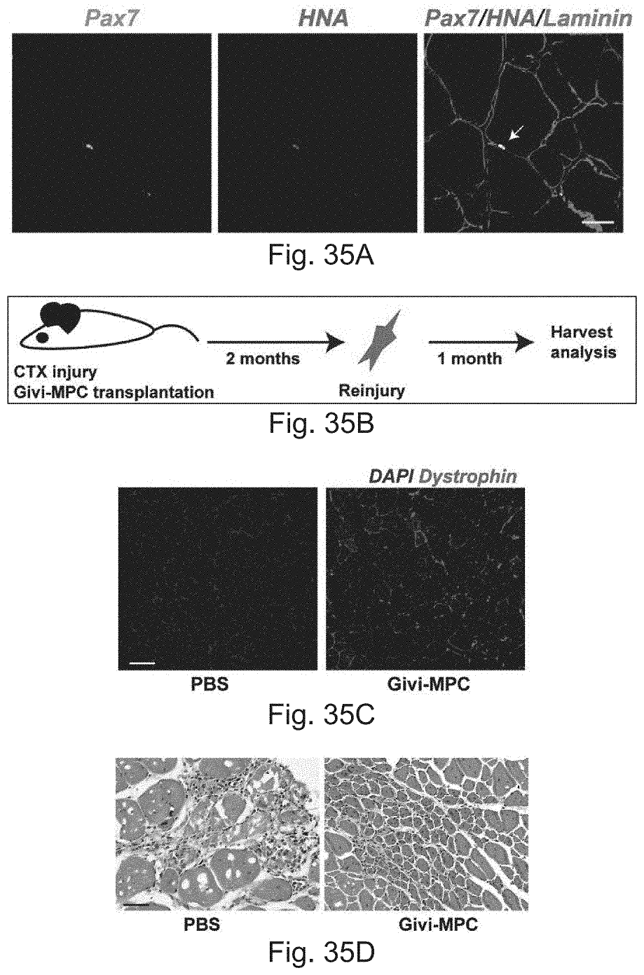

Angiogenesis is critical for muscle regeneration (18,19). Givi-MPC treatment caused higher capillary density (CD31 positivity) in TA muscle 1M post CTX injury (FIGS. 36A and 36B). Next we tested whether increased angiogenesis was due to paracrine effects by EV released from MPCs. We isolated EV from Givi-MPC using size exclusion columns. Using tunable resistive pulse sensing (TRPS) technique, we measured the concentration and size of EV from Givi-MPC. The size of isolated EV was roughly 118 31.7 nm. In vitro tube formation assay indicated that EV from Givi-MPC promoted tube formation (FIG. 36C) and resulted in higher average tube length (FIG. 36D) compared to treatment with EV from human myoblasts or control-MPC. We further analyzed the miRNA cargo contents of EV from Givi-MPC. A heatmap of significantly upregulated and downregulated miRNAs in EV from Givi-MPC compared to EV from human myoblasts was shown in FIG. 36E. miR-210, miR-181a, miR-17 and miR-107 were enriched in EV from Givi-MPC.

Discussion

In the present study, we successfully generated highly proliferative and integration free MPC from multiple hiPS cell lines using CHIR99021 and Givi. These cells expressed myogenic markers including Pax7 and desmin, which were also capable to differentiate into muscle cells under specific differentiation medium in vitro. Of particular significance was the ability of these MPCs to differentiate in dystrophic mouse model, making them more suitable for therapeutic applications. These cells possess special properties which make them unique for therapeutic applications. Migration and engraftment of transplanted cells to the site of injury are crucial to initiate differentiation into skeletal muscle components in the dystrophic muscle(20,21). Limited cell migration hampers engraftment efficiency in skeletal muscle (22,23). In the present study, we found MPC induced by Givi exhibited superior migration and proliferation capabilities compared with human myoblasts and control MPC generated by CHIR99021 and FGF. Go analysis further showed upregulation of cell migration related genes enabling them to migrate to distant injured muscle (10). In our data, genes related to migration were significantly upregulated with Givi treatment. ITGA4 was the most upregulated gene with 25.61-fold change. Integrin subunit α4 (ITGA4) is a member of the integrin alpha chain family of proteins. Integrin a subunits which pair with 1 play a critical role during in vivo myogenesis. Integrin α4 subunit is expressed in the myotome and in early limb muscle masses during muscle development (24,25). Murine Lbax1⁺ embryonic muscle progenitors expressed ITGA4 (26). It has been reported that teratoma derived MPC possessed high engraftment efficiency in muscle dystrophy model (10). However, the mechanism of upregulation of ITGA4 by Givi and migration medicated by ITGA4 need further study. DMD is a disease with body-wide systemic and progressive skeletal muscle loss, thus further study for the role and mechanism of ITGA4 in MPC migration will move MPC-based therapy for DMD forward to clinical application. In agreement with in vitro observations, we also observed higher engraftment efficiency of Givi-MPC compared to human myoblasts and control MPC upon transplantation in muscle tissue from Mdx/SCID mice following CTX injury. The significant engraftment in muscles of Mdx/SCID mice by human iPS-derived skeletal myogenic progenitors resulted in more dystrophin expressing myofibers or human laminin positive myofibers. Besides dystrophin, presence of neuromuscular junctions in human myofibers using α-BTX together with dystrophin in Mdx/SCID mice with Givi-MPC transplantation, suggest that formation of functional myofibers has occurred.

Histological analysis showed that fewer muscle fibers had undergone necrosis and fibrosis in injured TA muscle of Mdx/SCID mice treated with Givi-MPC. Inflammatory cell infiltration in general contributes to myofiber necrosis (27,28). Although Mdx/SCID mice are immunodeficient, it has been reported that M1 macrophages participated in skeletal muscle regeneration in SCID mice (29), suggesting partial immune reactivity in these mice. It has been reported Givi has potential anti-inflammatory effects (30,31). For example, Givi decreased inflammation in a mouse model with myocardial infarction (31). With HE staining, we found infiltration of larger number of inflammatory cells in TA muscle from Mdx/SCID mice treated with PBS, or human myoblasts or control MPC treatments 7 days post-CTX injury. Negligible macrophage infiltration identified by CD68 staining was observed in Givi-MPC transplanted Mdx/SCID mice 7 day post-CTX injury. These observations support that Givi-MPC had anti-inflammatory effects upon transplantation in CTX injured muscle suggesting that properties of MPC depend on the source of reprogramming molecule. Besides immediate effects on engraftment and differentiation, the long-term maintenance of newly formed skeletal muscle is ultimately dependent on the ability of the transplanted MPCs to contribute to the skeletal muscle stem cell pool (10). Here we observed Givi-MPC derived Pax7 positive cells under basal lamina upon transplantation, and with subsequent reinjury the Givi-MPC contributed to secondary regeneration in the Mdx/SCID mice. This observation supported that a subpopulation of Givi-MPC can seed the stem cell pool.

Angiogenic impairment of the vascular endothelial cells (EC) isolated from mdx mice compared with wild type mice has been reported (32) causing a marked decrease in the vasculature in TA muscle of mdx mice (33). Local delivery of muscle-derived stem cells engineered to overexpress human VEGF into the gastrocnemius muscle of Mdx/SCID mice resulted in marked increase in angiogenesis accompanied by enhanced muscle regeneration and decreased fibrosis compared with mice treated with non-engineered cells (34). In addition, satellite cells isolated from mdx mice exhibited reduced capacity to promote angiogenesis, as demonstrated in a co-culture model of satellite cells of Mdx mice and microvascular fragments (35). Here, our study demonstrated that after Givi-MPC transplantation, an increase in capillary density was observed as evidenced by CD31 staining in CTX injured Mdx/SCID mice compared to treatment with other MPCs. These results enforce the idea that an interaction between EC and MPC was important for myogenesis and angiogenesis in vitro and in vivo during skeletal muscle regeneration (18). To further strengthen this observation, we found that EV from Givi-iMPC were enriched in several miRNAs including miR-181a, miR-17, miR-210 and miR-107, miR-19b compared with EV from human myoblasts. Due to role of EV in cell-to-cell communication, these enriched miRNAs have been demonstrated to participate in angiogenesis. Activation of miR-17-92 cluster promoted angiogenesis via PTEN signaling pathway, however, EC miR-17-92 cluster knockout impaired angiogenesis (36). miR-181a and miR-210 are also reported to promote angiogenesis (37-40). Thus it is very likely that Givi-MPC interacted with resident EC to initiate myogenesis and angiogenesis in Mdx/SCID mice after CTX injury.

### Conclusion

We successfully generated highly expandable and integration free MPC from multiple hiPS cell lines using CHIR99021 and givinostat. Givinostat-induced MPC were highly proliferative and migratory and transplantation resulted in marked and impressive myoangiogenesis and restored dystrophin in injured TA muscle compared to control MPC and adult human myoblasts. It is concluded that hiPSCs pharmacologically reprogrammed into MPC with a small molecule, givinostat with anti-oxidative, anti-inflammatory and muscle gene promoting properties is an effective cellular source for treatment of muscle injury and restoration of dystrophin in DMD.

Funding

This study was supported by National Institutes of Health grants RO1 HL134354 & RO1 AR070029 (M Ashraf, Y Tang, and NL Weintraub).

Figure Legend

FIG. 29 Generation of muscle progenitor cell (MPC) from human iPSC using small molecules. (A) Schematic outline of generation of MPC from human PSC using combination of CHIR99021 and givinostat or CHIR99021 only. (B) Morphology of differentiating cells from 4 human iPSC lines (CF-iPSC, DF-iPSC-1, DF-iPSC-2 and DMD-iPSC) at 7 days. Bar=200 μm. (C) Morphology of replated MPC and differentiated myotubes from 4 human iPSC lines at day 14. Bar=200 μm

FIG. 30 Characterization of givinostat-induced MPC. (A) The treated hiPSC at day 14 expressed Pax7 and desmin. (B) The differentiated myotubes expressed MF20 as shown by immunostaining. Bar=50 μm.

FIG. 31 Givi-MPC exhibit superior proliferation and migration capacity. (A) Representative images and quantitative estimate (b) of cell migration by adult human myoblasts, and control-MPC, Givi-MPC (arrow). Cells were stained with Calcein AM (green). Bar=1 mm. (A) Quantitative estimate of migrated cells. Givi MPC showed highest number of cells migrated compared with human myoblasts (P<0.0001) or CHIR99021 induced MPC (P<0.0001). No significant difference was observed between human myoblasts and control-MPC. (C) Heat map of the Human RT² motility PCR Array. (D) Upregulated migration related genes in Givi-MPC vs. control-MPC using human cell motility PCR array. (E) The proliferation curves of human myoblasts vs MPC using CCK-8 assay. *P<0.05; ^{#}P<0.05 vs control-MPC. n=6. (F) Morphology of MPC colony. Bar=500 μm. Number of colonies (G) and percentage of colonies with different cell number (H). control-MPC: CHIR99021 induced MPC; Givi-MPC: CHIR99021 and Givinostat induced MPC.

FIG. 32 In vivo myogenic potential of different MPC and myoblast in Mdx/SCID mice with CTX injury. (A) Dystrophin restoration in Mdx/SCID mice by MPC transplantation at 1M after CTX injury. Bar=50 μm. (B). Transplanted cells were labeled with GFP (Green) and identified with human laminin staining (Red). Quantitation of engrafted fibers at 1M: Dystrophin+fibers (n=6) (C) and human laminin and GFP double positive fibers (n=3) (D). (E) Cross-section showing pre-synaptic staining with α-bungarotoxin in dystrophin positive fibers (n=3). Bar=20 μm.

FIG. 33 In vivo myogenic potential of different MPC and myoblast in Mdx/SCID mice with CTX injury. (A) Representative images of HE and Trichrome Masson staining in Mdx/SCID mice with human myoblasts or control-MPC or Givi-MPC transplantation 7 days after CTX injury. Black arrows indicate infiltrated inflammatory cells. (B) Quantification of muscle fiber necrosis between PBS treated collateral TA muscle or Givi-MPC treated TA muscle 7 days after CTX injury. (C) Quantification of muscle fiber necrosis of TA muscle among human myoblast or control-MPC or Givi-MPC transplantation mice 7 days after CTX injury. (D) Quantification of CD68 positive cells in TA muscle following MPC transplantation 7 days after CTX injury. (E) Representative images of macrophages (red, CD68) and human cells in TA muscle of Mdx/SCID mice with CTX injury following MPC transplantation.

FIG. 34 Givi-MPC decrease muscle necrosis and fibrosis in Mdx/SCID mice 1M after CTX injury. (A) Representative images of HE and Trichrome Masson staining in Mdx/SCID mice after transplantation with human myoblasts or control-MPC or Givi-MPC 1M after CTX injury. Bar=500 μm (4×) and Bar=100 μm (20×). Quantification of necrotic muscle fibers after treatment with human myoblasts (B), control-MPC (C) and Givi-MPC (D) 1M after CTX injury. (E) Comparison of muscle necrosis among human myoblasts or control-MPC or Givi-MPC transplantation mdx/SCID mice. (F) Representative images of tissue stained with Sirius red from Mdx/SCID mice. Bar=100 μm. Quantification of muscle fiber fibrosis in collateral TA muscle treated with human myoblasts (G), control-MPC (H) and Givi-MPC (I) 1M after CTX injury. (J) Muscle fibrosis after MPC transplantation in mdx/SCID mice.

FIG. 35 Givi-MPC repopulated the muscle stem cell pool. (A) Muscle cells positive for Pax7 (green) and human nuclear antigen (red) cell under the basal lamina from Mdx/SCID mice after 1M of Givi-MPC transplantation. Bar=20 μm. (B) Schematic of reinjury experiment. (C) 1M after reinjury, expression of dystrophin in Givi-MPC treated TA muscle tissue and contralateral PBS treated TA muscle tissue. Bar=50 μm. (D) Representative HE stained images of Givi-MPC treated TA muscle tissue and contralateral PBS treated TA muscle tissue. Bar=50 μm.

FIG. 36 Extracellular vesicles derived from Givi-MPC promoted angiogenesis. (A) Representative images of CD31 (Red) and laminin (Green) staining in Mdx/SCID mice 1M post injury. Bar=50 μm. (B) Quantification of capillary density (CD31 positive capillaries). (C) Representative images of tube formation by human aortic endothelia cells (HAECs) following EV treatment from human myoblasts, or control-MPC or Givi-MPC (1 μg/well, 24 well plate). HAECs were labeled with Calcein AM (Green). Bar=500 μm. (D) Tube formation assay. Average tube length was analyzed from 3 biological repeated experiments. (E) Heatmap showing significant upregulation of miRs in EV derived from Givi-MPC compared to EV-human myoblasts.

### REFERENCE

1. Yin H, Price F, Rudnicki M A. Satellite cells and the muscle stem cell niche. Physiol Rev 2013; 93:23-67.
2. Dayanidhi S, Lieber R L. Skeletal muscle satellite cells: mediators of muscle growth during development and implications for developmental disorders. Muscle Nerve 2014; 50:723-32.
3. Xu X, Wilschut K J, Kouklis G et al. Human Satellite Cell Transplantation and Regeneration from Diverse Skeletal Muscles. Stem Cell Reports 2015; 5:419-34.
4. Montarras D, Morgan J, Collins C et al. Direct isolation of satellite cells for skeletal muscle regeneration. Science 2005; 309:2064-7.
5. Sacco A, Doyonnas R, Kraft P, Vitorovic S, Blau H M. Self-renewal and expansion of single transplanted muscle stem cells. Nature 2008; 456:502-6.
6. Brimah K, Ehrhardt J, Mouly V, Butler-Browne G S, Partridge T A, Morgan J E. Human muscle precursor cell regeneration in the mouse host is enhanced by growth factors. Hum Gene Ther 2004; 15:1109-24.
7. Shelton M, Metz J, Liu J et al. Derivation and expansion of PAX7-positive muscle progenitors from human and mouse embryonic stem cells. Stem Cell Reports 2014; 3:516-29.
8. Chal J, Oginuma M, Al Tanoury Z et al. Differentiation of pluripotent stem cells to muscle fiber to model Duchenne muscular dystrophy. Nat Biotechnol 2015; 33:962-9.
9. Hicks M R, Hiserodt J, Paras K et al. ERBB3 and NGFR mark a distinct skeletal muscle progenitor cell in human development and hPSCs. Nat Cell Biol 2018; 20:46-57.
10. Chan S S, Arpke R W, Filareto A et al. Skeletal Muscle Stem Cells from PSC-Derived Teratomas Have Functional Regenerative Capacity. Cell Stem Cell 2018; 23:74-85 e6.
11. Consalvi S, Mozzetta C, Bettica P et al. Preclinical studies in the mdx mouse model of duchenne muscular dystrophy with the histone deacetylase inhibitor givinostat. Mol Med 2013; 19:79-87.
12. Saccone V, Consalvi S, Giordani L et al. HDAC-regulated myomiRs control BAF60 variant exchange and direct the functional phenotype of fibro-adipogenic progenitors in dystrophic muscles. Genes Dev 2014; 28:841-57.
13. Consalvi S, Sandona M, Saccone V. Epigenetic Reprogramming of Muscle Progenitors: Inspiration for Clinical Therapies. Stem Cells Int 2016; 2016:6093601.
14. Lewandowski J, Rozwadowska N, Kolanowski T J et al. The impact of in vitro cell culture duration on the maturation of human cardiomyocytes derived from induced pluripotent stem cells of myogenic origin. Cell Transplant 2018; 27:1047-1067.
15. Shavlakadze T, White J, Hoh J F, Rosenthal N, Grounds M D. Targeted expression of insulin-like growth factor-I reduces early myofiber necrosis in dystrophic mdx mice. Mol Ther 2004; 10:829-43.
16. Xuan W, Wang L, Xu M, Weintraub N L, Ashraf M. miRNAs in Extracellular Vesicles from iPS-Derived Cardiac Progenitor Cells Effectively Reduce Fibrosis and Promote Angiogenesis in Infarcted Heart. Stem Cells Int 2019; 2019:3726392.
17. Vogel R, Coumans F A, Maltesen R G et al. A standardized method to determine the concentration of extracellular vesicles using tunable resistive pulse sensing. J Extracell Vesicles 2016; 5:31242.
18. Latroche C, Weiss-Gayet M, Muller L et al. Coupling between Myogenesis and Angiogenesis during Skeletal Muscle Regeneration Is Stimulated by Restorative Macrophages. Stem Cell Reports 2017; 9:2018-2033.
19. Hardy D, Fefeu M, Besnard A et al. Defective angiogenesis in CXCL12 mutant mice impairs skeletal muscle regeneration. Skelet Muscle 2019; 9:25.
20. Xiao L, Lee K K. BRE facilitates skeletal muscle regeneration by promoting satellite cell motility and differentiation. Biol Open 2016; 5:100-11.
21. Otto A, Collins-Hooper H, Patel A, Dash P R, Patel K. Adult skeletal muscle stem cell migration is mediated by a blebbing/amoeboid mechanism. Rejuvenation Res 2011; 14:249-60.
22. Maffioletti S M, Noviello M, English K, Tedesco F S. Stem cell transplantation for muscular dystrophy: the challenge of immune response. Biomed Res Int 2014; 2014:964010.
23. Sun C, Serra C, Lee G, Wagner K R. Stem cell-based therapies for Duchenne muscular dystrophy. Exp Neurol 2020; 323:113086.
24. Bajanca F, Thorsteinsdottir S. Integrin expression patterns during early limb muscle development in the mouse. Gene Expr Patterns 2002; 2:133-6.
25. Cachaco A S, Pereira C S, Pardal R G, Bajanca F, Thorsteinsdottir S. Integrin repertoire on myogenic cells changes during the course of primary myogenesis in the mouse. Dev Dyn 2005; 232:1069-78.
26. Shelton M, Ritso M, Liu J et al. Gene expression profiling of skeletal myogenesis in human embryonic stem cells reveals a potential cascade of transcription factors regulating stages of myogenesis, including quiescent/activated satellite cell-like gene expression. PLoS One 2019; 14:e0222946.
27. Klyen B R, Shavlakadze T, Radley-Crabb H G, Grounds M D, Sampson D D. Identification of muscle necrosis in the mdx mouse model of Duchenne muscular dystrophy using three-dimensional optical coherence tomography. J Biomed Opt 2011; 16:076013.
28. Roche J A, Tulapurkar M E, Mueller A L et al. Myofiber damage precedes macrophage infiltration after in vivo injury in dysferlin-deficient A/J mouse skeletal muscle. Am J Pathol 2015; 185:1686-98.
29. Grabowska I, Mazur M A, Kowalski K et al. Progression of inflammation during immunodeficient mouse skeletal muscle regeneration. J Muscle Res Cell Motil 2015; 36:395-404.
30. Furlan A, Monzani V, Reznikov L L et al. Pharmacokinetics, safety and inducible cytokine responses during a phase 1 trial of the oral histone deacetylase inhibitor ITF2357 (givinostat). Mol Med 2011; 17:353-62.
31. Milan M, Pace V, Maiullari F et al. Givinostat reduces adverse cardiac remodeling through regulating fibroblasts activation. Cell Death Dis 2018; 9:108.
32. Palladino M, Gatto I, Neri V et al. Angiogenic impairment of the vascular endothelium: a novel mechanism and potential therapeutic target in muscular dystrophy. Arterioscler Thromb Vasc Biol 2013; 33:2867-76.
33. Matsakas A, Yadav V, Lorca S, Narkar V. Muscle ERRgamma mitigates Duchenne muscular dystrophy via metabolic and angiogenic reprogramming. FASEB J 2013; 27:4004-16.
34. Deasy B M, Feduska J M, Payne T R, Li Y, Ambrosio F, Huard J. Effect of VEGF on the regenerative capacity of muscle stem cells in dystrophic skeletal muscle. Mol Ther 2009; 17:1788-98.
35. Rhoads R P, Johnson R M, Rathbone C R et al. Satellite cell-mediated angiogenesis in vitro coincides with a functional hypoxia-inducible factor pathway. Am J Physiol Cell Physiol 2009; 296:C1321-8.
36. Chamorro-Jorganes A, Lee M Y, Araldi E et al. VEGF-Induced Expression of miR-17-92 Cluster in Endothelial Cells Is Mediated by ERK/ELK1 Activation and Regulates Angiogenesis. Circ Res 2016; 118:38-47.
37. Sun W, Wang X, Li J et al. MicroRNA-181a promotes angiogenesis in colorectal cancer by targeting SRCIN1 to promote the SRC/VEGF signaling pathway. Cell Death Dis 2018; 9:438.
38. Zaccagnini G, Maimone B, Fuschi P et al. Overexpression of miR-210 and its significance in ischemic tissue damage. Sci Rep 2017; 7:9563.
39. Fan Z G, Qu X L, Chu P et al. MicroRNA-210 promotes angiogenesis in acute myocardial infarction. Mol Med Rep 2018; 17:5658-5665.
40. Zhang H, Wu J, Wu J et al. Exosome-mediated targeted delivery of miR-210 for angiogenic therapy after cerebral ischemia in mice. J Nanobiotechnology 2019; 17:29.

Each of the following is incorporated by reference in its entirety for all purposes.
1. Xuan W, et al., Cardiac Progenitors Induced from Human Induced Pluripotent Stem Cells with Cardiogenic Small Molecule Effectively Regenerate Infarcted Hearts and Attenuate Fibrosis. Shock 2018, 50(6):627-639.
2. Chen G, et al., Marrow stromal cells for cell-based therapy: the role of antiinflammatory cytokines in cellular cardiomyoplasty. Ann Thorac Surg 2010, 90(1):190-197.
3. Lai R C, Y et al., Exosomes for drug delivery—a novel application for the mesenchymal stem cell. Biotechnol Adv 2013, 31(5):543-551.
4. Hu G W, et al., Exosomes secreted by human-induced pluripotent stem cell-derived mesenchymal stem cells attenuate limb ischemia by promoting angiogenesis in mice. Stem Cell Res Ther 2015, 6:10.
5. Yuana Y, et al., Cryo-electron microscopy of extracellular vesicles in fresh plasma. J Extracell Vesicles 2013, 2.
6. Wang X, Gu H, Qin D, Yang L, Huang W, Essandoh K, Wang Y, Caldwell C C, Peng T, Zingarelli B et al: Exosomal miRNA-223 Contributes to Mesenchymal Stem Cell-Elicited Cardioprotection in Polymicrobial Sepsis. Sci Rep 2015, 5:13721.
7. Waldenstrom A, et al., Cardiomyocyte microvesicles contain DNA/RNA and convey biological messages to target cells. PLoS One 2012, 7(4):e34653.
8. Genneback N, et al., Growth factor stimulation of cardiomyocytes induces changes in the transcriptional contents of secreted exosomes. J Extracell Vesicles 2013, 2.
9. Khan M, et al., Embryonic stem cell-derived exosomes promote endogenous repair mechanisms and enhance cardiac function following myocardial infarction. Circ Res 2015, 117(1):52-64.
10. Ortiz L A, et al., Mesenchymal stem cell engraftment in lung is enhanced in response to bleomycin exposure and ameliorates its fibrotic effects. Proc Natl Acad Sci USA 2003, 100(14):8407-8411.
11. Weiss D J, et al., Stem cells and cell therapies in lung biology and lung diseases. Proc Am Thorac Soc 2011, 8(3):223-272.
12. Henning R J: Current status of stem cells in cardiac repair. Future Cardiol 2018, 14(2):181-192.
13. Ong S G, Wu J C: Exosomes as potential alternatives to stem cell therapy in mediating cardiac regeneration. Circ Res 2015, 117(1):7-9.
14. Ahmed R P, et al., Sonic Hedgehog gene delivery to the rodent heart promotes angiogenesis via iNOS/netrin-1/PKC pathway. PLoS One 2010, 5(1):e8576.
15. Lu G, et al., Sca-1+stem cell survival and engraftment in the infarcted heart: dual role for preconditioning-induced connexin-43. Circulation 2009, 119(19):2587-2596.
16. Zhao T, et al., Stem cell homing and angiomyogenesis in transplanted hearts are enhanced by combined intramyocardial SDF-1alpha delivery and endogenous cytokine signaling. Am J Physiol Heart Circ Physiol 2009, 296(4):H976-986.
17. Haider H, et al., IGF-1-overexpressing mesenchymal stem cells accelerate bone marrow stem cell mobilization via paracrine activation of SDF-1alpha/CXCR4 signaling to promote myocardial repair. Circ Res 2008, 103(11):1300-1308.
18. Haider H, et al., Angiomyogenesis for cardiac repair using human myoblasts as carriers of human vascular endothelial growth factor. J Mol Med (Berl) 2004, 82(8):539-549.
19. Uemura R, et al., Bone marrow stem cells prevent left ventricular remodeling of ischemic heart through paracrine signaling. Circ Res 2006, 98(11):1414-1421.
20. Wang Y, et al., Combining pharmacological mobilization with intramyocardial delivery of bone marrow cells over-expressing VEGF is more effective for cardiac repair. J Mol Cell Cardiol 2006, 40(5):736-745.
21. Niagara M I, et al., Pharmacologically preconditioned skeletal myoblasts are resistant to oxidative stress and promote angiomyogenesis via release of paracrine factors in the infarcted heart. Circ Res 2007, 100(4):545-555.
22. Jiang S, H et al., Supportive interaction between cell survival signaling and angiocompetent factors enhances donor cell survival and promotes angiomyogenesis for cardiac repair. Circ Res 2006, 99(7):776-784.
23. Burchfield J S, et al., Pharmacological priming of adipose-derived stem cells promotes myocardial repair. J Investig Med 2016, 64(1):50-62.
24. Kato M, et al., Cell-free formation of RNA granules: low complexity sequence domains form dynamic fibers within hydrogels. Cell 2012, 149(4):753-767.
25. Han T W, et al., Cell-free formation of RNA granules: bound RNAs identify features and components of cellular assemblies. Cell 2012, 149(4):768-779.
26. Chong J J, et al., Human embryonic-stem-cell-derived cardiomyocytes regenerate non-human primate hearts. Nature 2014, 510(7504):273-277.
27. Zhang Y, et al., Expandable Cardiovascular Progenitor Cells Reprogrammed from Fibroblasts. Cell Stem Cell 2016, 18(3):368-381.
28. Oh H, B et al., Cardiac progenitor cells from adult myocardium: homing, differentiation, and fusion after infarction. Proc Natl Acad Sci USA 2003, 100(21):12313-12318.
29. Shiba Y, et al., Human ES-cell-derived cardiomyocytes electrically couple and suppress arrhythmias in injured hearts. Nature 2012, 489(7415):322-325.
30. Blin G, et al., A purified population of multipotent cardiovascular progenitors derived from primate pluripotent stem cells engrafts in postmyocardial infarcted nonhuman primates. J Clin Invest 2010, 120(4):1125-1139.
31. Christoforou N, et al., Implantation of mouse embryonic stem cell-derived cardiac progenitor cells preserves function of infarcted murine hearts. PLoS One 2010, 5(7):e11536.
32. Ye L, et al., Cardiac repair in a porcine model of acute myocardial infarction with human induced pluripotent stem cell-derived cardiovascular cells. Cell Stem Cell 2014, 15(6):750-761.
33. Laugwitz K L, et al., Postnatal is 11+ cardioblasts enter fully differentiated cardiomyocyte lineages. Nature 2005, 433(7026):647-653.
34. Moretti A, et al., Multipotent embryonic is 11+ progenitor cells lead to cardiac, smooth muscle, and endothelial cell diversification. Cell 2006, 127(6):1151-1165.
35. Koninckx R, et al., The cardiac atrial appendage stem cell: a new and promising candidate for myocardial repair. Cardiovasc Res 2013, 97(3):413-423.
36. Lei I L, Bu L, Wang Z: Derivation of cardiac progenitor cells from embryonic stem cells. J Vis Exp 2015(95):52047.
37. Yang L, et al., Human cardiovascular progenitor cells develop from a KDR+embryonic-stem-cell-derived population. Nature 2008, 453(7194):524-528.
38. Xu Y, Shi Y, Ding S: A chemical approach to stem-cell biology and regenerative medicine. Nature 2008, 453(7193):338-344.
39. Graichen R, et al., Enhanced cardiomyogenesis of human embryonic stem cells by a small molecular inhibitor of p38 MAPK. Differentiation 2008, 76(4):357-370.
40. Hansson E M, Lindsay M E, Chien K R: Regeneration next: toward heart stem cell therapeutics. Cell Stem Cell 2009, 5(4):364-377.
41. Hao J, et al., Dorsomorphin, a selective small molecule inhibitor of BMP signaling, promotes cardiomyogenesis in embryonic stem cells. PLoS One 2008, 3(8):e2904.
42. Naito A T, et al., Developmental stage-specific biphasic roles of Wnt/beta-catenin signaling in cardiomyogenesis and hematopoiesis. Proc Natl Acad Sci USA 2006, 103(52):19812-19817.
43. Qyang Y, et al., The renewal and differentiation of Is 11+ cardiovascular progenitors are controlled by a Wnt/beta-catenin pathway. Cell Stem Cell 2007, 1(2):165-179.
44. Cao N, et al., Conversion of human fibroblasts into functional cardiomyocytes by small molecules. Science 2016, 352(6290):1216-1220.
45. Sadek H, et al., Cardiogenic small molecules that enhance myocardial repair by stem cells. Proc Natl Acad Sci USA 2008, 105(16):6063-6068.
46. Velasquez L S, et al., Small EM: Activation of MRTF-A-dependent gene expression with a small molecule promotes myofibroblast differentiation and wound healing. Proc Natl Acad Sci USA 2013, 110(42):16850-16855.
47. Koh S H, et al., Differential Effects of Isoxazole-9 on Neural Stem/Progenitor Cells, Oligodendrocyte Precursor Cells, and Endothelial Progenitor Cells. PLoS One 2015, 10(9):e0138724.
48. Drowley L, et al., Human Induced Pluripotent Stem Cell-Derived Cardiac Progenitor Cells in Phenotypic Screening: A Transforming Growth Factor-beta Type 1 Receptor Kinase Inhibitor Induces Efficient Cardiac Differentiation. Stem Cells Transl Med 2016, 5(2):164-174.
49. Thery C, Zitvogel L, Amigorena S: Exosomes: composition, biogenesis and function. Nat Rev Immunol 2002, 2(8):569-579.
50. Dorronsoro A, Robbins P D: Regenerating the injured kidney with human umbilical cord mesenchymal stem cell-derived exosomes. Stem Cell Res Ther 2013, 4(2):39.
51. Simons M, Raposo G: Exosomes—vesicular carriers for intercellular communication. Curr Opin Cell Biol 2009, 21(4):575-581.
52. Terrisse A D, et al., Internalization of microparticles by endothelial cells promotes platelet/endothelial cell interaction under flow. J Thromb Haemost 2010, 8(12):2810-2819.
53. Diehl P, et al., Microparticles: major transport vehicles for distinct microRNAs in circulation. Cardiovasc Res 2012, 93(4):633-644.
54. Burger D, et al., Microparticles: biomarkers and beyond. Clin Sci (Lond) 2013, 124(7):423-441.
55. Feng Y, et al., Ischemic preconditioning potentiates the protective effect of stem cells through secretion of exosomes by targeting Mecp2 via miRNA-22. PLoS One 2014, 9(2):e88685.
56. Zhang J, et al., Exosome and exosomal microRNA: trafficking, sorting, and function. Genomics Proteomics Bioinformatics 2015, 13(1):17-24.
57. Heidary S, et al., Quantitative tissue characterization of infarct core and border zone in patients with ischemic cardiomyopathy by magnetic resonance is associated with future cardiovascular events. J Am Coll Cardiol 2010, 55(24):2762-2768.
58. Gray W D, et al., Identification of therapeutic covariant microRNA clusters in hypoxia-treated cardiac progenitor cell exosomes using systems biology. Circ Res 2015, 116(2):255-263.
59. Timmers L, et al., Human mesenchymal stem cell-conditioned medium improves cardiac function following myocardial infarction. Stem Cell Res 2011, 6(3):206-214.
60. Lai R C, Chen T S, Lim S K: Mesenchymal stem cell exosome: a novel stem cell-based therapy for cardiovascular disease. Regen Med 2011, 6(4):481-492.
61. Lai R C, et al., Proteolytic Potential of the MSC Exosome Proteome: Implications for an Exosome-Mediated Delivery of Therapeutic Proteasome. Int J Proteomics 2012, 2012:971907.
62. Zhao Y, et al., Exosomes Derived from Human Umbilical Cord Mesenchymal Stem Cells Relieve Acute Myocardial Ischemic Injury. Stem Cells Int 2015, 2015:761643.
63. Yu B, et al., Exosomes secreted from GATA-4 overexpressing mesenchymal stem cells serve as a reservoir of anti-apoptotic microRNAs for cardioprotection. Int J Cardiol 2015, 182:349-360.
64. Zhang B, et al., Human umbilical cord mesenchymal stem cell exosomes enhance angiogenesis through the Wnt4/beta-catenin pathway. Stem Cells Transl Med 2015, 4(5):513-522.
65. Arslan F, et al., Mesenchymal stem cell-derived exosomes increase ATP levels, decrease oxidative stress and activate PI3K/Akt pathway to enhance myocardial viability and prevent adverse remodeling after myocardial ischemia/reperfusion injury. Stem Cell Res 2013, 10(3):301-312.
66. Vicencio J M, et al., Plasma exosomes protect the myocardium from ischemia-reperfusion injury. J Am Coll Cardiol 2015, 65(15):1525-1536.
67. Colombo M, Raposo G, Thery C: Biogenesis, secretion, and intercellular interactions of exosomes and other extracellular vesicles. Annu Rev Cell Dev Biol 2014, 30:255-289.
68. Lee C, et al., Exosomes mediate the cytoprotective action of mesenchymal stromal cells on hypoxia-induced pulmonary hypertension. Circulation 2012, 126(22):2601-2611.
69. Yang X, Cohen M V, Downey J M: Mechanism of cardioprotection by early ischemic preconditioning. Cardiovasc Drugs Ther 2010, 24(3):225-234.
70. Cohen M V, Downey J M: Is it time to translate ischemic preconditioning's mechanism of cardioprotection into clinical practice? J Cardiovasc Pharmacol Ther 2011, 16(3-4):273-280.
71. Haider H, Ashraf M: Strategies to promote donor cell survival: combining preconditioning approach with stem cell transplantation. J Mol Cell Cardiol 2008, 45(4):554-566.
72. Bonauer A, Boon R A, Dimmeler S: Vascular microRNAs. Curr Drug Targets 2010, 11(8):943-949.
73. Suarez Y, Sessa W C: MicroRNAs as novel regulators of angiogenesis. Circ Res 2009, 104(4):442-454.
74. Vincenti S, et al., HUVEC respond to radiation by inducing the expression of pro-angiogenic microRNAs. Radiat Res 2011, 175(5):535-546.
75. Kim H W, et al., Ischemic preconditioning augments survival of stem cells via miRNA-210 expression by targeting caspase-8-associated protein 2. J Biol Chem 2009, 284(48):33161-33168.
76. Leroux L, et al., Hypoxia preconditioned mesenchymal stem cells improve vascular and skeletal muscle fiber regeneration after ischemia through a Wnt4-dependent pathway. Mol Ther 2010, 18(8):1545-1552.
77. Valadi H, E et al., Exosome-mediated transfer of mRNAs and microRNAs is a novel mechanism of genetic exchange between cells. Nat Cell Biol 2007, 9(6):654-659.
78. Yuan A, et al., Transfer of microRNAs by embryonic stem cell microvesicles. PLoS One 2009, 4(3):e4722.
79. Suzuki Y, et al., Diazoxide potentiates mesenchymal stem cell survival via NF-kappaB-dependent miRNA-146a expression by targeting Fas. Am J Physiol Heart Circ Physiol 2010, 299(4):H1077-1082.
80. Caspi O, et al., Transplantation of human embryonic stem cell-derived cardiomyocytes improves myocardial performance in infarcted rat hearts. J Am Coll Cardiol 2007, 50(19):1884-1893.
81. Ong S G, et al., Microfluidic Single-Cell Analysis of Transplanted Human Induced Pluripotent Stem Cell-Derived Cardiomyocytes After Acute Myocardial Infarction. Circulation 2015, 132(8):762-771.
82. Lam J T, Moretti A, Laugwitz K L: Multipotent progenitor cells in regenerative cardiovascular medicine. Pediatr Cardiol 2009, 30(5):690-698.
83. Ahmed R P, et al., Reprogramming of skeletal myoblasts for induction of pluripotency for tumor-free cardiomyogenesis in the infarcted heart. Circ Res 2011, 109(1):60-70.
84. Buccini S, et al., Cardiac progenitors derived from reprogrammed mesenchymal stem cells contribute to angiomyogenic repair of the infarcted heart. Basic Res Cardiol 2012, 107(6):301.
85. Pasha Z, Haider H, Ashraf M: Efficient non-viral reprogramming of myoblasts to stemness with a single small molecule to generate cardiac progenitor cells. PLoS One 2011, 6(8):e23667.
86. Biswas S K, Mantovani A: Macrophage plasticity and interaction with lymphocyte subsets: cancer as a paradigm. Nat Immunol 2010, 11(10):889-896.
87. Yang D, et al., EphrinB2/EphB4 pathway in postnatal angiogenesis: a potential therapeutic target for ischemic cardiovascular disease. Angiogenesis 2016, 19(3):297-309.
88. Gai H, et al., Generation and characterization of functional cardiomyocytes using induced pluripotent stem cells derived from human fibroblasts. Cell Biol Int 2009, 33(11):1184-1193.
89. Kuzmenkin A, et al., Functional characterization of cardiomyocytes derived from murine induced pluripotent stem cells in vitro. FASEB J 2009, 23(12):4168-4180.
90. Martinez-Fernandez A, et al., c-MYC independent nuclear reprogramming favors cardiogenic potential of induced pluripotent stem cells. J Cardiovasc Transl Res 2010, 3(1):13-23.
91. Nelson T J, et al., Repair of acute myocardial infarction by human stemness factors induced pluripotent stem cells. Circulation 2009, 120(5):408-416.
92. Pearl J I, et al., Short-term immunosuppression promotes engraftment of embryonic and induced pluripotent stem cells. Cell Stem Cell 2011, 8(3):309-317.
93. Salomon C, et al., Exosomal signaling during hypoxia mediates microvascular endothelial cell migration and vasculogenesis. PLoS One 2013, 8(7):e68451.
94. Bartosh T J, et al., Aggregation of human mesenchymal stromal cells (MSCs) into 3D spheroids enhances their antiinflammatory properties. Proc Natl Acad Sci USA 2010, 107(31):13724-13729.
95. Konoplyannikov M, et al., Activation of diverse signaling pathways by ex-vivo delivery of multiple cytokines for myocardial repair. Stem Cells Dev 2013, 22(2):204-215.
96. Okada M, et al., Abrogation of Age-Induced MicroRNA-195 Rejuvenates the Senescent Mesenchymal Stem Cells by Reactivating Telomerase. Stem Cells 2016, 34(1):148-159.
97. Pin Li, et al., Progress in Exosome Isolation Techniques, Theranostics. 2017; 7(3): 789-804.
98. Xuan W, et all., miRNAs in Extracellular Vesicles from iPS-Derived Cardiac Progenitor Cells Effectively Reduce Fibrosis and Promote Angiogenesis in Infarcted Heart. Stem Cells International Volume 2019 (2019), Article ID 3726392,
99. Xuan W, et all Human iPS Cells Derived Skeletal Muscle Progenitor Cells Promote Myoangiogenesis and Restore Dystrophin in Duchenne Muscular Dystrophic Mice. bioRxiv 2020.01.21.914283

1) A method of treating cardiac disease, comprising
i) administering a composition to a human patient having cardiac disease in an amount sufficient to treat said cardiac disease;
ii) said composition comprising:
i. allogenic or autologous cardiac progenitor cells (CPCs);
ii. plus additional extracellular vesicles derived from said CPCs or another population of CPCs.

2) The method of claim 1, wherein said allogenic or autologous CPCs are made by a process comprising:
i) isolating parent cells from said patient or a person allogenic to said patient, wherein said parent cells are either induced pluripotent stem cells (iPSCs) or pluripotent stem cells (PSCs);
ii) treating said parent cells in vitro with ISX-9 or Danazol or other isoxazole based compound or Givinostat or the combination of Givinostat and small molecule: CHIR99021, in an amount effective to induce differentiation of said iPSCs or PSCs into CPCs and/or smooth muscle cells, myocytes, endothelial cells, or muscle progenitor cells.
iii) Treating said parent cells in vitro with an isoxazole compound to induce differentiation of said iPSCs or PSCs into CPCs and/or smooth muscle cells, myocytes, endothelial cells, or muscle progenitor cells with an isoxazole formula of: 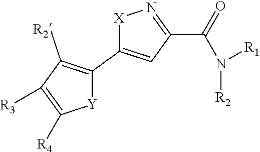
wherein R₁ and R₂ are both hydrogen or R₁ is hydrogen and R₂ is selected from the group consisting of substituted or unsubstituted C₁-C₆ alkyl, C₃-C₆ cycloalkyl, C₂-C₆ alkenyl, C₂-C₆ alkynyl, and benzyl, or where R₁ and R₂ may be joined together to form a ring selected from azetidinyl, pyrrolidinyl, piperidinyl, piperazinyl, or morpholinyl; R_{2′}, R₃ and R₄ are independently selected from the group consisting of hydrogen, halogen, C₁-C₆ alkyl, C₃-C₆ cycloalkyl, substituted or unsubstituted aromatic or heteroaromatic ring, cyano, nitro and acyl; X is O, NH or S; and Y is O, NH or S.

3) The method of claim 1, wherein said allogenic or autologous CPCs are made by a process comprising:
i) isolating parent cells from said patient or a person allogenic to said patient, wherein said parent cells are induced pluripotent stem cells (iPSCs) or pluripotent stem cells (PSCs) or multipotent stem cells (MSCs);
ii) culturing said parent cells with 0.1-35 μM ISX-9 or other isoxazole based compound for 3-10 days in a medium without insulin to induce parent cells to form CPCs;
iii) culturing said CPCs in a medium without ISX-9 or other isoxazole based compound and with insulin for 3-10 days to induce differentiation of said CPC cells into a mixture comprising CPCs and one or more of cardiomyocytes, smooth muscle cells and endothelial cells.

4) A method of treating cardiac disease, comprising:
i) administering a composition to a heart in a human patient having cardiac disease in an amount sufficient to treat said cardiac disease;
ii) said composition comprising:
i. allogenic or autologous cardiac progenitor cells (CPCs);
ii. plus added extracellular vesicles derived from said CPCs;
iii) said cells made by:
i. isolating parent cells from said patient or a person allogenic to said patient, wherein said parent cells are induced pluripotent stem cells (iPSCs) or pluripotent stem cells (PSCs) or multipotent stem cells (MSCs);
ii. culturing said parent cells with 0.1-35 μM ISX-9 or isoxazole based compound for 3-10 days in a medium without insulin to induce said parent cells to form CPCs;
iii. culturing said CPCs in a medium without ISX-9 or isoxazole based compound and with insulin for 3-10 days to induce differentiation of said CPC cells into a mixture comprising CPCs and one or more of cardiomyocytes, smooth muscle cells and endothelial cells.

5) The method of claim 1, wherein said CPCs are subjected to hypoxic preconditioning before use in said human patient.

6) The method of claim 1, wherein said extracellular vesicles comprise miRNA-373 or an miRNA-373 mimic or an expressible nucleotide sequence encoding miRNA-373 or an miRNA-373 mimic.

7) The method of claim 1, wherein said extracellular CPCs and/or vesicles comprise miRNA-373 or a mimic of miRNA-373 and/or a ephrinB2 protein.

8) The method of claim 1, wherein said extracellular vesicles are isolated from a culture of CPCs by ultracentrifugation, ultrafiltration, precipitation, immunoaffinity capture or combinations thereof.

9) The method of claim 1, wherein; 1×10⁸-9×10⁸ CPCs (100-900 million) and 10⁹-10¹² (one billion-one trillion) extracellular vesicles are administered by intramyocardial injection, catheter injection or direct injection.

10) A composition for treating fibrosis or cardiac disease, said composition comprising allogenic or autologous cardiac progenitor cells (CPCs) plus added extracellular vesicles derived from said CPCs in a pharmaceutically acceptable carrier.

11) The composition of claim 10, said CPCs and said extracellular vesicles in a ratio of about 10×-1000× extracellular vesicles to CPC's.

12) The composition of claim 10, said CPCs and/or said extracellular vesicles comprising an miRNA-373 or a mimic of miRNA-373 or an expressible nucleic acid encoding said miRNA-373 or said mimic of miRNA-373.

13) The composition of claim 10, further comprising an ephrinB2 protein.

14) A composition, comprising CPCs made by induction of stem cells with ISX-9, Danazol or other isoxazole based compound plus exosomes containing miRNA-373 or a mimic of miRNA-373 in a pharmaceutically acceptable carrier.

15) A method of treating fibrosis or cardiac disease, said method comprising treating a patient having fibrosis in an amount sufficient to reduce the gene expression or protein activity of growth differentiation factor 11 (GDF-11) and/or Rho-associated coiled-coil containing kinase-2 (ROCK-2).

16) A method of preparing a population of skeletal myogenic progenitors from a population of human induced pluripotent stem cells (hiPSCs) or other pluripotent stem cells, comprising contacting the hiPSCs or other pluripotent stem cells with an effective amount of Givinostat (GIV) or the combination of Givinostat and small. molecule: CHIR99021, optionally cultured in serum free media and for treating Duchenne Muscular Dystrophy (DMD), Becker Muscular Dystrophy, muscular dystrophy, sarcopenia, and other muscular and muscle loss diseases comprising administering to a subject in need thereof an effective amount of said population of cells and/or an effective amount of said population of cells and/or exosomes or microvesicles.

17) A method of reducing fibrosis, comprising treating a patient having fibrosis or cardiac disease with a pharmaceutically effective amount of an a MIR-373 mimic mimetic compound, oligonucleotide, recombinant AAV vector or viral vector, gene editing constructs such as CRISPR and gene therapy vectors or recombinant viral particle or other pharmaceutically acceptable carrier containing MIR-373.

18) The composition of claim 17, comprising of:
1:) A 22-26 base nucleotide strand with greater than 85% homology to the group of related miRNAs including miR-371a-5p, miR-371a-3p, miR-371b-5p, miR-371-3p, miR-372-5p, miR-372-3p, miR-373-3p, miR-373-5p, or their variants, which also contains a 6-base seed sequence identical to the conserved seed sequence found within this same group of miRNAs.
2:) A second 22-26 base nucleotide strand is significantly complementary to the first strand and has least one modified nucleotide(s), such that when the two strands bind one another the first strand has a 3′ nucleotide overhang relative to the second strand

19) The method of claim 2 further comprising the steps of:
iv) Treating said parent cells in vitro with an isoxazole compound with iPSCs or PSCs into CPCs and/or smooth muscle cells, myocytes, endothelial cells, or muscle progenitor cells with an isoxazole formula of: wherein the isoxazole compound has the formula: 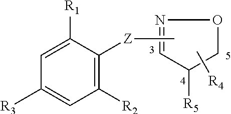
wherein R1 and R2 is each selected from C1-C4 alkyl, phenyl, benzyl, trifluoromethyl or halogen, R3 is selected from hydrogen, hydroxy, C1-C4 alkyl or alkoxy, R4, in position 3 or 5, is selected from hydrogen, trifluoromethyl, C1-C4 alkoxy, C1-C4 alkyl, or C1-C4 hydroxyalkyl, Rs is selected from hydrogen or C4-C4 alkyl or R4 and Rs together form a tetramethylene group, Z at position 3 or 5 on the heterocycle is selected from: —N(R6)-CO—, —CO—N(R6)-, —N(R6)-CO—N(R6)-, —CH(R6)-NH—CO—, or —NH—CO—CH(R6), in which R6 is selected from hydrogen or C1-C4 alkyl.
v.) Treating said parent cells in vitro with an isoxazole compound with iPSCs or PSCs into CPCs and/or smooth muscle cells, myocytes, endothelial cells, or muscle progenitor cells with an isoxazole formula of:
wherein the isoxazole compound is monosubstituted at the 3, 4, or 5 position with a substitutent selected from the group: hydrogen, alkyl, aryl, alkenyl, alkynyl, heterocylic, heteroaryl, carbonyl, carboxy, halogen, amine, sulfur, oxy, hydroxyl, mercapto, sulfinyl, sulfonyl, sulfide, thioamide, nitrile, nitro, stannyl, boronic acid, carboxylic acid, carboxylic acid derivative, alkoxyphenyl, haloalkyl, haloaryl, alkylaryl, nitroaryl, and morpholinoalkyl.
vi) Treating said parent cells in vitro with an isoxazole compound with iPSCs or PSCs into CPCs and/or smooth muscle cells, myocytes, endothelial cells, or muscle progenitor cells with an isoxazole formula of: wherein the isoxazole compound has the formula:
wherein the isoxazole is 3,5-disubstituted, 3,4,5-tri-substituted, or 4,5-disubstituted and the substituents are selected from the group: hydrogen, alkyl, aryl, alkenyl, alkynyl, heterocylic, heteroaryl, carbonyl, carboxy, halogen, amine, sulfur, oxy, hydroxyl, mercapto, sulfinyl, sulfonyl, sulfide, thioamide, nitrile, nitro, stannyl, boronic acid, carboxylic acid, carboxylic acid derivative, alkoxyphenyl, haloalkyl, haloaryl, alkylaryl, nitroaryl, and morpholinoalkyl.
